(19) United States
(12) Patent Application Publication
(10) Pub. No.: US 2025/0376508 A1
(43) Pub. Date: Dec. 11, 2025

(54) MONOCLONAL ANTIBODIES DIRECTED TO PHOSPHORYLATED PSD95 AND USES THEREOF

(71) Applicant: The United States of America, as represented by the Secretary, Dept. of Health and Human, Bethesda, MD (US)

(72) Inventors: Christopher Edward Ramsden, Bethesda, MD (US); Roy Gail Cutler, Reisterstown, MD (US)

(21) Appl. No.: 18/874,359

(22) PCT Filed: Jun. 30, 2023
(86) PCT No.: PCT/US2023/069503

§ 371 Date: Dec. 12, 2024

Related U.S. Application Data
(60) Provisional application No. 63/358,178, filed on Jul. 4, 2022.
(60) Provisional application No. 63/426,275, filed on Nov. 17, 2022.

(51) Int. Cl. C07K 16/18; A61K 49/00; G01N 33/68

(52) U.S. Cl. CPC C07K 16/18; A61K 49/0004; G01N 33/6896; C07K 2317/92; G01N 2800/2821

(57) ABSTRACT

The disclosure generally relates to immunoglobulin and/or antigen binding fragment(s) that specifically bind to post-synaptic density (PSD95) phosphorylated at threonine (19), at (serine 25), and/or at both threonine (19) and serine (25), as well as corresponding expression vectors and host cells, and methods of diagnosing and kit using such immunoglobulin and/or antigen binding fragment(s) that specifically bind to PSD95 phosphorylated at threonine (19), at serine (25), and/or at both threonine (19) and serine (25).

### CROSS REFERENCE TO RELATED APPLICATIONS

This application claims the benefit of U.S. Provisional Application No. 63/358,178, filed Jul. 4, 2022 and U.S. Provisional Application No. 63/426,275, filed Nov. 17, 2022, which are incorporated by reference herein in their entirety.

### STATEMENT REGARDING FEDERALLY SPONSORED RESEARCH

This work was made with government support under National Institutes of Health, National Institutes of Health award 1ZIAAG000453. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jun. 30, 2023, is named “22-0813-WO_Sequence-Listing.xml” and is 82,852 bytes in size.

### BACKGROUND

### Field of the Disclosure

This disclosure generally relates to immunoglobulins and/or antigen binding fragments thereof that specifically bind threonine 19 phosphorylated PSD95, serine 25 phosphorylated PSD95, and/or PSD95 phosphorylated at both threonine 19 and serine 25 phosphorylated PSD95, corresponding expression vectors and host cells, and methods of use of such immunoglobulins and/or antigen binding fragments thereof.

### Description of Related Art

Postsynaptic density protein 95 (PSD95) belongs to the discs large (DLG) subfamily of the membrane-associated guanylate kinase (MAGUK) family. PSD95 interacts with both N-methyl-D-aspartate (NMDA) receptors and Shaker-type potassium channels and plays an important role in the formation and maintenance of synaptic junctions. PSD95 is a scaffolding protein that plays a central role in the genesis, maturation, stabilization, and function of synapses by providing a platform for clustering of key synaptic proteins, including glutamate receptors (NMDA, AMPA) and ApoE receptors (ApoER2). Structural integrity of PSD95 is crucial for shaping and strengthening synapses and regulating neuronal signaling cascades underlying memory, mood, and (hyper) excitability. Phosphorylation of PSD95 at Threonine 19 (to generate Thr19-phospho-PSD95) induces synapse disassembly. Excessive synapse disassembly is implicated in Alzheimer's disease, cognitive decline with aging, strokes, schizophrenia, depression, and anxiety. Failure to disassemble extraneous synapses is implicated in distractibility and hypersensitivity in autism-spectrum and attention deficit disorders.

Immunoglobulins and antigen binding fragments thereof specifically targeting Thr19-phospho-PSD95 are needed to better identify and diagnose neurological and psychiatric disorders characterized by excessive or inadequate synapse disassembly.

### SUMMARY

It is against the above background that the present disclosure provides certain advantages over the prior art.

Although this disclosure as provided herein is not limited to specific advantages or functionalities, the disclosure provides immunoglobulins and antigen binding fragments thereof that specifically bind Thr19-phospho-PSD95.

In one aspect, this disclosure provides an immunoglobulin or antigen binding fragment thereof that specifically binds postsynaptic density (PSD95) phosphorylated at threonine 19, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 41, the VH-CDR2 comprises the amino acid sequence set forth in SEQ ID NO: 42, the VH-CDR3 comprises the amino acid sequence set forth in SEQ ID NO: 43, the VL-CDR1 comprises the amino acid sequence set forth in SEQ ID NO: 46, the VL-CDR2 comprises the amino acid sequence set forth in SEQ ID NO: 47, and the VL-CDR3 comprises the amino acid sequence set forth in SEQ ID NO: 48. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 40. In certain embodiments, the VH comprises the amino acid sequence of SEQ ID NO: 40. In certain embodiments, the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 45. In certain embodiments, the VL comprises the amino acid sequence of SEQ ID NO: 45. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 40, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 45. In certain embodiments, the VH comprises the amino acid sequence set forth in SEQ ID NO: 40, and the VL comprises the amino acid sequence set forth in SEQ ID NO: 45. In some embodiments, the immunoglobulin or antigen binding fragment thereof is a monoclonal antibody.

In another aspect, this disclosure provides an immunoglobulin or antigen binding fragment thereof that specifically binds postsynaptic density (PSD95) phosphorylated at both threonine 19 and serine 25, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 53, the VH-CDR2 comprises the amino acid sequence set forth in SEQ ID NO: 54, the VH-CDR3 comprises the amino acid sequence set forth in SEQ ID NO: 55, the VL-CDR1 comprises the amino acid sequence set forth in SEQ ID NO: 58, the VL-CDR2 comprises the amino acid sequence set forth in SEQ ID NO: 47, and the VL-CDR3 comprises the amino acid sequence set forth in SEQ ID NO: 59. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 52. In certain embodiments, the VH comprises the amino acid sequence of SEQ ID NO: 52. In certain embodiments, the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 57. In certain embodiments, the VL comprises the amino acid sequence of SEQ ID NO: 57. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 52, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57. In certain embodiments, the VH comprises the amino acid sequence set forth in SEQ ID NO: 52, and the VL comprises the amino acid sequence set forth in SEQ ID NO: 57. In some embodiments, the immunoglobulin or antigen binding fragment thereof is a monoclonal antibody.

In another aspect, this disclosure provides an immunoglobulin or antigen binding fragment thereof that specifically binds postsynaptic density (PSD95) phosphorylated at both threonine 19 and serine 25, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 53, the VH-CDR2 comprises the amino acid sequence set forth in SEQ ID NO: 62, the VH-CDR3 comprises the amino acid sequence set forth in SEQ ID NO: 63, the VL-CDR1 comprises the amino acid sequence set forth in SEQ ID NO: 66, the VL-CDR2 comprises the amino acid sequence set forth in SEQ ID NO: 47, and the VL-CDR3 comprises the amino acid sequence set forth in SEQ ID NO: 59. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 61. In certain embodiments, the VH comprises the amino acid sequence of SEQ ID NO: 61. In certain embodiments, the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 65. In certain embodiments, the VL comprises the amino acid sequence of SEQ ID NO: 65. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 61, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 65. In certain embodiments, the VH comprises the amino acid sequence set forth in SEQ ID NO: 61, and the VL comprises the amino acid sequence set forth in SEQ ID NO: 65. In some embodiments, the immunoglobulin or antigen binding fragment thereof is a monoclonal antibody.

In some embodiments, the immunoglobulin or antigen binding fragment thereof as disclosed herein is an antibody fragment that binds to PSD95 phosphorylated at threonine 19 and/or serine 25.

In some embodiments, the immunoglobulin or antigen binding fragment thereof as disclosed herein binds to PSD-95 phosphorylated at threonine 19 and/or serine 25 with a dissociation constant (K_{d}) value of at least 10⁻⁸ M. In certain embodiments, the immunoglobulin or antigen binding fragment thereof is a monoclonal antibody.

In another aspect, this disclosure provides a nucleic acid, comprising a nucleotide sequence encoding the amino acid sequence of SEQ ID NO: 40 and 45.

In another aspect, this disclosure provides a nucleic acid, comprising a nucleotide sequence encoding the amino acid sequence of SEQ ID NO: 52 and 57.

In another aspect, this disclosure provides a nucleic acid, comprising a nucleotide sequence encoding the amino acid sequence of SEQ ID NO: 61 and 65.

In another aspect, this disclosure provides a vector comprising the nucleic acid as disclosed herein.

In another aspect, this disclosure provides a host cell comprising the expression vector as disclosed herein.

In another aspect, this disclosure provides an immunoglobulin or an antigen binding fragment that binds to protein in the postsynaptic density (PSD95) phosphorylated at threonine 19, at serine 25, and/or at both threonine 19 and serine 25. In certain embodiments, the immunoglobulin or an antigen binding fragment that binds to PSD95 phosphorylated at threonine 19 also binds to PSD95 phosphorylated at serine 25.

In another aspect, this disclosure provides, immunoglobulin or an antigen binding fragment that binds to protein in the postsynaptic density (PSD95) phosphorylated at threonine 19, at serine 25, and/or at both threonine 19 and serine 25, wherein the immunoglobulin or the antigen binding fragment is obtained by immunization with a polypeptide having an amino acid sequence selected from:

(SEQ ID NO: 1)
Ac-CDED(pT)PPLE;
(SEQ ID NO: 2)
CDED(pT)PPLE;
(SEQ ID NO: 3)
DED(pT)PPLE;
(SEQ ID NO: 4)
D(pT)PPLEHSP;
(SEQ ID NO: 5)
D(pT)PPLEH(pS)P;
(SEQ ID NO: 6)
D(pT)PPLEHSPA;
(SEQ ID NO: 7)
D(pT)PPLEH(pS)PA;
(SEQ ID NO: 8)
D(pT)PPLEHSPAH;
(SEQ ID NO: 9)
D(pT)PPLEH(pS)PAH;
(SEQ ID NO: 10)
ED(pT)PPLEHSPA;
(SEQ ID NO: 11)
ED(pT)PPLEH(pS)PA;
(SEQ ID NO: 12)
ED(pT)PPLEHSPAH;
(SEQ ID NO: 13)
ED(pT)PPLEH(pS)PAH;
(SEQ ID NO: 14)
DED(pT)PPLEHSPA;
(SEQ ID NO: 15)
DED(pT)PPLEH(pS)PA;
(SEQ ID NO: 16)
DED(pT)PPLEHSPAH;
(SEQ ID NO: 17)
DED(pT)PPLEH(pS)PAH;
(SEQ ID NO: 49)
Ac-CDED(pT)PPLEH(pS)PAH;
(SEQ ID NO: 50)
CDED(pT)PPLEH(pS)PAH;
(SEQ ID NO: 18)
KYRYQDED(pT)PPLEHSPAHLP;
(SEQ ID NO: 19)
KYRYQDED(pT)PPLEH(pS)PAHLP;
(SEQ ID NO: 20)
YRYQDED(pT)PPLEHSPAHLP;
(SEQ ID NO: 21)
YRYQDED(pT)PPLEH(pS)PAHLP;
(SEQ ID NO: 22)
RYQDED(pT)PPLEHSPAHLP;
(SEQ ID NO: 23)
RYQDED(pT)PPLEH(pS)PAHLP;
(SEQ ID NO: 24)
YQDED(pT)PPLEHSPAHLP;
(SEQ ID NO: 25)
YQDED(pT)PPLEH(pS)PAHLP;
(SEQ ID NO: 26)
QDED(pT)PPLEHSPAHLP;
(SEQ ID NO: 27)
QDED(pT)PPLEH(pS)PAHLP;
(SEQ ID NO: 28)
KYRYQDED(pT)PPLEHSPAHL;
(SEQ ID NO: 29)
KYRYQDED(pT)PPLEH(pS)PAHL;
(SEQ ID NO: 30)
KYRYQDED(pT)PPLEHSPAH;
(SEQ ID NO: 31)
KYRYQDED(pT)PPLEH(pS)PAH;
(SEQ ID NO: 32)
KYRYQDED(pT)PPLEHSPA;
(SEQ ID NO: 33)
KYRYQDED(pT)PPLEH(pS)PA;
(SEQ ID NO: 34)
KYRYQDED(pT)PPLEH(pS)P;
(SEQ ID NO: 35)
KYRYQDED(pT)PPLEHSP;
(SEQ ID NO: 36)
KYRYQDED(pT)PPLEHS;
(SEQ ID NO: 37)
KYRYQDED(pT)PPLEH(pS);
(SEQ ID NO: 38)
KYRYQDED(pT)PPLEH;

or an antigenic portion thereof.

In some embodiments, the immunoglobulin or the antigen binding fragment as disclosed herein comprise a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 41, or 53, the VH-CDR2 comprises the amino acid sequence set forth in SEQ ID NO: 42, 54, or 62, the VH-CDR3 comprises the amino acid sequence set forth in SEQ ID NO: 43, 55, or 63, the VL-CDR1 comprises the amino acid sequence set forth in SEQ ID NO: 46, 58, or 66, the VL-CDR2 comprises the amino acid sequence set forth in SEQ ID NO: 47, and the VL-CDR3 comprises the amino acid sequence set forth in SEQ ID NO: 48, or 59. In certain embodiments, the immunoglobulin or antigen binding fragment thereof comprises a VH comprising the amino acid sequence set forth in SEQ ID NO: 40, 52, or 61, and a VL comprising the amino acid sequence set forth in SEQ ID NO: 45, 57, or 65. In certain embodiments, the immunoglobulin or the antigen binding fragment is a monoclonal antibody.

In some embodiments, the immunoglobulin or the antigen binding fragments as disclosed herein are monoclonal antibodies. In some embodiments, the antigen binding fragment as disclose herein is an antibody fragment that binds to PSD95 phosphorylated at threonine 19. In some embodiments, the antigen binding fragment as disclose herein is an antibody fragment that binds to PSD95 phosphorylated at threonine 19 and serine 25. In some embodiments, the antigen binding fragment as disclose herein is an antibody fragment that binds to PSD95 phosphorylated at serine 25. In some embodiments, the immunoglobulin or the antigen binding fragments as disclosed herein bind to PSD95 phosphorylated at threonine 19 with a dissociation constant (K_{d}) value of at least 10⁻⁷ M.

In an aspect, this disclosure provides a nucleic acid comprising a nucleotide sequence encoding the immunoglobulin or an antigen binding fragments as disclosed herein.

In an aspect, this disclosure provides a vector comprising the nucleic acid encoding the immunoglobulin or an antigen binding fragments as disclosed herein.

In an aspect, this disclosure provides for host cells comprising the vector expressing the immunoglobulin or an antigen binding fragments as disclosed herein.

In an aspect, this disclosure provides methods of diagnosis of a neurological disorder in a subject using the immunoglobulin or antigen binding fragment as disclosed herein. In some embodiments, the diagnosis is in vitro and/or in vivo.

In an aspect, this disclosure provides methods for diagnosing a neurological disorder in a subject, comprising detecting in a sample from the subject threonine 19 phosphorylated PSD95, serine 25 phosphorylated PSD95, and/or both threonine 19 and serine 25 phosphorylated PSD95, wherein the sample is contacted with the immunoglobulin or antigen binding fragments as disclosed herein. In some embodiments, the diagnosis is in vitro and/or in vivo.

In certain embodiments, the method comprises an assay selected from the group consisting of: a radioimmunoassay, an immunohistochemistry assay, a competitive-binding assay, a Western Blot analysis, an ELISA assay, a two-dimensional gel electrophoresis, an enzyme immunoassay, a sandwich immunoassay, an immunodiffusion assay, an immunoradiometric assay, a fluorescent immunoassay, and an immunoelectrophoresis assay. In some embodiments, the antibodies as disclosed herein can be used to isolate a population of proteins that can then be assessed by proteomics.

In certain embodiments, the neurological disorder is selected from the group consisting of acute spinal cord injury, Alzheimer's disease, Amyotrophic Lateral Sclerosis (ALS), anxiety, ataxia, attention-deficit disorder, autism, behavior disorders (e.g., Attention Deficit Hyperactivity Disorder (ADHD), Behavioral Addiction, Conduct Disorder, Obsessive-compulsive disorder (OCD), and Oppositional Defiant Disorder (ODD)) Bell's palsy, brain tumors, cerebral aneurysm, cognitive decline, depression, epilepsy (and seizures), Guillain-Barre syndrome, headache, head injury, hydrocephalus, intellectual disorders (e.g., Down syndrome, fragile x syndrome, fetal alcohol syndrome, and Prader-Willi syndrome) meningitis, multiple sclerosis, muscular dystrophy, Parkinson's disease, schizophrenia, or stroke.

In certain embodiments, the sample is selected from the group consisting of blood, serum, plasma, urine, feces, respiratory secretions, exosome, cerebrospinal fluid, saliva, and brain tissue.

In an aspect, this disclosure provides methods of diagnosing and treating a neurological disorder in a subject, the method comprising:
(a) obtaining a sample from the subject;
(b) contacting the sample from the subject with an immunoglobulin or antigen binding fragment that specifically bind to threonine 19 phosphorylated PSD95, serine 25 phosphorylated PSD95, and/or both threonine 19 and serine 25 phosphorylated PSD95;
(c) detecting the presence or absence of threonine 19 phosphorylated PSD95, serine 25 phosphorylated PSD95, and/or both threonine 19 and serine 25 phosphorylated PSD95; or detecting the presence or absence of one or more complexes that include threonine 19 phosphorylated PSD95, serine 25 phosphorylated PSD95, and/or both threonine 19 and serine 25 phosphorylated PSD95 and an immunoglobulin or antigen binding fragment using a labeled secondary antibody;
(d) diagnosing the subject as having or as not having the neurological disorder based on the detection of the presence or absence of threonine 19 phosphorylated PSD95, serine 25 phosphorylated PSD95, and/or both threonine 19 and serine 25 phosphorylated PSD95; or the one or more complexes; and
(e) administering an effective treatment to treat the subject having the neurological disorder.

In certain embodiments, the methods further comprise detecting total PSD95 levels.

In certain embodiments, the immunoglobulin or the antigen binding fragment are obtained by immunization with a polypeptide having an amino acid sequence selected from:

(SEQ ID NO: 1)
Ac-CDED(pT)PPLE;
(SEQ ID NO: 2)
CDED(pT)PPLE;
(SEQ ID NO: 3)
DED(pT)PPLE;
(SEQ ID NO: 4)
D(pT)PPLEHSP;
(SEQ ID NO: 5)
D(pT)PPLEH(pS)P;
(SEQ ID NO: 6)
D(pT)PPLEHSPA;
(SEQ ID NO: 7)
D(pT)PPLEH(pS)PA;
(SEQ ID NO: 8)
D(pT)PPLEHSPAH;
(SEQ ID NO: 9)
D(pT)PPLEH(pS)PAH;
(SEQ ID NO: 10)
ED(pT)PPLEHSPA;
(SEQ ID NO: 11)
ED(pT)PPLEH(pS)PA;
(SEQ ID NO: 12)
ED(pT)PPLEHSPAH;
(SEQ ID NO: 13)
ED(pT)PPLEH(pS)PAH;
(SEQ ID NO: 14)
DED(pT)PPLEHSPA;
(SEQ ID NO: 15)
DED(pT)PPLEH(pS)PA;
(SEQ ID NO: 16)
DED(pT)PPLEHSPAH;
(SEQ ID NO: 17)
DED(pT)PPLEH(pS)PAH;
(SEQ ID NO: 49)
Ac-CDED(pT)PPLEH(pS)PAH;
(SEQ ID NO: 50)
CDED(pT)PPLEH(pS)PAH;
(SEQ ID NO: 18)
KYRYQDED(pT)PPLEHSPAHLP;
(SEQ ID NO: 19)
KYRYQDED(pT)PPLEH(pS)PAHLP;
(SEQ ID NO: 20)
YRYQDED(pT)PPLEHSPAHLP;
(SEQ ID NO: 21)
YRYQDED(pT)PPLEH(pS)PAHLP;
(SEQ ID NO: 22)
RYQDED(pT)PPLEHSPAHLP;
(SEQ ID NO: 23)
RYQDED(pT)PPLEH(pS)PAHLP;
(SEQ ID NO: 24)
YQDED(pT)PPLEHSPAHLP;
(SEQ ID NO: 25)
YQDED(pT)PPLEH(pS)PAHLP;
(SEQ ID NO: 26)
QDED(pT)PPLEHSPAHLP;
(SEQ ID NO: 27)
QDED(pT)PPLEH(pS)PAHLP;
(SEQ ID NO: 28)
KYRYQDED(pT)PPLEHSPAHL;
(SEQ ID NO: 29)
KYRYQDED(pT)PPLEH(pS)PAHL;
(SEQ ID NO: 30)
KYRYQDED(pT)PPLEHSPAH;
(SEQ ID NO: 31)
KYRYQDED(pT)PPLEH(pS)PAH;
(SEQ ID NO: 32)
KYRYQDED(pT)PPLEHSPA;
(SEQ ID NO: 33)
KYRYQDED(pT)PPLEH(pS)PA;
(SEQ ID NO: 34)
KYRYQDED(pT)PPLEH(pS)P;
(SEQ ID NO: 35)
KYRYQDED(pT)PPLEHSP;
(SEQ ID NO: 36)
KYRYQDED(pT)PPLEHS;
(SEQ ID NO: 37)
KYRYQDED(pT)PPLEH(pS);
(SEQ ID NO: 38)
KYRYQDED(pT)PPLEH;

or an antigenic portion thereof.

In certain embodiments, the one or more of the polypeptides for immunization as disclosed herein may be effective when given alone or in combination, or linked to, or fused to, another substance (for example acylated at the N-terminus, and/or include an N-terminal cysteine). In certain embodiments, the one or more of the polypeptides for immunization as disclosed herein may be administered at one time or over several intervals.

In certain embodiments, the immunoglobulin or the antigen binding fragment comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 41, or 53, the VH-CDR2 comprises the amino acid sequence set forth in SEQ ID NO: 42, 54, or 62, the VH-CDR3 comprises the amino acid sequence set forth in SEQ ID NO: 43, 55, or 63, the VL-CDR1 comprises the amino acid sequence set forth in SEQ ID NO: 46, 58, or 66, the VL-CDR2 comprises the amino acid sequence set forth in SEQ ID NO: 47, and the VL-CDR3 comprises the amino acid sequence set forth in SEQ ID NO: 48, or 59. In certain embodiments, the immunoglobulin or the antigen binding fragment comprises a VH comprising the amino acid sequence set forth in SEQ ID NO: 40, 52, or 61, and a VL comprising the amino acid sequence set forth in SEQ ID NO: 45, 57, or 65.

In certain embodiments, the immunoglobulin or the antigen binding fragment is labeled. In certain embodiments the immunoglobulin or the antigen binding fragment is immobilized on a solid support. In certain embodiments the solid support forms part of an enzyme-linked immunosorbent assay device. In certain embodiments the enzyme-linked immunosorbent assay device is a lateral flow immunoassay device.

In certain embodiments the neurological disorder is selected from the group consisting of acute spinal cord injury, Alzheimer's disease, Amyotrophic Lateral Sclerosis (ALS), anxiety, ataxia, attention-deficit disorder, autism, behavior disorders (e.g., Attention Deficit Hyperactivity Disorder (ADHD), Behavioral Addiction, Conduct Disorder, Obsessive-compulsive disorder (OCD), and Oppositional Defiant Disorder (ODD)) Bell's palsy, brain tumors, cerebral aneurysm, cognitive decline, depression, epilepsy (and seizures), Guillain-Barre syndrome, headache, head injury, hydrocephalus, intellectual disorders (e.g., Down syndrome, fragile x syndrome, fetal alcohol syndrome, and Prader-Willi syndrome) meningitis, multiple sclerosis, muscular dystrophy, Parkinson's disease, schizophrenia, or stroke.

In certain embodiments the sample is selected from the group consisting of blood, serum, plasma, urine, feces, respiratory secretions, exosome, cerebrospinal fluid, saliva, and brain tissue.

In another aspect, this disclosure provides kits for detecting PSD95 phosphorylated at threonine 19, serine 25, and/or PSD95 phosphorylated at both threonine 19 and serine 25 in a sample, the kit comprising: an immunoglobulin or an antigen binding fragment capable of binding specifically to PSD95 phosphorylated at threonine 19, serine 25, and/or PSD95 phosphorylated at both threonine 19 and serine 25 and a labeled immunoglobulin or a labeled antigen binding fragment immunoglobulin capable of binding specifically to the immunoglobulin or the antigen binding fragment capable of binding specifically to PSD95 phosphorylated at threonine 19, serine 25, and/or PSD95 phosphorylated at both threonine 19 and serine 25. In certain embodiments, the immunoglobulin or the antigen binding fragment of the kit is immobilized to a solid support.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present disclosure can be best understood when read in conjunction with the following drawings, where like structure is indicated with like reference numerals and in which:

FIG. 1 shows lipid peroxidation-induced ApoE/Reelin-ApoE receptor-Dab1 axis disruption as a unifying hypothesis underlying sporadic Alzheimer's disease. The ApoE cascade and Reelin cascade are depicted by light green and light blue shading, respectively. Aldehydic products of lipid peroxidation (orange stars) derived from lipid-loaded ApoE particles attack Lys and His-enriched sequences within ApoER2, VLDLR, and ApoE, generating lipid-protein adducts and crosslinked ApoE-ApoE receptor complexes. Crosslinks (FIG. 1A) and adducts (FIG. 1B) compromise ApoE receptor-ligand binding (FIG. 1C), leading to extracellular trapping of ApoE (FIG. 1D) and Reelin (FIG. 1E). Adducts and crosslinks disrupt ApoE-ApoER2 complex dissociation in early endosomes (FIG. 1F), thereby disrupting ApoER2 recycling (FIG. 1G), and delivery of the lipid cargo needed to remodel synaptic membranes (FIG. 1H). Impaired Reelin binding disrupts proteasomal degradation of Dab1, and lysosomal degradation of ApoER2, leading to localized accumulation of Dab1 (FIG. 1I) and ApoER2 (not shown). Ensuing compromise of the Reelin-Dab1-PI3K cascade destabilizes the actin cytoskeleton (FIG. 1J) and activates GSK3β by decreasing its phosphorylation (thin inhibition sign) (FIG. 1K). GSK3β-mediated phosphorylation of PSD95 and Tau destabilizes postsynaptic receptor complexes and the microtubule cytoskeleton, and promotes neurofibrillary tangle formation (FIG. 1L). Extracellular trapping of peroxidized ApoE (FIG. 1C) provides a nidus for Aβ deposition and oligomerization, leading to ApoE-Aβ plaque formation (FIG. 1K). Lysosomal accumulation of crosslinked lipid-protein polymers exacerbates proteinopathies (FIG. 1M). Collective derangements are proposed to account for the study findings and the defining hallmarks of AD (teal shading in FIG. 1L, FIG. 1K).

FIG. 2A-2C show Thr19-pPSD95 accumulation as evidence for synapse disassembly in sporadic AD. Coronal sections through the hippocampus and middle temporal gyrus from a representative (Braak stage VI, APOE3/3) sAD case (FIGURE. 2A₁-FIG. 2A₁₀) and a non-AD control case (FIG. 2A₁₁-FIG. 2A₁₅) were stained with antibodies against Thr19-pPSD95, a marker of post-synapse disassembly that is regulated by GSK3β. Thr19-pPSD95 was abundant in prominent globular structures (black arrows) and smaller fibrillar structures (open arrows) in the vicinity of neuritic plaques and surrounding abnormal neurons in the cornu ammonis (FIG. 2A₁-FIG. 2A₄), molecular layer of the dentate gyrus (FIG. 2A₅) and middle temporal gyrus (FIG. 2A₆-FIG. 2A₁₀) in AD cases with minimal or no expression in controls. Thr19-pPSD95 expression strongly correlated with histochemical progression and cognitive deficits (FIG. 2B₂-FIG. 2B₄, FIG. 2B₆-FIG. 2B₈). MPIHC revealed complex spatial and morphological relationships between Thr19-pPSD95 and other ApoE/Reelin-ApoER2 pathway components in the neuritic plaque niche (FIG. 2C₁-FIG. 2C₅), and substantial co-localization of Thr19-pPSD95 with MAP2 in swollen, dystrophic dendrites (yellow arrows in FIG. 2C₂, FIG. 2C₄-FIG. 2C₅). White arrows in FIG. 2C₂₋₅ show a MAP2-positive dendritic projection emanating from the DG into the ML. Red and blue boxed panels in FIG. 2A₁-FIG. 2A₁₀ and FIG. 2A₁₁-FIG. 2A₁₅ indicate sAD cases and non-AD controls, respectively. In FIG. 2B, open and closed blue circles indicate young controls and age-matched controls; open and closed red diamonds indicate MCI cases and sAD cases, respectively. Abbreviations: DG, dentate granule cells; ML, molecular layer; SP, stratum pyramidale; SR, stratum radiatum.

FIG. 3A-3B show convergence of ApoE/Aβ/Reelin-ApoER2-Dab1-PI3K-PLIMK1-Tau-PSD95 pathologies in the molecular layer of the dentate gyrus in sAD. Thr19-pPSD95 expression in the molecular layer of the dentate gyrus was increased in sAD cases, positively correlated with Braak stage and Aβ plaque load, and negatively correlated with MMSE scores (FIG. 3A₁₋₄). Single marker IHC (FIG. 3B₁) and MP-IHC (FIG. 3B₂₋₁₂) were used to label serial coronal sections of the dentate gyrus from the same sporadic AD case (Braak stage VI, APOE3/3) shown in FIG. 2A₁₋₅. Single marker IHC revealed prominent Thr19-pPSD95-positive globular structures (open arrows in FIG. 3B₁) amid numerous smaller fibrillary structures and discrete puncta in this region. MP-IHC revealed that these globular structures co-express Thr19-pPSD95 and MAP2 (arrows in FIG. 3B₂), suggesting localization to dystrophic dendrites. Colocalization of Ser202/Thr205-pTau (arrows in FIG. 3B₃₋₄) within a subset of these Thr19-pPSD95 and MAP2-positive globular structures is consistent with somatodendritic localization of Tau. Expression of ApoE, HNE-ApoE, and Reelin was primarily localized to extracellular plaques (depicted by stars in FIG. 3B₅₋₈). HNE-ApoE exhibited partial, incomplete colocalization with native ApoE in the molecular layer of the dentate gyrus (FIG. 3B₆₋₈). Panels FIG. 3B₅₋₈ reveal partial co-localization of HNE-ApoE and Thr19-pPSD95 within swollen dendrites (yellow arrows) that surround BAP and ApoE enriched plaques (white stars). Multiplex and single-marker IHC revealed that ApoER2 LA1-2 expression in this region was localized to fibrillar structures and discrete puncta (FIG. 3B₇₋₉) in the immediate vicinity of neuritic plaques, while single-marker IHC revealed localized accumulation of Dab1, and strong expression of Tyr607-pPI3K and Thr508-pLIMK1 in unspecified globular structures that resemble dystrophic neurites. In FIG. 3A, open and closed blue circles indicate young controls and age-matched controls; open and closed red diamonds indicate MCI cases and sAD cases, respectively. Abbreviations: DG, dentate granule cells; ML, molecular layer.

FIG. 3C shows extracellular ApoE, HNE-ApoE and Reelin converge with Thr19-pPSD95 in dendritic arbors extending into the molecular layer of the dentate gyrus. Panels A and B are images of the hippocampus from a representative sporadic AD case (Braak stage V, ApoE3/3) selected to illustrate the local environment around a representative Aβ and ApoE-enriched neuritic plaque using MP-IHC. Panels C-L are enlarged images of a single representative plaque in A-B. Panels C-D illustrate prominent tortuosity of the MAP2-labeled dendritic molecular layer projections of dentate gyrus neurons, but minimal colocalization with Aβ in the vicinity of neuritic plaques. Panels D-F reveal striking colocalization (open arrows) of MAP2 (green, D) with Thr19-pPSD95 (red, E) (marker of postsynaptic disassembly) within these tortuous dendritic projections. Panels G-I reveal close relations but minimal colocalization between axons (NFL [green, G, I]) and Reelin (red, H, I). Panels J-L reveal HNE-modified ApoE (HNE-ApoE [green, J, L]) immunoreactive puncta (white arrows) within the plaque core with only partial co-localization with native ApoE (red, K-L).

FIG. 4 shows that Thr19-phospho-PSD95 is highly and selectively expressed by affected neurons and neuritic plaques in the entorhinal-hippocampal memory system in Alzheimer's disease [M280 Clone 1 & Clone 2].

FIG. 5 shows that Thr19-phospho-PSD95 is highly expressed in affected neurons and neuritic plaques within the cornu ammonis in Alzheimer's disease. [M280 Clone 1].

FIG. 6 shows that Thr19-phospho-PSD95 is highly expressed in affected neurons in Alzheimer's disease [M280 Clone 1].

FIG. 7 shows that Thr19-phospho-PSD95 is highly expressed in neuritic plaques in Alzheimer's disease [M280 Clone 1].

FIG. 8A-8B show diffuse plaques are enriched in Aβ but lack the ApoE and Thr19-pPSD95 that are abundant in neuritic plaques. Unlike Aβ, virtually all ApoE-immunoreactive plaques are associated with dystrophic neurites. FIG. 8A₁₋₅ show a coronal section of hippocampus from Mild Cognitive Impairment case (Braak Stage IV, ApoE 2/3) that had extensive diffuse Aβ plaques (total brain Aβ plaque load=13.5 out of 15) but only mild cognitive deficits (MMSE 26). FIG. 8B₁₋₁₀ are a coronal section from a representative sAD case (Braak stage V, ApoE3/3) with extensive neuritic plaques (total brain Aβ plaque load=15 out of 15) and major cognitive deficits (MMSE 6). Sections were stained with an antibody against Aβ (MOAB2, red), the ApoE C-terminal region (green), and Thr19-pPSD95 (cyan), with NEUN staining of neuronal nuclei shown in blue. Diffuse plaques in the MCI case had minimal expression of ApoE and Thr19-pPSD95 (white arrows in A₅). By contrast, the neuritic plaques in the sAD case had high expression of ApoE and Aβ in the plaque core (yellow arrows in B₅), and extensive accumulation of Thr19-pPSD95 in dystrophic neurons (closed white arrows in B₅₋₁₀) and surrounding abnormal neurons (open white arrows in Bₑ) in the vicinity of ApoE-enriched plaques.

FIG. 9A-9G show diffuse IHC evidence for convergence of ApoE/Reelin-ApoER2-Thr19-pPSD95 axis pathologies in the perforant path target zones in early sAD. Single-marker IHC was used to label serial coronal sections of the perforant path target zone from early sporadic AD case (MMSE 24, Braak stage V, APOE3/3). FIG. 9A shows ApoER2 expression within dentate granule cells and pyramidal neurons in CA1-3. FIG. 9B shows expression of Thr19-pPSD95 (marker of synaptic disassembly) in neuritic plaques and abnormal neurons in CA1-2, and their dendritic projections emanating into the SR subfield. FIG. 9C₁₋₅ reveal plaque associated Reelin accumulation in the molecular layer of the dentate gyrus (FIG. 9C₁₋₃, arrows) and the SP layer of CA2 (FIG. 9C₄₋₅, arrows). FIG. 9D1-5 reveal plaque associated ApoE accumulation in the molecular layer of the dentate gyrus (FIG. 9D₁₋₃) and the SLM subfield of CA2 (FIG. 9D₄₋₅). FIG. 9E₁₋₅ reveal plaque associated ApoER2 LA1-2 aggregates in the molecular layer of the dentate gyrus (FIG. 9E₁₋₃, arrows) and the SR and SP subfields of CA1-2 (FIG. 9E₄₋₅, arrows) FIG. 9F₁₋₅ reveal plaque associated Dab1 accumulation that is most pronounced in the SP and SR subregions of CA2 and CA1 (FIG. 9F₁₋₅, arrows). FIG. 9G₁₋₅ reveal strong Thr19-pPSD95 expression in neuritic plaques, affected neurons and their projections in CA1 and CA2 (FIG. 9G₁₋₃). FIG. 9G₄₋₅ show strong expression in Thr19-pPSD95 in neuritic plaque complexes within SR and SLM of CA1. Abbreviations: DG, dentate granule cells; ML, molecular layer; CA, cornu ammonis; SP, stratum pyramidale; SR, stratum radiatum; SLM, stratum lacunosum-moleculare.

FIG. 10 shows a table with associations between immunohistochemical markers and ante-mortem cognitive performance and neuropathologic endpoints. ^{a} Spearman correlations have three levels of shading based on p-values: light, 0.05≥p>0.01; moderate, 0.01≥p≥0.001; dark, p<0.001. ^{b} Folstein Mini Mental State Examination score (0-30) obtained most proximal to death; includes MMSE scores obtained through BBDP research clinical visits and by review of private medical records. ^{c} Braak stage describing topographical progression of neurofibrillary tangles, dystrophic neurites and neuropil threads, throughout transentorhinal and entorhinal areas, CA1 subfield of hippocampus, amygdala and cerebral neocortex. Evaluations were made, similarly as the original publication, 63 in large (3 cm×5 cm) thick (40 or 80 μm) sections stained with the Campbell-Switzer silver stain, Gallyas silver stain and Thioflavin S stains. Final judgment of tangle density is made on the basis of combined impression from all three stains. For three years, all cases were also stained with the AT8 antibody for phosphorylated tau protein. Note that the AT8 stain has been reported to give higher Braak stages as more neurites are apparent. ^{d} Average neurofibrillary tangle density in the cortex of the frontal lobe, including superior, middle, and inferior frontal gyri; cortex of the temporal lobe; cortex of the parietal lobe; CA1 subfield of hippocampus; and entorhinal cortex. Tangle density scored according to the CERAD templates, 53 as described for the Braak stage above. ^{e} Average senile (amyloid) plaque density (all types of plaques considered together) in the cortex of frontal lobe, including superior, middle, and inferior frontal gyri; cortex of temporal lobe; cortex of parietal lobe; CA1 subfield of hippocampus; and entorhinal cortex. Plaque density scored according to CERAD templates, 53 using large (3 cm×5 cm) thick (40 or 80 μm) sections stained with Campbell-Switzer and Gallyas silver stains, and Thioflavin S stains. Validity and accuracy of this combination for estimating density of Aβ deposits established in BBDP laboratories through strong correlations with autoradiographic binding of Florbetapir (amyloid imaging ligand), with biochemical measures (ELISA) of Aβ in human cerebral cortex extracts and with quantitative measures (percentage of section area occupied) of an immunohistochemical stain for Aβ. ^{f} Greatest neuritic plaque density observed across frontal, temporal and parietal cortex regions, scored according to CERAD templates.53 Evaluations made in large (3 cm×5 cm) thick (40 or 80 μm) sections stained with the Campbell-Switzer silver stain, Gallyas silver stain and Thioflavin S stains. Final judgment of plaque density made on the basis of the combined impression from all three stain.

FIG. 11 shows the Clinical, neuropathological, and cognitive characteristics of age matched Alzheimer's disease, MCI, and Control specimens (n=24).

FIG. 12 shows twenty-nine cases spanning the clinicopathological spectrum of Alzheimer's disease progression.

FIG. 13 shows the demographic, neuropathological, and cognitive characteristics of twelve cases spanning the clinicopathological spectrum of Alzheimer's disease progression (pPSD95).

FIG. 14 shows that the M280-3H4 clone and subclones selectively detect Thr19-phosphorylated PSD95. ELISA assays showed that mouse IgG1 monoclonal antibodies generated from parent clone 3H4 and several subclones including 3H4.F6 and 3H4.H6 selectively detected Thr19-phosphorylated PSD95 with minimal or no detection of the corresponding non-phosphorylated PSD95 sequence or a BSA control. Each clone and subclone had >30-fold higher affinity for the Thr19-phosphorylated PSD95 compared to controls. Coating antigen concentration: 2 ug/mL. Blocking buffer: 0.1% BSA-PBS. Primary antibody: fusion supernatant (neat). Secondary antibody: Goat anti-Mouse IgG at 1:10,000 dilution.

FIG. 15A-15D show that the M280-3H4.F6 monoclonal detects Thr19-phosphorylated PSD95 in blood and brain with higher sensitivity than commercial polyclonal PSD95 antibodies. Example chromatographs from top-down HPLC/MRM targeted mass spectrometry using heavy isotope standards positively identified and quantitated Thr19pPSD95 from M280-H4.F6 monoclonal antibody immune-precipitated blood of (A) control serum and (B) Alzheimer's disease serum with (C) +4-times higher levels from Alzheimer's disease serum (C) and brain (D) samples compared to control specimens. M280-H4.F6 had higher sensitivity for detection of Thr19-pSPD95 than commercially available antibodies in blood and brain.

FIG. 16A-16C show that HPLC/MS confirmation that M280-H4.F6 monoclonal antibody detects Thr19-phosphorylated PSD95 in Alzheimer's disease blood. FIG. 16A) bottom-up “untargeted” HPLC/MS chromatogram of all proteins pulled down from immuno-precipitation assay of Alzheimer's disease blood serum sample using M280-H4.F6 monoclonal antibody followed by full proteinase K digestion into peptides. FIG. 16B) Top-5 peptide ion peaks from mass spectrometry scan confirmed Thr19pPSD95 as the majority protein present from the antibody pull-down (peptide ions mz=588.3, 570.3, and 776.4) with 40/49 of all predicted peptides from proteinase K digest being identified. This analysis also found the presence of heterometric protein complex/s being pulled down with Thr19pPSD95 that includes a strong signature profile for pTau (peptide ions mz=654.4 and 804.4). FIG. 16C) MS1 presents of Thr19pPSD95 peptide ion mz=994.3 at standardized retention time and MS2/3 daughter ion profiles further supports MRM findings in FIG. 15.

FIG. 17A-17C show that monoclonal antibody (M280-H4.F6) selectively labels Thr19-pPSD95-enriched neuritic plaques and abnormal neurons in Alzheimer's disease. Panel A depicts Thr19-pPSD95 expression in the hippocampus and medial temporal lobe memory system. Panel B depicts Thr19-pPSD95 expression in the cornu ammonis (CA) 1-2 region of the hippocampus. Panel C shows that Thr19-pPSD95 is highly expressed in swollen, dystrophic neurites localized to neuritic plaques.

FIG. 18 shows that hippocampal Thr19-pPSD95 expression increases across the spectrum of Alzheimer's disease (AD). Immunolabeling with M280-H4.F6 monoclonal antibody in a representative Braak stage II non-AD control (Panels 1-5) shows that Thr19-pPSD95 expression is limited to prominent granulovacuolar structures that resemble granulovacuolar degeneration bodies in a small subset of abnormal neurons. A representative Braak stage III control (Panels 6-10) demonstrates more pronounced Thr19-pPSD95 expression in a larger subset of abnormal neurons and their dendritic arbors (open arrow in Panel 8) and granulovacuolar degeneration bodies (arrows in Panel 10). A representative Braak stage IV AD case (Panels 11-15) demonstrates more pronounced Thr19-pPSD95 expression in abnormal neurons, neurites, and dystrophic neuritic components of neuritic plaques (star in Panel 13). Arrows in panels 14-15 depicts prominent peri-nuclear vacuolar immunostaining that is minimal or absent in non-AD cases.

FIG. 19 shows that M280-H4. F6 monoclonal antibody labels Thr19-pPSD95-enriched abnormal neurons in layer II of the entorhinal cortex in early (preclinical) stages of Alzheimer's disease. Open arrows in panels 1-2 and 6-7 depict Thr19-pPSD95 expression in entorhinal cortex layer II (L2) neurons and their dense dendritic arbors emanating into layer I (L1). Arrows in panels 3-5 and 9-10 demarcate Thr19-pPSD95 expression in discrete puncta surrounding abnormal neurons.

FIG. 20 shows that Thr19-pPSD95 expression in the subiculum increases across the clinicopathological spectrum of Alzheimer's disease. Immunolabeling with monoclonal antibody (M280-H4.F6) in representative cases with severe advanced AD (Braak stage VI, Panels 1-5) and Braak stage V AD (Panels 6-10) demonstrate prominent expression of Thr19-pPSD95 expression in dystrophic neurites, especially in the vicinity of neuritic plaques (stars in Panels 3-4), and in granulovacuolar structures that resemble granulovacuolar degeneration bodies within affected neurons (open arrows in Panels 5 and 9-10). A representative Braak stage III control (Panels 11-15) demonstrates Thr19-pPSD95 expression in a subset of abnormal neurons that is mostly restricted to the nucleus (closed arrows in 14-15), with vacuolar staining evident in a small subset of dendritic projections (open arrows Panel 14). A representative Braak stage I control (Panels 16-20) demonstrates rare granulovacuolar Thr19-pPSD95 expression that is restricted to the cytoplasm of a very small subset of affected neurons (open arrows in Panels 18-20).

FIG. 21 shows that M280-3H4 monoclonal antibodies selectively label bands consistent with Thr19-pPSD95 in human brain and PSD95 overexpression lysates. M280 3H4.F6 & 3H4.H6 monoclonal antibodies detect bands consistent with PSD95 monomers and multimers in human brain (Lanes 4-5) with minimal or no detection in human skeletal muscle and liver (Lanes 2-3). M280 3H4.F6 & 3H4.H6 monoclonal antibodies also detect a 95 kDa band consistent with PSD95 monomer in the PSD95 overexpression lysate but do not detect recombinant (non-phosphorylated) PSD95. This lack of detection in non-phosphorylated recombinant PSD95 protein (Lane 6) differentiates M280-3H4 monoclonals from a less selective commercially available Thr19-pPSD95 rabbit polyclonal antibody that also detects non-phosphorylated recombinant PSD95 protein (see FIG. 23).

FIG. 22 shows that M280-3H4 monoclonal antibody detection in human AD brain is completely blocked by pre-incubation with Thr19-pPSD95 containing blocking peptides. M280-3H4.F6 & M280-3H4. H6 monoclonal antibodies detect bands consistent with PSD95 monomers and multimers in human brain (Lanes A2 and D2). Pre-incubation with either of two peptides containing the Thr19-pPSD95 sequence completely blocked PSD95 detection (Lanes B2-C2 and E2-F2).

FIG. 23 shows that polyclonal antibody targeting Thr19-pPSD95 has residual reactivity toward non-phosphorylated PSD95 protein. A commercially available antibody targeting Thr19-pPSD95 (Millipore-ABN998) detects bands consistent with PSD95 in human AD brain (Lanes 4-5) with minimal or no detection in non-brain tissues (Lanes 2-4). Unlike the M280-3H4 monoclonals, this polyclonal antibody has residual reactivity toward recombinant (non- phosphorylated) PSD95 protein (Lane 7).

FIG. 24 shows that immunoprecipitation with M280-3H4 monoclonal antibodies isolates high molecular weight protein complexes in blood from human AD cases. Immunoprecipitation of serum of AD cases with the M280-3H4.F6 monoclonal antibody isolated proteins or protein complexes with molecular weights of ≈280 and ≈370 kDa. These molecular weights are ≈3 to 4 times as high as monomeric PSD95 (90-95 kDa) suggesting that they are either Thr19-pPSD95 trimers and tetramers or Thr19-pPSD95-containing heteromeric protein complexes. This interpretation is consistent with knowledge that PSD95 is a scaffolding protein that is known to polymerize and to interact with and organize numerous dendritic proteins, and mass spectrometry data from the same M280-3H4.F6 immunoprecipitation material which showed strong signatures for Thr19-pPSD95 and the presence of other proteins that are known to interact with PSD95. *Millipore-ABN998 Rabbit polyclonal antibody targeting Thr19-pPSD95.

FIG. 25 shows that use of ELISA to quantify Thr19-pPSD95 in Alzheimer's disease and control plasma. ELISA results using Thr19pPSD95 peptide conjugated to BSA coated plates and as competing antigen for M280-H4.F6 antibody showed an almost 30-fold increased presents of competing antigens in Alzheimer's disease sample (diamond) Vs. control (triangle).

FIG. 26 shows that M312-E1 and M312-10F3 clones and subclones selectively detect dual phosphorylated (Thr19+Ser25) PSD95. ELISA assays showed that mouse IgG1 monoclonal antibodies generated from parent clones 3E1 and 10F3 and several subclones including M312-3E1.D7 and M312-10F3.H2 selectively detected dual phosphorylated (Thr19+Ser25) PSD95 with minimal or no detection of the corresponding single phosphorylated (Ser25) sequence, non-phosphorylated PSD95 sequences, or a BSA control. Each clone and subclone had >13-fold higher affinity for the Thr19-phosphorylated PSD95 compared to both controls. M312-3E2 detected both dual phosphorylated (Thr19+Ser25) PSD95 and the Ser25 phosphorylated PSD95 but not the control. Coating antigen concentration: 2ug/mL. Blocking buffer: 0.1% BSA-PBS. Primary antibody: fusion supernatant (neat). Secondary antibody: Goat anti-Mouse IgG at 1:10,000 dilution.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present disclosure.

### DETAILED DESCRIPTION OF THE DISCLOSURE

All publications, patents and patent applications cited herein are hereby expressly incorporated by reference for all purposes.

Before describing the present disclosure in detail, a number of terms will be defined. Unless otherwise required by context, singular terms shall include pluralities and plural terms shall include the singular. For example, the singular forms “a,” “an,” and “the” include plural referents unless the context clearly dictates otherwise.

It is noted that terms like “preferably,” “commonly,” and “typically” are not utilized herein to limit the scope of the claimed subject matter or to imply that certain features are critical, essential, or even important to the structure or function of the claimed subject matter. Rather, these terms are merely intended to highlight alternative or additional features that can or cannot be utilized in a particular embodiment of the present disclosure.

For the purposes of describing and defining the present disclosure it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this disclosure. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

In some embodiments provided herein is an immunoglobulin or an antigen binding fragment that binds to postsynaptic density (PSD95) phosphorylated at threonine 19. In some embodiments provided herein is an immunoglobulin or an antigen binding fragment that binds to postsynaptic density (PSD95) phosphorylated at both threonine 19 and serine 25. In some embodiments provided herein is an immunoglobulin or an antigen binding fragment that binds to postsynaptic density (PSD95) phosphorylated at serine 25.

In some embodiments, the immunoglobulin or the antigen binding fragment is a monoclonal antibody. In certain embodiments, the monoclonal antibody binds to PSD95 phosphorylated at threonine 19. In certain embodiments, the monoclonal antibody binds to PSD95 phosphorylated at both threonine 19 and serine 25. In certain embodiments, the monoclonal antibody binds to PSD95 phosphorylated at serine 25.

The terms “binds” or “specifically binds” refers to an immunoglobulin or an antigen binding fragment that binds to a molecule or a fragment thereof of PSD95 that is phosphorylated at threonine 19. Binding or specific binding between two entities, may include a binding affinity of at least 10⁶, 10⁷, 10⁸, 10⁹, 10¹⁰, or 10¹¹ M⁻¹. Affinities greater than 10⁷ M⁻¹ are preferred for specific binding. The immunoglobulin or the antigen binding fragment that specifically binds a molecule or a fragment thereof can bind to other molecules with lower affinity as determined by, e.g., immunoassays, BIAcore, or other assays known in the art.

In certain embodiments, the immunoglobulin or the antigen binding fragment specifically binds to post-synaptic density protein 95. Postsynaptic density protein 95 (PSD95) is also known as discs large MAGUK scaffold protein 4 (DLG4; as well as gene synonyms MRD62; PSD95; SAP-90; and SAP90). DLG4 belongs to the discs large (DLG) subfamily of the membrane-associated guanylate kinase (MAGUK) family. PSD95 interacts with both N-methyl-D-aspartate (NMDA) receptors and Shaker-type potassium channels and plays an important role in the formation and maintenance of synaptic junctions. PSD95 is also a regulator of synaptic maturation by interacting, stabilizing and trafficking N-methyl-D-aspartic acid receptors (NMDARs) and α-amino-3-hydroxy-5-methyl-4-isox-azoleproprionic acid receptors (AMPARs) to the postsynaptic membrane. Evidence associates PSD95 disruption with cognitive deficits. There are at least two recognized isoforms of PSD95. PSD isoform 1 and PSD isoform 2. PSD isoform 2 includes an additional 43 amino acids at the N-terminal.

In particular embodiments, the immunoglobulin or the antigen binding fragment specifically binds to post-synaptic density protein 95 phosphorylated at threonine 19. In some embodiments, the immunoglobulin or the antigen binding fragment specifically binds to post-synaptic density protein 95 phosphorylated at serine 25. In some embodiments, the immunoglobulin or the antigen binding fragment specifically binds to post-synaptic density protein 95 phosphorylated at both threonine 19 and serine 25. In certain embodiments, the PSD95 can refer to PSD95 isoform 1 (also known as PSD95 alpha), thus in certain embodiments, the immunoglobulin or an antigen binding fragments disclosed herein bind to PSD95 isoform 1 (PSD alpha) phosphorylated at threonine 19. In certain embodiments, the immunoglobulin or an antigen binding fragments disclosed herein bind to PSD95 isoform 1 (PSD alpha) phosphorylated at threonine 19 and at serine 25. In certain embodiments, the immunoglobulin or an antigen binding fragments disclosed herein bind to PSD95 isoform 1 (PSD alpha) phosphorylated at serine 25. In some embodiments, the PSD95 can refer to PSD95 isoform 2 (also known as PSD95-beta), thus in certain embodiments, the immunoglobulin or an antigen binding fragments disclosed herein bind to PSD95 isoform 2 (PSD beta) phosphorylated at threonine 62. In certain embodiments, the immunoglobulin or an antigen binding fragments disclosed herein bind to PSD95 isoform 2 (PSD beta) phosphorylated at threonine 62 and at serine 68. In certain embodiments, the immunoglobulin or an antigen binding fragments disclosed herein bind to PSD95 isoform 2 (PSD beta) phosphorylated at serine 68.

PSD95 beta has different regional and cellular distribution than PSD95 alpha and its loss is implicated in neurological diseases including schizophrenia.

Some aspects of the present disclosure are directed to immunoglobulins or antigen binding fragments thereof that specifically bind post-synaptic density protein 95 phosphorylated at threonine 19. Some aspects of the present disclosure are directed to immunoglobulins or antigen binding fragments thereof that specifically bind post-synaptic density protein 95 phosphorylated at serine 25. In certain embodiments, the immunoglobulin or an antigen binding fragment that binds to PSD95 phosphorylated at threonine 19 also binds to PSD95 phosphorylated at serine 25. In some embodiments, the immunoglobulins or antigen binding fragments thereof comprise a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3.

In some embodiments, the VH-CDR1 comprises the amino acid sequence set forth in SEQ ID NO: 41, the VH-CDR2 comprises the amino acid sequence set forth in SEQ ID NO: 42, the VH-CDR3 comprises the amino acid sequence set forth in SEQ ID NO: 43, and the VL-CDR1 comprises the amino acid sequence set forth in SEQ ID NO: 46, the VL-CDR2 comprises the amino acid sequence set forth in SEQ ID NO: 47, and the VL-CDR3 comprises the amino acid sequence set forth in SEQ ID NO: 48. In some embodiments, the immunoglobulin or antigen binding fragment thereof comprise a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 40. In some embodiments, the VH comprises the amino acid sequence of SEQ ID NO: 40. In certain embodiments, the immunoglobulin or antigen binding fragment thereof comprises a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 45. In some embodiments, the VL comprises the amino acid sequence of SEQ ID NO: 45. In certain embodiments, the VH comprises the amino acid sequence set forth in SEQ ID NO: 40, and the VL comprises the amino acid sequence set forth in SEQ ID NO: 45.

In some embodiments, the VH-CDR1 comprises the amino acid sequence set forth in SEQ ID NO: 53, the VH-CDR2 comprises the amino acid sequence set forth in SEQ ID NO: 54, the VH-CDR3 comprises the amino acid sequence set forth in SEQ ID NO: 55, the VL-CDR1 comprises the amino acid sequence set forth in SEQ ID NO: 58, the VL-CDR2 comprises the amino acid sequence set forth in SEQ ID NO: 47, and the VL-CDR3 comprises the amino acid sequence set forth in SEQ ID NO: 59. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 52. In certain embodiments, the VH comprises the amino acid sequence of SEQ ID NO: 52. In certain embodiments, the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 57. In certain embodiments, the VL comprises the amino acid sequence of SEQ ID NO: 57. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 52, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57. In certain embodiments, the VH comprises the amino acid sequence set forth in SEQ ID NO: 52, and the VL comprises the amino acid sequence set forth in SEQ ID NO: 57. In some embodiments, the immunoglobulin or antigen binding fragment thereof is a monoclonal antibody.

In some embodiments, the VH-CDR1 comprises the amino acid sequence set forth in SEQ ID NO: 53, the VH-CDR2 comprises the amino acid sequence set forth in SEQ ID NO: 62, the VH-CDR3 comprises the amino acid sequence set forth in SEQ ID NO: 63, the VL-CDR1 comprises the amino acid sequence set forth in SEQ ID NO: 66, the VL-CDR2 comprises the amino acid sequence set forth in SEQ ID NO: 47, and the VL-CDR3 comprises the amino acid sequence set forth in SEQ ID NO: 59. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 61. In certain embodiments, the VH comprises the amino acid sequence of SEQ ID NO: 61. In certain embodiments, the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 65. In certain embodiments, the VL comprises the amino acid sequence of SEQ ID NO: 65. In certain embodiments, the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 61, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 65. In certain embodiments, the VH comprises the amino acid sequence set forth in SEQ ID NO: 61, and the VL comprises the amino acid sequence set forth in SEQ ID NO: 65. In some embodiments, the immunoglobulin or antigen binding fragment thereof is a monoclonal antibody.

As used herein, the terms “immunoglobulin” and “antigen-binding fragment thereof” refer to at least the minimal portion of an immunoglobulin and/or antibody which is capable of binding to a specified antigen which the antibody targets, e.g., at least some of the complementarity determining regions (CDRs) of the variable domain of a heavy chain (VH) and the variable domain of a light chain (VL) in the context of a typical antibody produced by a B cell. The VH and VL regions can be subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. Unless specified otherwise herein, the amino acids in the variable regions are numbered using the Kabat numbering system and those in the constant regions are numbered using the EU system.

Immunoglobulins or antigen binding fragments thereof as disclosed herein can be or be derived from polyclonal, monoclonal, human, humanized, or chimeric antibodies, single chain antibodies, epitope-binding fragments, e.g., a Fab, Fab′ and F(ab′)2, Fd, Fv, single-chain Fv (scFv), single-chain antibody, disulfide-linked Fv (sdFv), or fragments comprising either a VL or VH domain alone or in conjunction with a portion of the opposite domain (e.g., a whole VL domain and a partial VH domain with one, two, or three CDRs), and fragments produced by a Fab expression library. Antibody molecules encompassed by this disclosure can be of or be derived from any type (e.g., IgG, IgE, IgM, IgD, IgA, and IgY), class (e.g., IgG1, IgG2, IgG3, IgG4, IgA1 and IgA2), or subclass of immunoglobulin molecule.

The term “antigen binding fragment” of an antibody, as used herein, refers to one or more fragments of an antibody that retain the ability to specifically bind to an antigen (e.g., human PSD95). The antigen-binding function of an antibody can be performed by fragments of a full-length antibody. Examples of binding fragments encompassed within the term “antigen-binding portion” of an antibody, include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

In certain embodiments, the immunoglobulins or antigen binding fragments as described herein can include chimeric antibodies. The term “chimeric antibody” refers to a recombinant antibody or to an engineered antibody which in its broadest sense contains one or more regions from one antibody and one or more regions from one or more other antibody(ies). In some embodiments, a chimeric antibody comprises a VH domain and a VL domain of an antibody derived from a non-human animal, in association with a CH domain and a CL domain of another antibody, in particular a human antibody. As the non-human animal, any animal such as camel, llama, mouse, rat, hamster, rabbit or the like can be used. A chimeric antibody may also denote a multi-specific antibody having specificity for at least two different antigens.

In certain embodiments, the immunoglobulins or antigen binding fragments as described herein can include humanized antibodies. The term “humanized antibody” refers to an antibody which is wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains.

Numerous methods for humanization of an antibody sequence are known in the art; see e.g., the review by Almagro & Fransson (2008) Front Biosci. 13: 1619-1633. One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues. A further method of humanization is the so-called 4D humanization. The 4D humanization protocol is described in WO 2009/032661A1, which is incorporated by reference herein in its entirety.

The term “K_{D},” as used herein, refers to the dissociation constant of the interaction between the immunoglobulin or the antigen binding fragment disclosed herein and a target antigen. In some embodiments, the immunoglobulin or the antigen binding fragment binds to post-synaptic density protein 95 phosphorylated at threonine 19 or PSD95 phosphorylated at both threonine 19 and serine 25, with a dissociation constant (K_{D}) of 10⁻⁵ to 10⁻¹² moles/liter or less, or 10⁻⁷ to 10⁻¹² moles/liter or less, or 10⁻³ to 10⁻¹² moles/liter or less. In some embodiments, the immunoglobulin or the antigen binding fragment thereof binds to PSD95 phosphorylated at threonine 19 or PSD95 phosphorylated at both threonine 19 and serine 25, with a K_{D} of about 1.5E-9 to about 2.5E-9. In some embodiments, the immunoglobulin or the antigen binding fragment thereof binds to post-synaptic density protein 95 phosphorylated at threonine 19 with a binding affinity of at least 10⁷ M⁻¹, or at least 10⁸ M⁻¹, or at least 10⁹ M⁻¹, or at least 10¹² M⁻¹. In some embodiments, the immunoglobulin or the antigen binding fragments of the disclosure will bind to a desired antigen of PSD95 with an affinity less than 500 mM, or less than 200 nM, or less than 10 nM, or less than 500 pM.

TABLE 1
Kd Determination (BLI method).

| Clone | K_{d} (M) | Kₒₙ (1 Ms) | K_{off} (1/s) |
|---|---|---|---|
| 3H4.F65 | 1.63E−09 | 8.97E+04 | 1.46E−04 |
| 3H4.H6 | 2.23E−09 | 7.47E+04 | 1.67E−04 |

The dissociation constant (K_{D}) can be determined, for example, by surface plasmon resonance (SPR). Generally, surface plasmon resonance analysis measures real-time binding interactions between a ligand (a target antigen on a biosensor matrix) and an analyte by surface plasmon resonance using, for example, the BIAcore system (Pharmacia Biosensor; Piscataway, NJ). Surface plasmon analysis can also be performed by immobilizing the analyte and presenting the ligand. Specific binding of the immunoglobulin or the antigen binding fragment that specifically binds PSD95 can also be determined in any suitable manner known in the art, including, for example, Scatchard analysis and/or competitive binding assays, such as radioimmunoassays (RIA), enzyme immunoassays (EIA), and sandwich competition assays, and different variants thereof known in the art. In some embodiments, the affinity of the immunoglobulin or the antigen binding fragments disclosed herein are assessed using surface plasmon resonance (SPR) or flow cytometry.

In some embodiments, the immunoglobulin or the antigen binding fragments disclosed herein can be employed in any known assay method, such as competitive binding assays, direct and indirect sandwich assays, and immunoprecipitation assays for the detection and quantitation of one or more target antigens. The immunoglobulin or the antigen binding fragments will bind the one or more target antigens of PSD95 with an affinity that is appropriate for the assay method being employed. In certain embodiments, the immunoglobulin or the antigen binding fragments are used in an assay selected from the group consisting of: a radioimmunoassay, an immunohistochemistry assay, a competitive-binding assay, a Western Blot analysis, an ELISA assay, a two-dimensional gel electrophoresis, an enzyme immunoassay, a sandwich immunoassay, an immunodiffusion assay, an immunoradiometric assay, a fluorescent immunoassay, and an immunoelectrophoresis assay.

In some embodiments provided is a method of diagnosis of a neurological disorder in a subject using the immunoglobulin or the antigen binding fragments disclosed herein. The immunoglobulin or the antigen binding fragments disclosed herein can be used in immunoassays in which they can be utilized in liquid phase or bound to a solid phase carrier. In addition, the immunoglobulin or the antigen binding fragments in these immunoassays can be detectably labeled in various ways. Examples of types of immunoassays which can utilize the immunoglobulin or the antigen binding fragments disclosed herein are flow cytometry, e.g., FACS, MACS, immunohistochemistry, competitive and non-competitive immunoassays in either a direct or indirect format.

In some embodiments disclosed herein is a method for diagnosing a neurological disorder in a subject, comprising detecting in a sample from the subject PSD95 phosphorylated at threonine 19 or PSD95 phosphorylated at both threonine 19 and serine 25, wherein the sample is contacted with the immunoglobulin or the antigen binding fragments disclosed herein. In some embodiments, the sample is blood, serum, plasma, urine, feces, respiratory secretions, exosome, cerebrospinal fluid, saliva, or brain tissue. For example, a method for diagnosing a neurological disorder in a subject, comprising detecting polymerized/aggregated homomeric or heteromeric Thr19-pPSD95 containing protein complexes in a blood sample.

The immunoglobulin or the antigen binding fragments disclosed herein are also useful for imaging and diagnostic applications. The immunoglobulin or the antigen binding fragments labeled with a detectable moiety can be administered to a subject, for example into the bloodstream, and the presence and location of the labeled immunoglobulin or the antigen binding fragments in the host assayed. The immunoglobulin or the antigen binding fragments can be labeled with any moiety that is detectable in a patient, whether by nuclear magnetic resonance, radiology, or other detection means known in the art. In some embodiments, provided is an immunoconjugate comprising the immunoglobulin or the antigen binding fragments disclosed herein and a conjugating part comprising a detectable moiety. For example, the detectable moiety can be a radioisotope, such as ³H, ¹⁴C, ³²P, ³⁵S, ¹²⁵I , ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase. In some embodiments, the detectable moiety is selected from fluorophores, immuno-histochemical tracers, positron emission tomography (PET) tracers, near infrared spectrometer (NIR) probes, single-photon emission computerized tomography (SPECT), magnetic particle imaging, magnetic resonance imaging contrast agents, ultrasound contrast agents, and radio-isotopes.

In particular embodiments provided here is a nucleic acid comprising a nucleotide sequence encoding the amino acid sequence of the immunoglobulin or the antigen binding fragments, a vector comprising the nucleic acid, and a host cell comprising the expression vector.

The term “subject” is intended to include human and non-human animals, particularly mammals.

### Diagnosis of Neurological Disorders

In certain embodiments, provided herein are methods for diagnosing neurological disorders. As used herein, the term “neurological disorder” refers to any nervous system disorder that requires clinical care by a physician or other healthcare professional. Neurological disorder can also refer to any neurological and/or psychiatric disorders characterized by excessive or inadequate synapse disassembly. Examples of neurological disorders can include, but are not limited to, acute spinal cord injury, Alzheimer's disease, Amyotrophic Lateral Sclerosis (ALS), anxiety, ataxia, attention-deficit disorder, autism, behavior disorders (e.g., Attention Deficit Hyperactivity Disorder (ADHD), Behavioral Addiction, Conduct Disorder, Obsessive-compulsive disorder (OCD), and Oppositional Defiant Disorder (ODD)) Bell's palsy, brain tumors, cerebral aneurysm, cognitive decline, depression, epilepsy (and seizures), Guillain-Barré syndrome, headache, head injury, hydrocephalus, intellectual disorders (e.g., Down syndrome, fragile x syndrome, fetal alcohol syndrome, and Prader-Willi syndrome) meningitis, multiple sclerosis, muscular dystrophy, Parkinson's disease, schizophrenia, or stroke.

In certain embodiments, the immunoglobulin or the antigen binding fragments can be used in an assay, such as plate-based or other format ELISAs, for the diagnosis of a neurological disorder. In some embodiments, the assay can include a solid support, wherein one or more of the immunoglobulins or the antigen binding fragments provided herein are immobilized on the solid support. The solid support may be, but is not limited to being, the inner, and/or bottom surface of a well of a microtiter plate or a substrate that is included as part of a lateral flow device, for example. The immunoglobulins or the antigen binding fragments used in the assays described herein may be immobilized on the solid support by any methodology known in the art, including, for example, covalently or non-covalently, directly or indirectly, attaching the immunoglobulins or the antigen binding fragments to the solid support. Therefore, while these immunoglobulins or the antigen binding fragments may be attached to the solid support by physical adsorption (i.e., without the use of chemical linkers), it is also true that these immunoglobulins or the antigen binding fragments may be immobilized to the solid support by any chemical binding (i.e., with the use of chemical linkers) method readily known to one of skill in the art.

In various embodiments, an increase in PSD95 phosphorylated at threonine 19 or PSD95 phosphorylated at both threonine 19 and serine 25 includes increases in levels from 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 85%-90%, 90%-95%, 95%-100%, or by 100%, 150%, 200%, 250%, 300%, 350%, 400%, 450%, 500%, 600%, 700%, 800%, 900%, 1000%, or higher when compared to the control. This increase (e.g., 5% increase in pg/ml of PSD95 phosphorylated at threonine 19 or PSD95 phosphorylated at both threonine 19 and serine 25 in the CSF over the control) may be seen in the neurological disorder (for example, sAD), as the neurological disorder progresses or as treatment starts to fail.

In various embodiments, a decrease in PSD95 phosphorylated at threonine 19 or PSD95 phosphorylated at both threonine 19 and serine 25 includes decreases in levels from 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 85%-90%, 90%-95%, 95%-100%, or by 100%, 150%, 200%, 250%, 300%, 350%, 400%, 450%, 500%, 600%, 700%, 800%, 900%, 1000%, or lower when compared to the control. This decrease (e.g., 5% decrease in pg/ml of PSD95 phosphorylated at threonine 19 or PSD95 phosphorylated at both threonine 19 and serine 25 in the CSF over the control) may be seen as there is an improvement in the neurological disorder (e.g., from severe sAD to moderate sAD) or treatment begins to work.

In addition to diagnostic methods, the immunoglobulins and/or the antigen binding fragments thereof can be used for monitoring the progression of a neurological disorder in a patient. Such methods include obtaining a sample from the patient, detecting a level of PSD95 phosphorylated at threonine 19 or PSD95 phosphorylated at both threonine 19 and serine 25, and/or a fragment thereof. In some embodiments, a second (or third or fourth) measurement of the level of PSD95 phosphorylated at threonine 19 or PSD95 phosphorylated at both threonine 19 and serine 25, and/or fragment thereof is subsequently performed using the same steps following a time interval (e.g., at least 1, 2, 5, 7, 14, or 28 days, or at least 1, 2, 3, 4, 5, 6, 8, 10, 12, or 24 months). The two or more measurements are then compared, where an increase in levels is indicative of disease progression, while a decrease in levels is indicative of improvement.

Such monitoring methods can be performed in conjunction with administration of a therapy (e.g., pharmaceutical therapy) to the patient and, thus, can be used to determine if a particular therapy is having the desired effect on the severity of the neurological disorder. In certain embodiments, the first measurement is taken prior to commencement of a therapy. Therapy is begun following the first measurement, and a second measurement is performed (e.g., three to six months later) following the commencement of therapy. A change in the second measurement as compared to the first measurement can thus be taken as indication of the effectiveness of the therapy.

### Treatment of Neurological Disorders

As used herein, the terms “effective treatment,” “treatment,” “treat,” or “treating” refer to a method of reducing the effects of a neurological disorder or a neurological condition or symptom of the neurological disorder or neurological disease or neurological condition. Thus, in the methods disclosed herein, effective treatment can refer to a 5%, 10%, 20%, 30%, 40%, 50%, 60%, 70%, 80%, 90%, or 100% reduction in the severity of an established neurological disorder or condition or symptom of the neurological disorder or condition. For example, a method of treating a neurological disorder is considered to be an effective treatment if there is a 5% reduction in one or more symptoms of the disorder in a subject as compared to a control. Thus, the reduction can be a 5%, 10%, 20%, 30%, 40%, 50%, 60%, 70%, 80%, 90%, 100% or any percent reduction between 5% and 100% as compared to native or control levels. It is understood that treatment does not necessarily refer to a cure or complete ablation of the neurological disorder, condition, or symptoms of the neurological disorder or condition.

After a subject is found and determined to have a neurological disorder or condition, or determined to be at risk of a neurological disorder, a medical professional or a team of medical professionals will recommend one or several treatment options. In determining a treatment plan, factors to consider include the type, location, and stage of the neurological disorder, as well as the patient's overall physical health. Patients with a neurological disorder are typically managed by a health care team made up of doctors from different specialties, such as: a neurologist, a neuropsychologist, a geriatrician, a geriatric psychiatrist, a geropsychologist, and/or a physical therapist. After a subject is found and determined to have a neurological disorder or condition, or determined to be at risk of a neurological disorder, a medical professional or a team of medical professionals will typically recommend one or several treatment options including one or more of small molecule medications (Anticholinergics, Antidepressants, antipsychotics, anti-inflammatory/immunosuppressant agents), therapeutic monoclonal antibodies targeting pathological proteins (for example, Aducanumab, Bapineuzumab, humanized 3D6; Solanezumab, humanized m266; Gantenerumab; Crenezumab; BAN2401, humanized mAb158; GSK 933776; AAB-003 (Fc-engineered bapineuzumab); SAR228810, humanized 13C3; or BIIB037/BART), chelation agents, Glycogen synthase kinase 3 (GSK-3) inhibitors (e.g., lithium, lithium salts, naproxen, and/or curcumin), neurogenic and synapto-genic factors (growth factors), neuronal stem cells, gene therapies (ApoE alleles), nutrient supplements (DHA, choline, riboflavin, antioxidants), non-pharmacological approaches such cognitive training and trans-cranial electrical stimulation.

### Assays and Kits

In certain embodiments, this disclosure provide a diagnostic kit or assay useful for detecting PSD95 phosphorylated at threonine 19 in a neurological disorder. In certain embodiments, this disclosure provide a diagnostic kit or assay useful for detecting PSD95 phosphorylated at serine 25 in a neurological disorder. In certain embodiments, this disclosure provide a diagnostic kit or assay useful for detecting PSD95 phosphorylated at both threonine 19 and serine 25 in a neurological disorder. In some embodiments, the kit or assay comprises an immunoglobulin and/or the antigen binding fragment capable of binding specifically to PSD95 phosphorylated at threonine 19, at serine 25, and/or at both threonine 19 and serine 25 and a labeled antibody being capable of binding specifically to the immunoglobulin and/or the antigen binding fragment.

In some embodiments, a diagnostic kit or assay useful for detecting PSD95 phosphorylated at threonine 19 is provided. In some embodiments, a diagnostic kit or assay useful for detecting PSD95 phosphorylated at serine 25 is provided. In some embodiments, a diagnostic kit or assay useful for detecting PSD95 phosphorylated at both threonine 19 and serine 25 is provided. For example, an ELISA assay that utilizes the immunoglobulin and/or the antigen binding fragment disclosed herein as a capture antibody that specifically binds to PSD95 phosphorylated at threonine 19. This antibody can be combined with a reporter antibody, for example, which can bind to total PSD95. This combination of reporter antibody and capture antibody in one assay can be utilized alone or combined with other biomarkers for diagnosing and/or monitoring the progression or regression of a neurological disorder.

In certain embodiments, any of a variety of known immunoassay methods can be used for detection and quantification of PSD95 phosphorylated at threonine 19 and/or PSD95 phosphorylated at both threonine 19 and serine 25, including, but not limited to, immunoassay, using an antibody specific for the PSD95 phosphorylated at threonine 19 and/or PSD95 phosphorylated at both threonine 19 and serine 25, e.g., by enzyme-linked immunosorbent assay (ELISA), radioimmunoassay (RIA), and the like; and functional assays for the PSD95 phosphorylated at threonine 19 and/or PSD95 phosphorylated at both threonine 19 and serine 25, e.g., binding activity or enzymatic activity.

To increase the sensitivity of the assay, an immunocomplex (i.e., bound antibody and sample) may be further exposed to a second antibody (e.g., a reporter antibody), which is labeled and binds to the first antibody or to the PSD95. Typically, the secondary antibody comprises a detectably moiety, e.g., with a fluorescent marker so it can be easily visualized by any method (e.g., by eye, microscope, or machine). As used herein, the terms “label” or “labeled” refers to incorporation of a detectable moiety on the protein, peptide, antibody, or fragment thereof, e.g., radiolabeled moiety, fluorescent label (e.g., FITC, rhodamine, lanthanide phosphors, dye quencher pairs, etc.), enzymatic label (e.g., horseradish peroxidase, beta-galactosidase, luciferase, alkaline phosphatase), chemiluminescent labels mass tags, histidine tags, chemiluminescent labels, or biotinyl moieties that can be detected by marked avidin (e.g., streptavidin or neutravidin containing a fluorescent marker or enzymatic activity that can be detected by optical or calorimetric methods).

Generally a sandwich ELISA or modified ELISA methods comprise contacting the sample with the immunoglobulins and/or the antigen binding fragments thereof specific to PSD95 phosphorylated at threonine 19 or PSD95 phosphorylated at both threonine 19 and serine 25. The immunoglobulins and/or the antigen binding fragments thereof utilized may be a monoclonal antibody, immobilized to a support or it may be a polyclonal antibody that binds to more than one epitope. After allowing the sample time to bind with the antibody and washing of unbound sample, a labeled antibody is contacted with the sample or, in various embodiments, the capture antibody and sufficient time is allowed for the labeled antibody to specifically bind to PSD95 phosphorylated at threonine 19 or PSD95 phosphorylated at both threonine 19 and serine 25 or the capture antibody. The bound label is detected and thus the PSD95 is detected and can be quantified.

In some embodiments, total PSD95 levels and/or non-modified PSD95 (non-phosphorylated PSD95) can also be measured, and the measured levels can be expressed as a ratio of Thr19-pPSD95 to total PSD95.

The assays and methods can be provided as part of a kit. Thus, in various embodiments, diagnostic kits for detecting the presence/absence and/or a level of expression of PSD95 phosphorylated at threonine 19 and/or PSD95 phosphorylated at both threonine 19 and serine 25 in a sample are provided. The kits may contain one or more antibodies (e.g., monoclonal antibodies, polyclonal antibodies, labeled and unlabeled), fragments thereof for detecting PSD95 phosphorylated at threonine 19 and/or PSD95 phosphorylated at both threonine 19 and serine 25. The kit may optionally provide additional components that are useful in the procedure, including, but not limited to, buffers, developing reagents, labels, reacting surfaces, means for detection, control samples, standards, instructions, and interpretive information.

The reference level used for comparison with the measured level for PSD95 phosphorylated at threonine 19 and/or PSD95 phosphorylated at both threonine 19 and serine 25 may vary, depending on the embodiment employed. As used herein, a “reference level” may be a predetermined reference level, such as an average of levels obtained from a population that is not afflicted with the neurological disorder, but in some instances, the reference level can be a mean or median level from a group of individuals including patients with said neurological disorder. In some instances, the predetermined reference level is derived from (e.g., is the mean or median of) levels obtained from an age-matched population.

### Vectors and Host Cells

In some aspects, this disclosure provides isolated nucleic acid sequences encoding an immunoglobulin or antigen binding fragment thereof as disclosed herein. For example, vector constructs comprising a nucleotide sequence encoding the immunoglobulin or antigen binding fragments thereof that specifically binds PSD95 phosphorylated at threonine 19, at serine 25, and/or both threonine 19 and serine 25, as disclosed herein, host cells comprising such a vector, and recombinant techniques for the production of the immunoglobulin or antigen binding fragments thereof.

The terms “recombinant” and “engineered,” as used herein and applied to a particular molecule, such as a polypeptide, refers to a molecule that has been modified or manipulated, such as by mutation, truncation, deletion, substitution, addition, conjugation, or by otherwise changing the primary sequence, chemical or three-dimensional structure, chemical signature, folding behavior, glycosylation state, or any other attribute of the molecule, such that the molecule differs from its naturally occurring counterpart.

The term “vector,” as used herein, refers to any molecule (e.g., nucleic acid, plasmid, or virus) that is used to transfer coding information to a host cell. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “expression vectors.”

Vectors normally contain components known in the art and generally include, but are not limited to, one or more of the following: a signal sequence, an origin of replication, one or more marker or selection genes, sequences facilitating and/or enhancing translation, an enhancer element and so on. Thus, expression vectors include a nucleotide sequence operably linked to such suitable transcriptional or translational regulatory nucleotide sequences such as those derived from mammalian, microbial, viral, or insect genes. Examples of additional regulatory sequences include operators, mRNA ribosomal binding sites, and/or other appropriate sequences which control transcription and translation, such as initiation and termination thereof. Nucleotide sequences are “operably linked” when the regulatory sequence functionally relates to the nucleotide sequence for the appropriate polypeptide. Thus, a promoter nucleotide sequence is operably linked to, e.g., the antibody heavy chain sequence if the promoter nucleotide sequence controls the transcription of that nucleotide sequence.

The vector may be a plasmid, a single-stranded or double-stranded viral vector, a single-stranded or double-stranded RNA or DNA phage vector, a phagemid, a cosmid or any other carrier of a transgene of interest. Such vectors may be introduced into cells as polynucleotides by well-known techniques for introducing DNA and RNA into cells. The vectors, in the case of phage and viral vectors also may be introduced into cells as packaged or encapsulated virus by well-known techniques for infection and transduction. Viral vectors may be replication competent or replication defective.

The immunoglobulin or antigen binding fragments thereof that specifically binds PSD95 phosphorylated at threonine 19 or PSD95 phosphorylated at both threonine 19 and serine 25 as disclosed herein can be expressed from any suitable host cell. Examples of useful host cells include prokaryotic, yeast or higher eukaryotic cells and include but are not limited to microorganisms such as bacteria transformed with recombinant bacteriophage DNA, plasmid DNA or cosmid DNA expression vectors containing the antibody coding sequences of interest; yeast (e.g., Saccharomyces, Pichia, Actinomycetes, Kluyveromyces, Schizosaccharomyces, Candida, Trichoderma, Neurospora, and filamentous fungi, such as Neurospora, Penicillium, Tolypocladiumand Aspergillus) transformed with recombinant yeast expression vectors containing antibody coding sequences; insect cell systems infected with recombinant virus expression vectors (e.g., Baculovirus) containing antibody coding sequences; plant cell systems infected with recombinant virus expression vectors (e.g., cauliflower mosaic virus, CaMV; or tobacco mosaic virus, TMV) or transformed with recombinant plasmid expression vectors (e.g., Ti plasmid) containing antibody coding sequences; or mammalian cell systems (e.g., COS, CHO, BHK, 293 or cells) harboring recombinant expression constructs containing promoters derived from the genome of a mammalian.

The terms “cell,” “cell line,” and “cell culture” include progeny thereof. It is also understood that all progeny may not be precisely identical, such as in DNA content, due to deliberate or inadvertent mutation. Variant progeny that have the same function or biological property of interest, as screened for in the original cell, are included. The “host cells” used in the present disclosure generally are prokaryotic or eukaryotic hosts, selected as a design choice.

“Transformation” of a cellular organism, cell or cell line with a nucleic acid means introducing a nucleic acid into the target cell so that the nucleic acid is replicable, either as an extrachromosomal element or by chromosomal integration, and, optionally, expressed. “Transfection” of a cell or organism with a nucleic acid refers to the taking up of the nucleic acid, e.g., an expression vector, by the cell or organism whether or not any coding sequences are in fact expressed. The terms “transfected host cell” and “transformed” refer to a cell in which a nucleic acid was introduced. Typical prokaryotic host cells include various strains of E. coli. Typical eukaryotic host cells are mammal cells, such as Chinese hamster ovary, or cells of human origin. The introduced nucleic acid sequence may be from the same species as the host cell or of a different species from the host cell, or may be a hybrid nucleic acid sequence, containing some foreign and some homologous nucleic acids.

The term “operably linked,” as used herein, refers to an arrangement of flanking sequences wherein the flanking sequences so described are configured or assembled so as to perform their usual function. Thus, a flanking sequence operably linked to a coding sequence may be capable of effecting the replication, transcription, and/or translation of the coding sequence. For example, a coding sequence is operably linked to a promoter when the promoter is capable of directing transcription of that coding sequence. A flanking sequence need not be contiguous with the coding sequence, so long as it functions correctly.

The term “host cell,” as used herein, refers to a cell into which an expression vector has been introduced. A host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the amino acid sequences, proteins, or recombinant polypeptides of the disclosure, including bacterial, yeast, baculoviral, insect and mammalian expression systems (as well as phage display expression systems).

In some embodiments, a host cell is chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the expressed antibody of interest. Hence, eukaryotic host cells which possess the cellular machinery for proper processing of the primary transcript, glycosylation and phosphorylation of the gene product may be used. Such mammalian host cells include, but are not limited to, CHO, COS, 293, 3T3 or myeloma cells.

For long-term, high-yield production of recombinant proteins, stable expression is preferred. For example, cell lines which stably express the immunoglobulin or antigen binding fragments as disclosed herein may be engineered. Rather than using expression vectors which contain viral origins of replication, host cells can be transformed with DNA controlled by appropriate expression control elements (e.g., promoter, enhancer, sequences, transcription terminators, polyadenylation sites etc.) and a selectable marker. Following the introduction of the foreign DNA, engineered cells may be allowed to grow for one to two days in an enriched media, and then are moved to a selective media. The selectable marker in the recombinant plasmid confers resistance to the selection and allows cells to stably integrate the plasmid into a chromosome and be expanded into a cell line. Such engineered cell lines not only are useful for antibody production but are useful in screening and evaluation of compounds that interact directly or indirectly with the antibody molecule.

In some embodiments, the disclosure provides methods for preparing the immunoglobulin or antigen binding fragments thereof that specifically binds PSD95 phosphorylated at threonine 19 or PSD95 phosphorylated at both threonine 19 and serine 25 as disclosed herein, which methods comprise cultivating or maintaining a host cell under conditions such that the host cell produces or expresses the amino acid sequence, protein, or recombinant polypeptide, and optionally further comprises isolating the amino acid sequence, protein, or recombinant polypeptide so produced.

Without limiting the disclosure, a number of embodiments of the disclosure are described below for purpose of illustration.

The disclosure will be further described in the following examples, which do not limit the scope of the disclosure described in the claims.

### EXAMPLES

The Examples that follow are illustrative of specific embodiments of the disclosure, and various uses thereof. They are set forth for explanatory purposes only, and should not be construed as limiting the scope of the disclosure in any way.

### Materials and Methods

Biochemical experiments: Detailed methods used for antigen conjugate preparation, peptide adduction and pH-dependent reversibility experiments, peptide crosslinking experiments, and protein crosslinking experiments are provided in the materials and methods below (also see Ramsden et al., J Alzheimers Dis. 2022; 87(3):1251-1290, which is incorporated by reference herein in its entirety, for antibody sources and technical specifications). Snakeskin dialysis tubing and Slide-A-lyzer dialysis units were obtained from Thermo Fisher. Spectra/Por dialysis apparatus and tubing were obtained from Repligen (Waltham, MA). Dialyses were performed using appropriate MWCO for protein or peptide molecular weights against 3 changes of the buffers specified below. KPhos refers to potassium phosphate buffer, pH 7.4 (Sigma, St. Louis, MO). 10 mM PBS refers to 1x PBS, pH 7.4 (Quality Biological, Gaithersburg, MD).

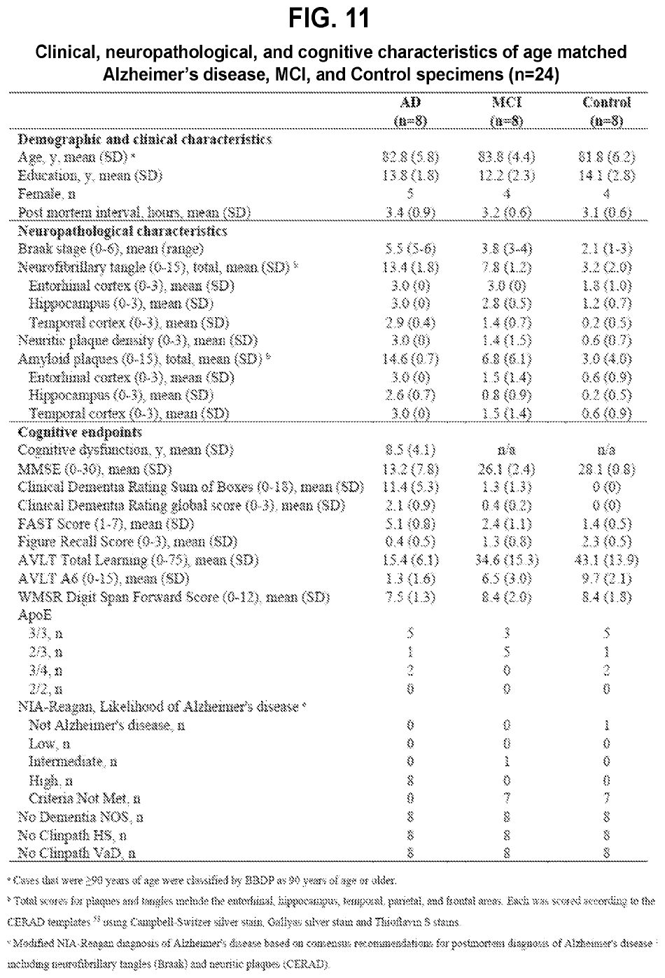

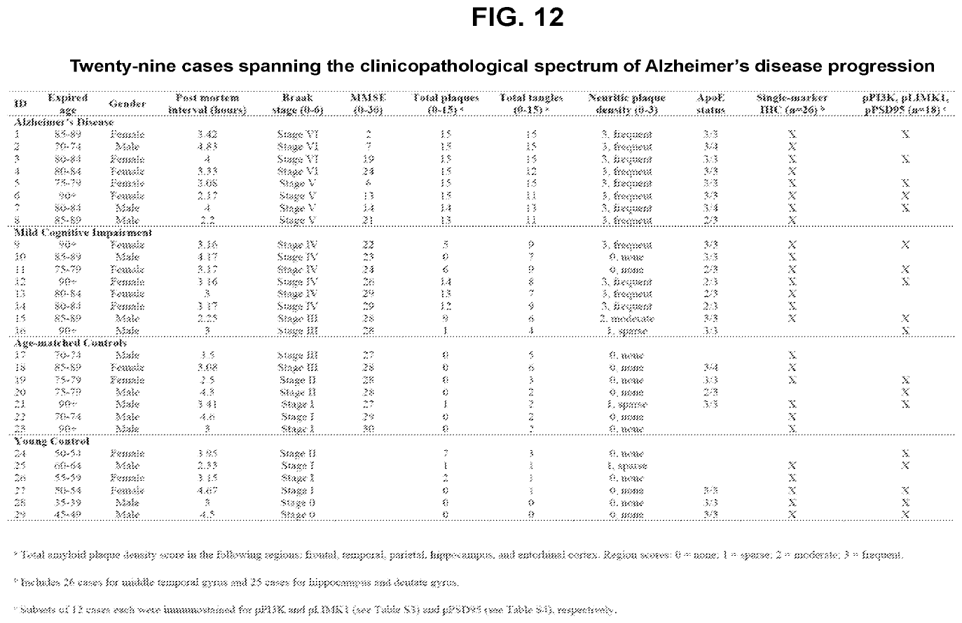

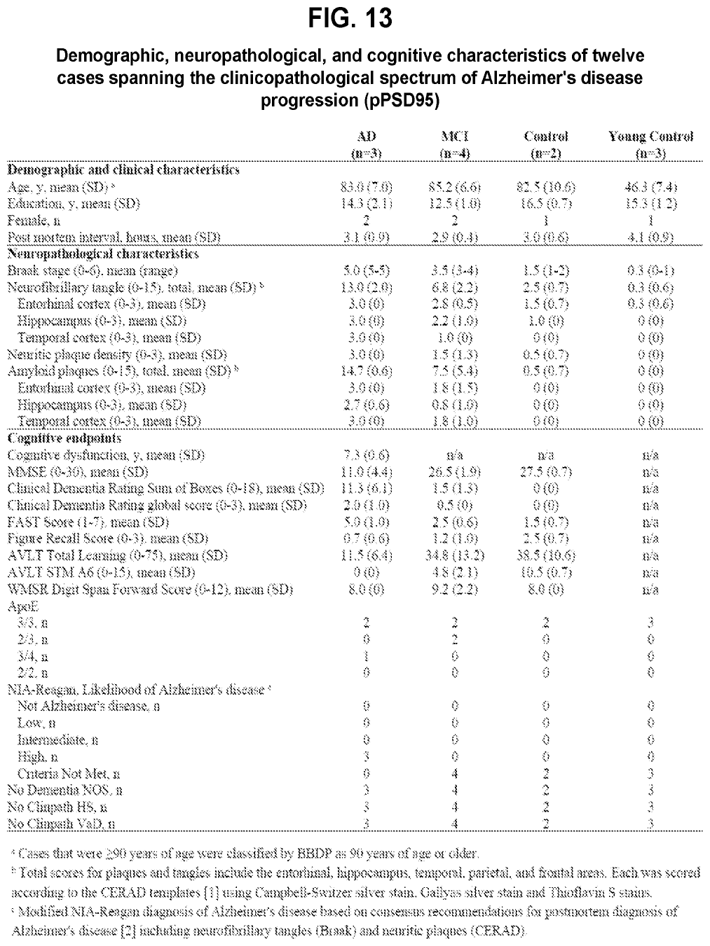

Postmortem specimens: 6 μm-thick coronal medial temporal lobe paraffin sections at the level of the body of the hippocampus were obtained from a total of 29 cases spanning the clinicopathological spectrum of sAD, from the Brain and Body Donation Program (BBDP) at the Banner Sun Health Research Institute (FIGS. 11-13). To mitigate limitations due to tissue degradation, BBDP employed a rapid on-call autopsy team to achieve short PMI (mean 3 hours). Standardized fixation procedures were employed with 1 cm sections fixed in 10% formalin for 48 hours. All BBDP subjects provided written consent for study procedures, autopsy and sharing of de-identified data prior to enrollment. The study and its consenting procedures were approved by the Western Institutional Review Board of Puyallup, Washington. The Banner BBDP population has been extensively described by Beach et al. Briefly, most BBDP donors were enrolled as cognitively normal volunteers residing in the retirement communities near Phoenix, Arizona, USA. Following provision of informed consent, donors received standardized medical, neurological, and neuropsychological assessments during life. Neuropathological and cognitive examinations and endpoints captured by BBDP are detailed in a previous publication. Antemortem Mini-Mental Status Exam (MMSE) test had the least missing data and was the main cognitive endpoint of for the present study.

Neuropathological procedures and examinations performed by BBDP has been previously published. Briefly, the Braak neurofibrillary stage (0-VI) was determined using thick 40- 80-micron sections stained with Gallyas, Campbell-Switzer and thioflavine S stains as originally defined by Braak and Braak. Senile plaque density—including neuritic, cored, and diffuse plaques—was assessed in standard regions of the frontal, temporal, and parietal lobes, hippocampal CA1 region and entorhinal/transentorhinal region. Each region was assigned a semi-quantitative score of none, sparse, moderate and frequent and converted to numerical values 0-3, according to the CERAD templates. Plaque total is the arithmetic sum of scores from these five regions ranged from 0-15. Neurofibrillary tangle density was assessed in the same five regions, with CERAD templates used to obtain semi-quantitative scores of none, sparse, moderate and frequent and these are converted to numerical values 0-3. Tangle total is the arithmetic sum of scores from these five regions ranged from 0-15. The NIA-Reagan consensus recommendations were used for postmortem diagnosis of AD with high, intermediate and low referring to the likelihood that dementia, if present, is due to AD histopathology. AD was at a minimum defined as intermediate or high NIA-Reagan criteria. Mild Cognitive Impairment denoted the presence of this diagnosis at the time of death. A control designation is a participant without dementia or parkinsonism during life and without a major neuropathological diagnosis.

Single-marker immunohistochemistry (IHC): Single-marker IHC was performed by Histoserv (Gaithersburg, MD, USA). Combined blocks containing temporal and/or frontal cortex from sAD cases and non-AD controls were sectioned into 6 μm-thick coronal sections. Optimal immunostaining conditions were then empirically determined using an incremental heat induced epitope retrieval (HIER) method using 10 mM Na/Citrate pH6 and/or TRIS/EDTA pH9 buffer under following heating conditions: 70° C./10 min, 70° C./20 min, 70° C./40 min. Selected antibodies with suboptimal HIER treatment of up to 40 minutes underwent additional retrieval rounds using formic acid (88%, 20 min) or proteinase K (Dako S3020) for 5-10 min at room temperature (RT). Secondary antibodies used for single IHC chromogenic staining were appropriately matched to the host class/subclass of the primary antibody. These combined sAD plus Control blocks were used to generate negative controls (comparing staining results versus pre-immune serum and with the primary antibody omitted) and positive controls (comparing staining results in temporal or frontal cortex specimens from confirmed AD cases with known region-specific Aβ and tangle scores versus non-AD controls). These empirically determined optimal conditions were then used to immunolabel sets of entorhinal-hippocampal slides using IHC and MP-IHC (as described below). Briefly, for IHC, sections were first deparaffinized using standard Xylene/Ethanol/Rehydration protocol followed by antigen unmasking with 10-40 min HIER step in 10 mM Tris/EDTA buffer pH9 (Tris/EDTA buffer) as described above (see Ramsden et al., J Alzheimers Dis. 2022; 87(3):1251-1290, which is incorporated by reference herein in its entirety, for antibody sources and technical specifications).

Multiplex Fluorescence Immunohistochemistry and In Situ Hybridization: Multiplex fluorescence immunohistochemistry (MP-IHC) and multiplex fluorescence in situ hybridization (MP-ISH) were performed on 6 μm-thick coronal sections that were collected on Leica Apex Superior adhesive slides (VWR, 10015-146) to prevent tissue loss. We completed up to 6 iterative rounds of sequential immunostaining with select antibody panels targeting the ApoE/Reelin-ApoE receptor-Dab1 axis and classical biomarkers of AD pathology, alongside classical cytoarchitectural biomarkers to map brain tissue parenchyma (see Ramsden et al., J Alzheimers Dis. 2022; 87(3):1251-1290, which is incorporated by reference herein in its entirety). Fluorophore conjugated targets from each round were imaged by multispectral epifluorescence microscopy followed by antibody stripping and tissue re-staining steps to repeat the cycle, as previously described (Maric et al. (2021) “Wholebrain tissue mapping toolkit using large-scale highly multiplexed immunofluorescence imaging and deep neural networks. Nat Commun 12, 1550), each time using a different antibody panel. MP-ISH was carried out on a subset of sections to screen for ApoER2 mRNA using a custom designed RNA probe and standard RNAscope Multiplex Fluorescent V2 hybridization kits (Advanced Cell Diagnostics, Inc.) per manufacturer's instructions. Sections probed with RNAscope were subsequently processed with 2 sequential rounds of MP-IHC to facilitate RNAprotein co-detection. Briefly, for screening tissues by MP-IHC and/or MP-ISH, the sections were first deparaffinized using standard Xylene/Ethanol/Rehydration protocol followed by HIER antigen unmasking step in Tris/EDTA buffer for 10-15 min using an 800 W microwave set at 100% power. Sections intended for MPISH were then processed using the RNAscope V2 kit (Advanced Cell Diagnostics, Inc.). All sections to be sequentially processed for iterative MP-IHC screening were first incubated with Human BD Fc Blocking solution (BD Biosciences) to saturate endogenous Fc receptors, and then in True Black Reagent (Biotium) to quench intrinsic tissue autofluorescence. Sections were then immunoreacted for 1 hour at RT using cocktail mixture of immune-compatible antibody panels targeting ApoE/Reelin-ApoE receptor-Dab1 signaling cascade pathways, classical biomarkers of AD pathology, and brain cytoarchitectural biomarkers. This step was followed by washing off excess primary antibodies in PBS supplemented with 1 mg/ml bovine serum albumin (BSA) and staining the sections using a 1 μg/ml cocktail mixture of the appropriately cross-adsorbed secondary antibodies (purchased from either Thermo Fisher, Jackson ImmunoResearch or Li-Cor Biosciences) conjugated to one of the following spectrally compatible fluorophores: Alexa Fluor 430, Alexa Fluor 488, Alexa Fluor 546, Alexa Fluor 594, Alexa Fluor for use under a CCO license. 647, IRDye 600LT, or IRDye 800CW. After washing off excess secondary antibodies, sections were counterstained using 1 μg/ml DAPI (Thermo Fisher Scientific) for visualization of cell nuclei. Slides were then coverslipped using Immu-Mount medium (Thermo Fisher Scientific) and imaged using a multispectral epifluorescence microscope (see below). After imaging, tissue bound primary and secondary antibodies were both stripped off the slides after a 5-minute incubation at RT in NewBlot Nitro 5X Stripping buffer (Li-Cor Biosciences) followed by 1-minute additional HIER step in Tris/EDTA buffer. The above processing cycle beginning with re-blocking of tissues in Human BD Fc Blocking solution was repeated and the same sections then incubated using an additional panel of antibodies of interest.

Multiplex Fluorescence Immunohistochemistry Image Acquisition and Computational Reconstruction: Images were acquired from MP-IHC and/or MP-ISH probed specimen sections using the Axio Imager Z2 slide scanning fluorescence microscope (Zeiss) equipped with a 20X/0.8 Plan-Apochromat (Phase-2) non-immersion objective (Zeiss), a high resolution ORCA-Flash4.0 sCMOS digital camera (Hamamatsu), a 200 W X-Cite 200DC broad band lamp source (Excelitas Technologies), and 8 customized filter sets (Semrock) optimized to detect the following fluorophores: DAPI, Alexa Fluor 430, Alexa Fluor 488 (or Opal 520), Alexa Fluor 546 (or Opal 570), Alexa Fluor 594 (or Opal 620), Alexa Fluor 647, IRDye 680LT (or Opal 690) and IRDye 800CW. Image tiles (600×600 μm viewing area) were individually captured at 0.325 micron/pixel spatial resolution, and the tiles seamlessly stitched into whole specimen images using the ZEN 2 image acquisition and analysis software program (Zeiss), with an appropriate color table having been applied to each image channel to either match its emission spectrum or to set a distinguishing color balance. Pseudo-colored stitched images acquired from 1 round of MP-ISH and/or up to 6 rounds of MP-IHC staining and imaging were then exported to Adobe Photoshop and overlaid as individual layers to create multicolored merged composites. Multi-channel images acquired from multi-round IHC staining and repeatedly imaged tissue samples were computationally registered at the subpixel level using affine transformation, and the aligned fluorescence images corrected for photobleaching, autofluorescence, background signals, non-uniform illumination, and spectral bleed-through artifacts, as previously described by Maric et al. (2021) Whole-brain tissue mapping toolkit using large-scale highly multiplexed immunofluorescence imaging and deep neural networks. Nat Commun 12, 1550. [PubMed: 33692351].

Regional Annotation and Quantitation: IHC images were uploaded into HALO 3.1 image analysis software (Indica Labs, Corrales, NM). Boundaries of each anatomical region of interest (hippocampus, molecular layer of the dentate gyrus, gray matter of middle temporal gyrus) were identified and annotated using a combination of anatomical landmarks (lateral ventricle, fimbria, fimbria-dentate and hippocampal fissures, collateral sulcus, dentate granule

cells, cornu ammonis) and cytoarchitectonic mapping as described by Amunts et al. [78] MP-IHC, which enabled immunolabeling of numerous cytoarchitectural markers in each section, assisted in the identification of anatomical landmarks such as the dentate granule cell layer and boundaries including white matter tracts and the gray matter-white matter interfaces. Stain positive area as a percentage of each annotated region was quantified using the HALO Area Quantification v2.2.1 module. Plaque-associated objects per mm2 within each annotated region were identified and quantified using the HALO Object Colocalization v1.3 module with classifier function enabled.

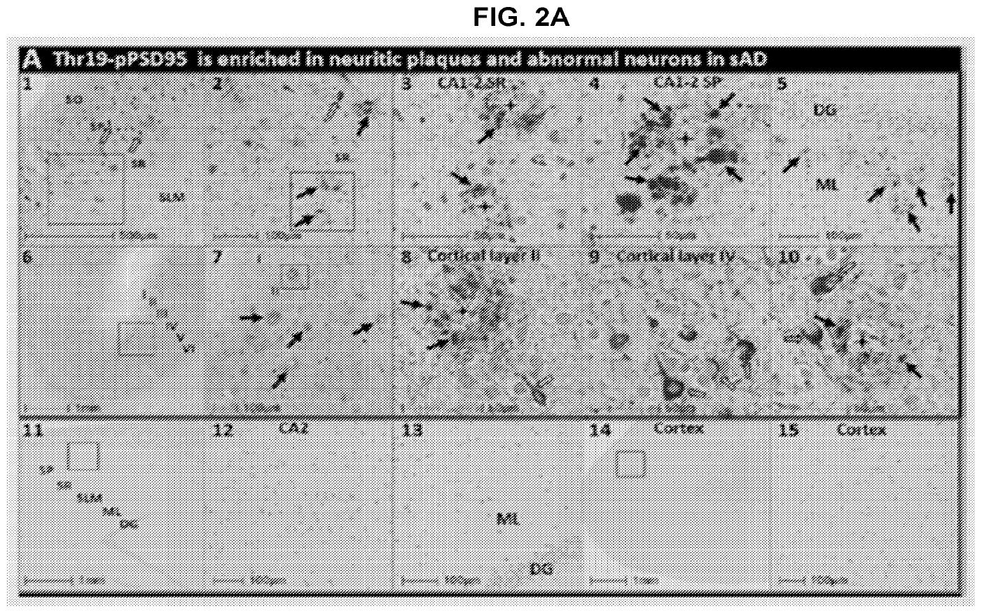

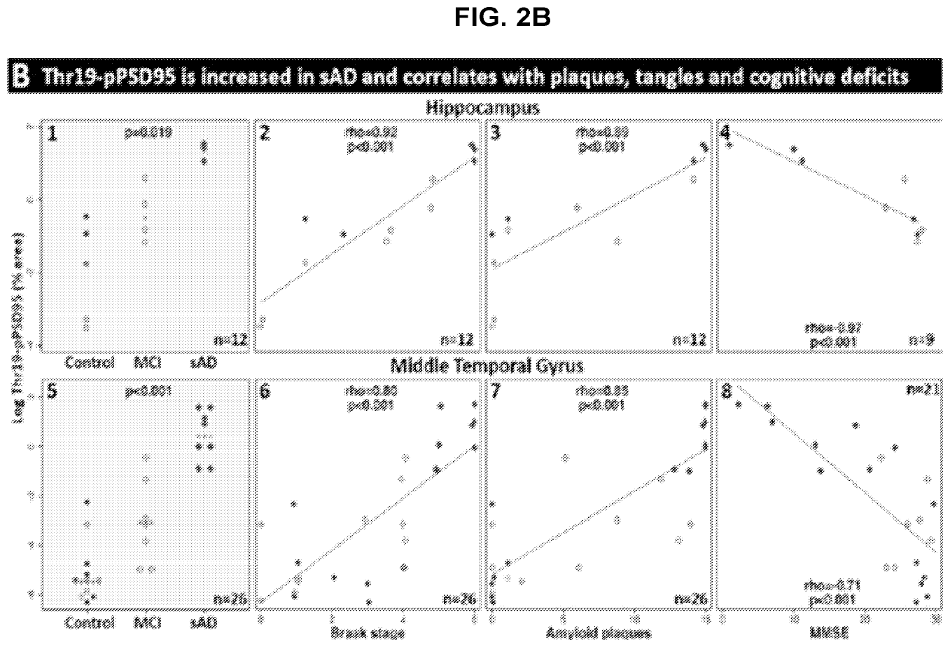

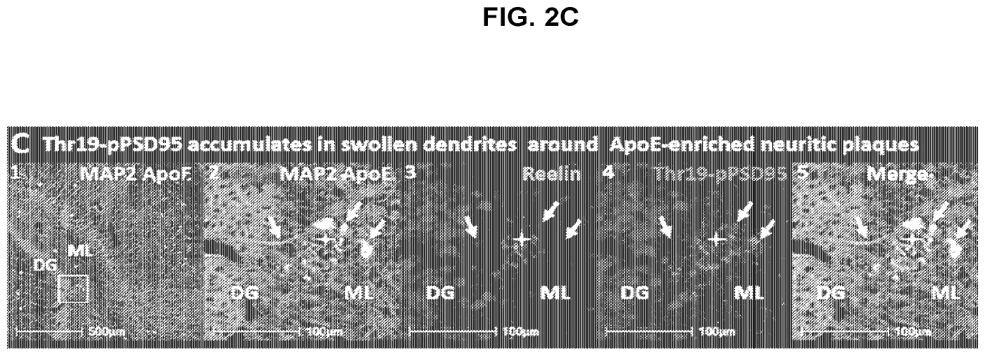

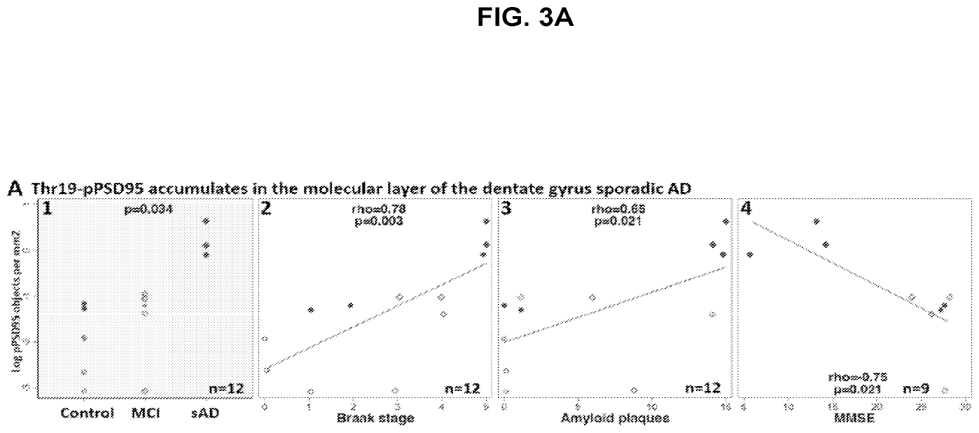

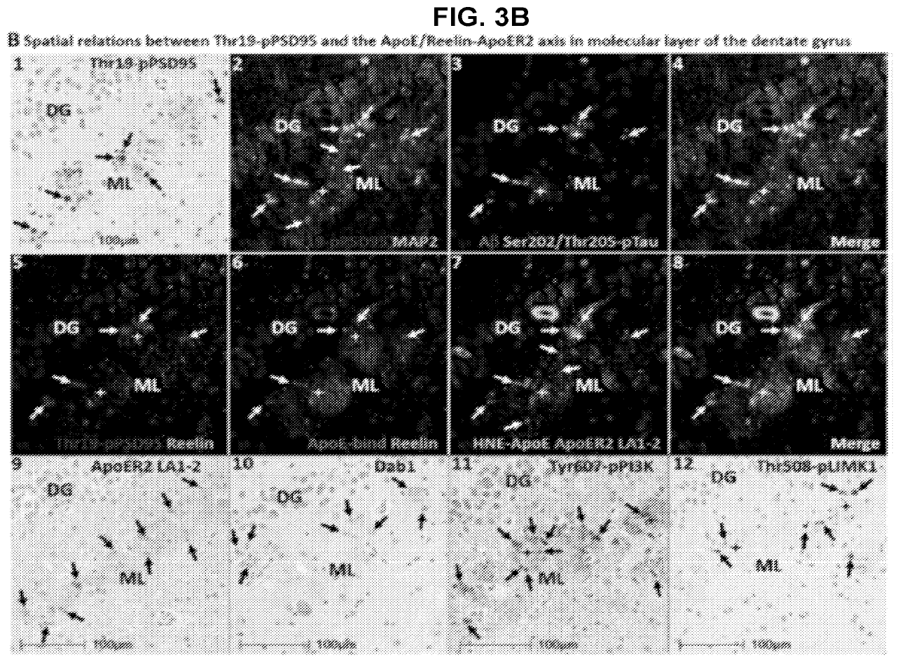

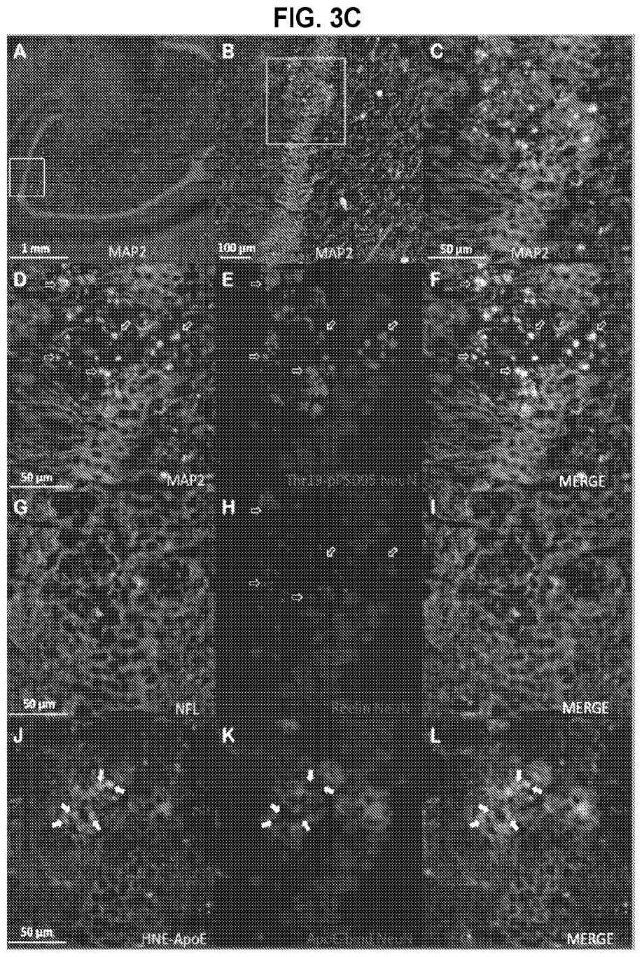

Statistical analysis: For each annotated region, differences between sAD, MCI, and Control groups according to each marker were quantified using Kruskal-Wallis tests. Variable transformations (e.g., natural log) were used as necessary. A Spearman's correlation coefficient between each immunohistochemical marker and each AD endpoint (Braak score, Total Aβ plaque load, MMSE) was calculated. Statistical analyses were conducted in Stata Release 16 (College Station, TX). No adjustments were made for multiple comparisons. Four-panel graphs showing individual data points in each group and their relationships to AD endpoints are provided in FIGS. 2B and 3A.

Neuropathological Assessments: The neuropathological assessments and endpoints captured by BBDP are detailed in Beach et al., (2015) Arizona Study of Aging and Neurodegenerative Disorders and Brain and Body Donation Program. Neuropathology 35, 354-389. Briefly, the Braak neurofibrillary stage (0-VI) was determined using thick 40-80-micron sections stained with Gallyas, Campbell-Switzer and thioflavine S stains as originally defined by Braak and Braak. Senile plaque density—including neuritic, cored, and diffuse plaques—was assessed in standard regions of the frontal, temporal, and parietal lobes, hippocampal CA1 region and entorhinal/transentorhinal region. Each region was assigned a semi-quantitative score of none, sparse, moderate and frequent and converted to numerical values 0-3, according to the CERAD templates. Plaque total is the arithmetic sum of scores from these five regions ranged from 0-15. Neurofibrillary tangle density was assessed in the same five regions, with CERAD templates used to obtain semi-quantitative scores of none, sparse, moderate and frequent and these are converted to numerical values 0-3. Tangle total is the arithmetic sum of scores from these five regions ranged from 0-15. The NIA-Reagan consensus recommendations were used for postmortem diagnosis of AD with high, intermediate and low referring to the likelihood that dementia, if present, is due to AD histopathology. AD was at a minimum defined as intermediate or high NIA-Reagan criteria. Mild Cognitive Impairment denoted the presence of this diagnosis at the time of death. A control designation is a participant without dementia or parkinsonism during life and without a major neuropathological diagnosis.

Braak Stage: describing topographical progression of neurofibrillary tangles, dystrophic neurites and neuropil threads, throughout transentorhinal and entorhinal areas, CA1 subfield of hippocampus, amygdala and cerebral neocortex. Evaluations were made, similarly as the original publication, in large (3 cm×5 cm) thick (40 or 80 μm) sections stained with the Campbell-Switzer silver stain, Gallyas silver stain and Thioflavin S stains. Final judgment of tangle density is made on the basis of combined impression from all three stains. For three years, all cases were also stained with the AT8 antibody for phosphorylated tau protein. Note that the AT8 stain has been reported to give higher Braak stages as more neurites are apparent.

Tangle Total: Average neurofibrillary tangle density in the cortex of the frontal lobe, including superior, middle, and inferior frontal gyri; cortex of the temporal lobe; cortex of the parietal lobe; CA1 subfield of hippocampus; and entorhinal cortex. Tangle density scored according to the CERAD templates, as described for the Braak stage above.

Plaque Total: Average senile (amyloid) plaque density (all types of plaques considered together) in the cortex of frontal lobe, including superior, middle, and inferior frontal gyri; cortex of temporal lobe; cortex of parietal lobe; CA1 subfield of hippocampus; and entorhinal cortex. Plaque density scored according to CERAD templates, using large (3 cm×5 cm) thick (40 or 80 μm) sections stained with Campbell-Switzer and Gallyas silver stains, and Thioflavin S stains. Validity and accuracy of this combination for estimating density of Aβ deposits established in BBDP laboratories through strong correlations with autoradiographic binding of Florbetapir (amyloid imaging ligand), with biochemical measures (ELISA) of Aβ in human cerebral cortex extracts and with quantitative measures (percentage of section area occupied) of an immunohistochemical stain for Aβ.

Neuritic Plaque Density: Greatest neuritic plaque density observed across frontal, temporal and parietal cortex regions, scored according to CERAD templates. Evaluations made in large (3 cm×5 cm) thick (40 or 80 μm) sections stained with the Campbell-Switzer silver stain, Gallyas silver stain and Thioflavin S stains. Final judgment of plaque density made on the basis of the combined impression from all three stains.

### Cognitive Assessments

The cognitive examinations and endpoints captured by BBDP are summarized below.

MMSE Test Score: Folstein Mini Mental State Examination score (0-30) obtained most proximal to death; includes MMSE scores obtained through BBDP research clinical visits and by review of private medical records.

CDR Sum: Sum of Boxes (subsection) of the Clinical Dementia Rating (CDR) Scale. CDR is widely used for staging dementia severity.

FAST Score: FAST is a functional assessment based both on caregiver report and clinician's observations: 1=normal; 2=subjective (only) report of forgetfulness or work difficulties; 3=observed early executive dysfunction; 4=definite memory and/or executive dysfunction; 5=some decline in basic activities of daily living (ADLs); 6 (a-e) definite decline in basic ADLs, with the final stage (e) being fecal incontinence; 7 (a-e) progressive loss of speech and motor abilities, with the final stage (e) being loss of the ability to hold up the head independently.

Figure Recall Score: Subject is asked to copy three simple figures, and after delay, is asked to reproduce the figures from memory. The score is the number correctly reproduced.

Rey Auditory Verbal Learning Test (AVLT): evaluates short-term auditory-verbal memory, rate of learning, learning strategies, retroactive, and proactive interference, presence of confabulation, of confusion in memory processes, retention of information, and differences between learning and retrieval. Participants are given list of 15 unrelated words repeated over five trials and asked to repeat as many words as possible. After the five trials, the number of recalled words is summed as “AVLT Total Learning Score” (0-75 scale). After the fifth learning trial, another list of 15 unrelated words is provided. The participant recalls as many words as possible from this distracter list. After a brief delay, the participant is asked to repeat the original list of 15 words (AVLT A6).

### Example 1: Thr19-Phosphorylated PSD95 is Higher in sAD Cases than Controls and Positively Correlates with Histological Progression and Cognitive Deficits

Sporadic Alzheimer's disease (SAD), which accounts for >95% of AD cases, lacks effective, disease modifying treatments. While the etiology of familial AD—including the genetic source of increased amyloid beta (Aβ) synthesis—is well-understood, the mechanisms underlying sAD are more complex and the initiating molecular lesions are unknown. The early stages of sAD are pathologically characterized by degeneration of the neural circuitry that underlies memory formation. Identification of the molecular determinants underlying this selective anatomical vulnerability may provide clues to the origins of sAD.

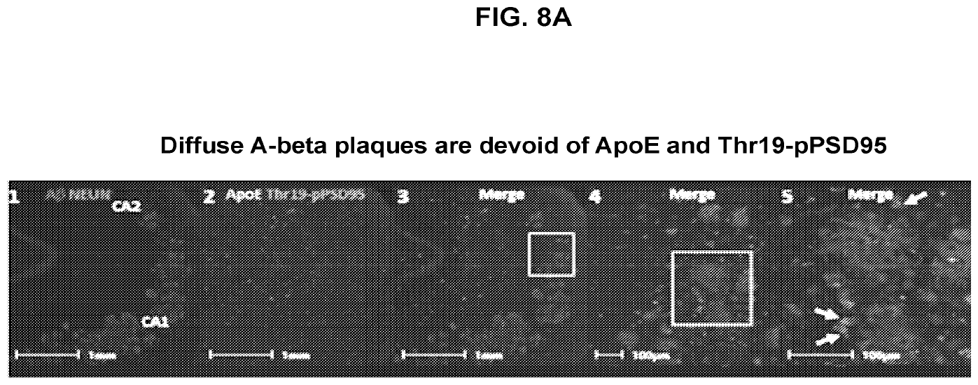

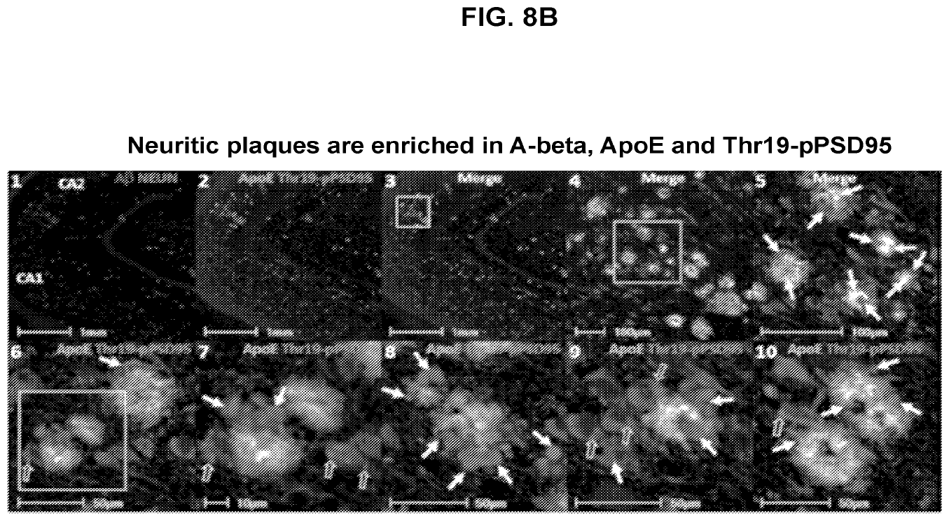

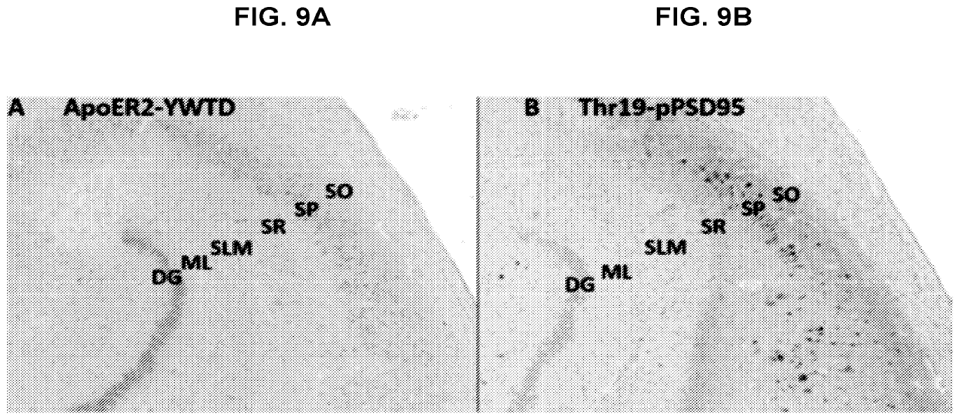

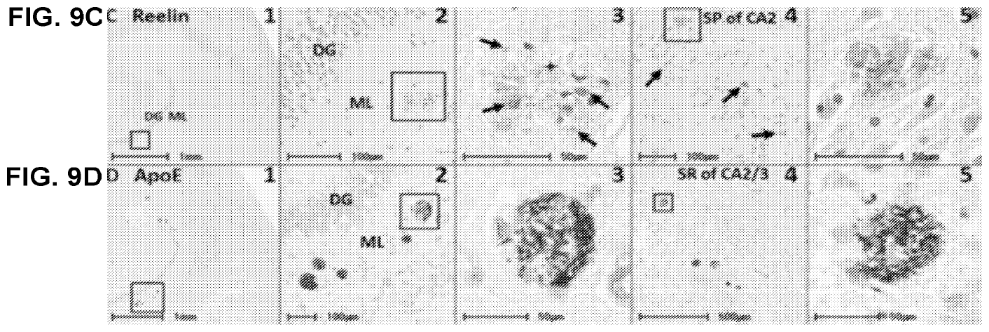

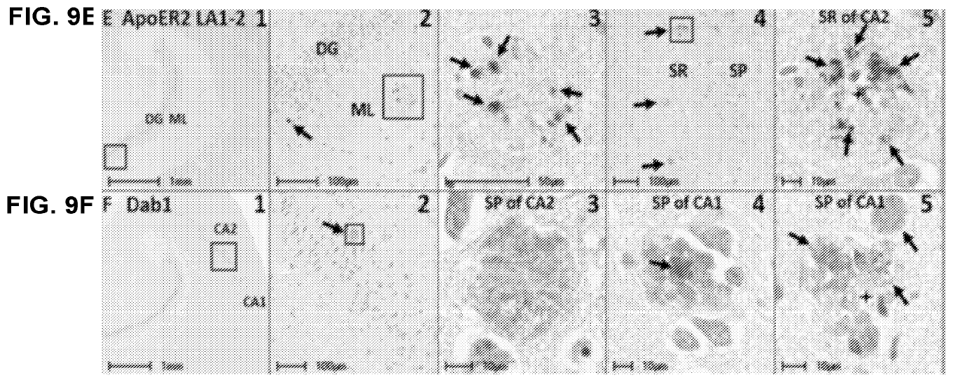

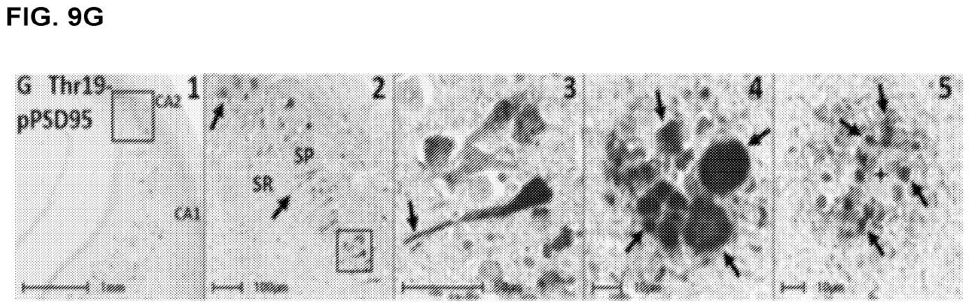

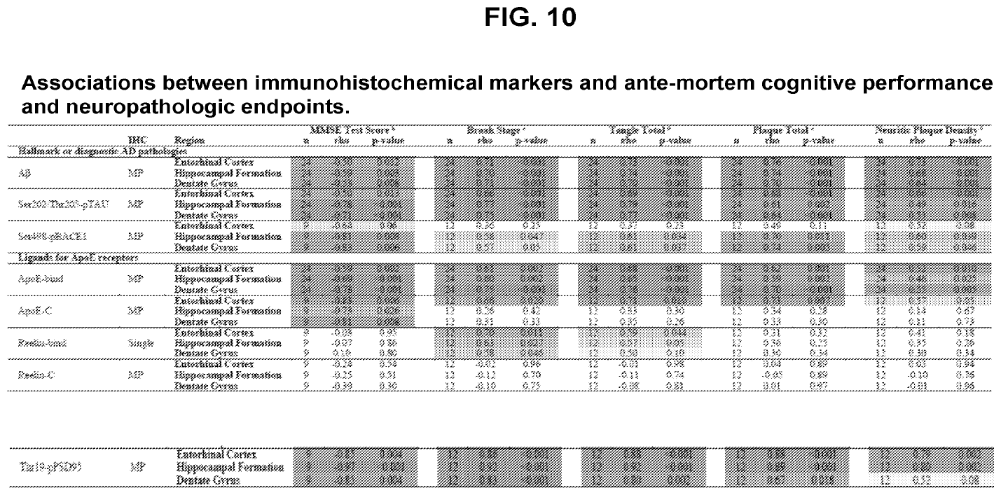

Thr19-pPSD95 accumulates within and around ApoE-enriched neuritic plaques in SAD. Postsynaptic ApoER2-PSD95-NMDA receptor complexes play a pivotal role of in LTP and memory in experimental models. Disruption of Reelin-ApoER2-Dab1-PI3K signaling activates GSK-362 , which in turn phosphorylates Tau. Activated GSK3β also phosphorylates PSD95 at Thr19 to induce disassembly of postsynaptic receptor complexes, suggesting that PSD95 and Tau phosphorylation may both be regulated by the Reelin-ApoER2 axis. Synapse disassembly and loss are major neurobiological correlates of memory loss, and Reelin deficiency has been shown to decrease PSD95 at the post-synapse, however Thr19-pPSD95 has not previously been investigated in AD. To address this gap, Thr19-pPSD95 was labeled in hippocampus and temporal cortex specimens from cases spanning the clinicopathological spectrum of sAD. Images from these regions in a representative Braak stage VI sAD case and non-AD control are provided in FIG. 2A. In sAD cases, striking accumulation of Thr19-pPSD95 were observed in the vicinity of neuritic plaques and within surrounding abnormal neurons in the molecular layer of dentate gyrus, CA1-2 subfields of the hippocampus, and the temporal cortex (FIG. 2A and FIG. 3). Non-AD controls had little or no expression of Thr19-pPSD95. MCI cases exhibited more modest expression of Thr19-pPSD95 that was mostly localized to abnormal neurons. Thr19-pPSD95 expression in all three annotated regions was strongly, positively correlated with Braak stage and Aβ plaque load, and negatively correlated with MMSE scores (FIG. 2B and FIG. 3A). MP-IHC revealed that Thr19-pPSD95 is primarily localized to MAP2-positive dendritic compartments (FIG. 2C). Unlike Aβ, Thr19-pPSD95 and ApoE are abundant in neuritic plaques but not diffuse plaques (FIG. 8A-8B). Additional IHC and MP-IHC examples demonstrating plaque-associated accumulation of Thr19-pPSD95, and spatial relations with other ApoE/Reelin-ApoER2-Dab1 pathway components are provided in FIG. 3.

Next MP-IHC was used to examine spatial, morphological, and cytoarchitectural relationships between individual ApoE/Reelin-ApoER2-Dab1 pathway markers in the neuritic plaque niche. An enlarged image of the molecular layer of the dentate gyrus from an advanced (Braak stage VI, ApoE3/3) sAD case containing extensive neuritic lesions is provided in FIG. 3B1-3B12. Single marker IHC revealed prominent Thr19-pPSD95-positive globular structures (black arrows in FIG. 3B1) amid numerous smaller fibrillar structures in this region. MP-IHC revealed that these globular structures co-express Thr19-pPSD95 and MAP2 (yellow arrows in FIG. 3B2), suggesting localization of Thr19-pPSD95 to swollen, dystrophic dendrites. Colocalization of Ser202/Thr205-pTau (yellow arrows in FIG. 3B3-3B4) within a subset of these Thr19-pPSD95 and MAP2-positive globular structures is consistent with somatodendritic localization of Tau. By contrast, expression of ApoE, HNE-ApoE, and Reelin was primarily localized to extracellular plaques (depicted by stars in FIG. 3B5-3B8), consistent with the concept of localized ApoE receptor disruption. As previously observed in cornu ammonis, HNE-ApoE exhibited only partial colocalization with native ApoE in the molecular layer of the dentate gyrus (FIG. 3B6-3B8). Single-marker IHC revealed that ApoER2 LA1-2 expression in this region was localized to fibrillar structures and discrete puncta (black arrows in FIG. 3B9). White arrows in FIG. 3B2 and FIG. 3B7 indicate that these ApoER2 LA1-2-positive puncta reside within dense, MAP2-positive dendritic arbors in the immediate vicinity of neuritic plaques. Singlemarker IHC revealed regional accumulation of Dab1, and strong expression of Tyr607-pPI3K and Thr508-pLIMK1 in unspecified globular structures that resemble dystrophic neurites (FIG. 3B10-3B12). These collective observations suggest that dendritic compartments and postsynaptic densities within the terminal zones of the perforant path are a major locus for ApoE/Reelin-ApoER2-Dab1-PI3K-LIMK1-Tau axis pathologies in sAD, with Thr19-pPSD95 accumulation suggesting that these axis pathologies culminate in synaptic disassembly.

In the present study, striking immunoreactivity for Thr19-pPSD95 was observed in MAP2-positive dystrophic dendrites in proximity to ApoE-and Reelin-enriched plaques and adjacent neurons in regions affected by sAD (FIGS. 2-3), major accumulation of Thr19-pPSD95 in sAD cases, and strong positive correlations between Thr19-pPSD95 and histological progression and antemortem cognitive deficits (FIGS. 2-3). This is the first demonstration of Thr19-pPSD95 immunolabeling in human entorhinal-hippocampal structures, and the first evidence linking Thr19-pPSD95 to AD. These observations suggest that compromised Reelin-ApoE receptor-Dab1-PI3K signaling could enhance GSK3β-mediated phosphorylation of both Tau and PSD95, leading to parallel destabilization of the microtubule cytoskeleton and postsynaptic receptor clusters within excitatory synapses, respectively. Anticipated functional correlates of such alterations may include enhanced LTD and synaptic dysfunction, while long-term pathological correlates may include synaptic loss with accumulation of Ser202/Thr205-pTau and Thr19-pPSD95, as observed in this study. Because synapse dysfunction and loss are implicated in memory deficits in sAD, Thr19-phosphorylation of PSD95 should be studied further as both a potential underlying mechanism and biomarker for AD.

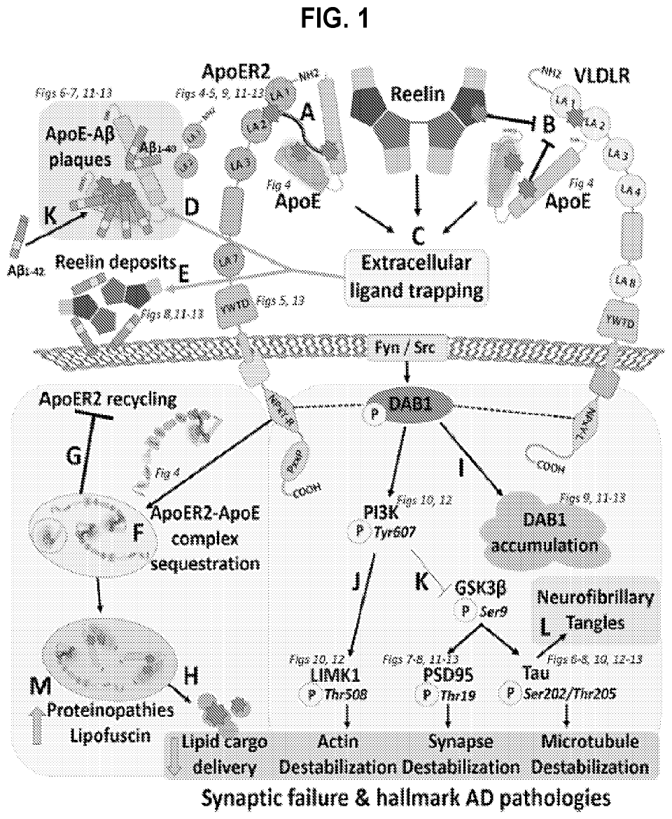

Postsynaptic receptor complexes as a locus for ApoE/Reelin-ApoER2-Dab1 pathologies in sAD. The splice variant of ApoER2 that enables formation of postsynaptic ApoER2-PSD95-NMDA receptor complexes helps account for the pivotal role of ApoER2 in LTP and memory. In the present study, colocalization of Thr19pPSD95 and MAP2 was observed (FIG. 2-3), and localized enrichment of ApoER2, Dab1, Thr508-pLIMK1, Tyr607-pPI3K and Ser202/Thr205-pTau in the vicinity of ApoE-, Reelin- and Aβ-enriched neuritic plaques. ApoER2 could serve as a molecular integrator, transmitting signals from extracellular Reelin and ApoE (and Aβ) to postsynaptic elements of excitatory synapses by regulating ApoER2-PSD95-NMDA-Tau complex integrity, Reelin-ApoER2-Dab1 signal transduction, and ultimately the balance between LTP and LTD. A model (FIG. 1) wherein chronic disruption of ApoER2 at the post-synapse leads to both synapse failure and the hallmark sAD pathologies including extracellular ApoE-Aβ complexes and intraneuronal neurofibrillary tangles.

### Example 2: Immunohistochemistry with Thr19-phospho-PSD95

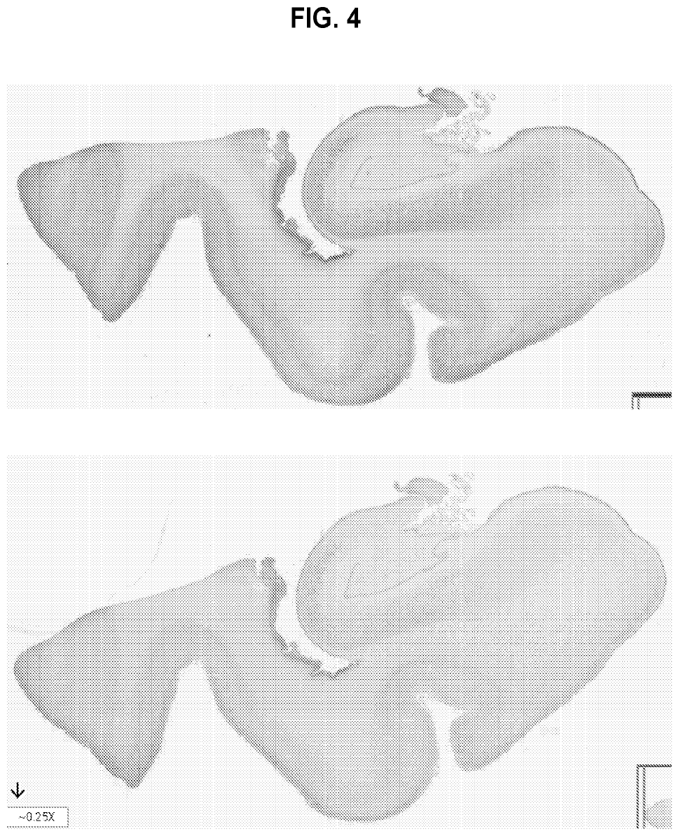

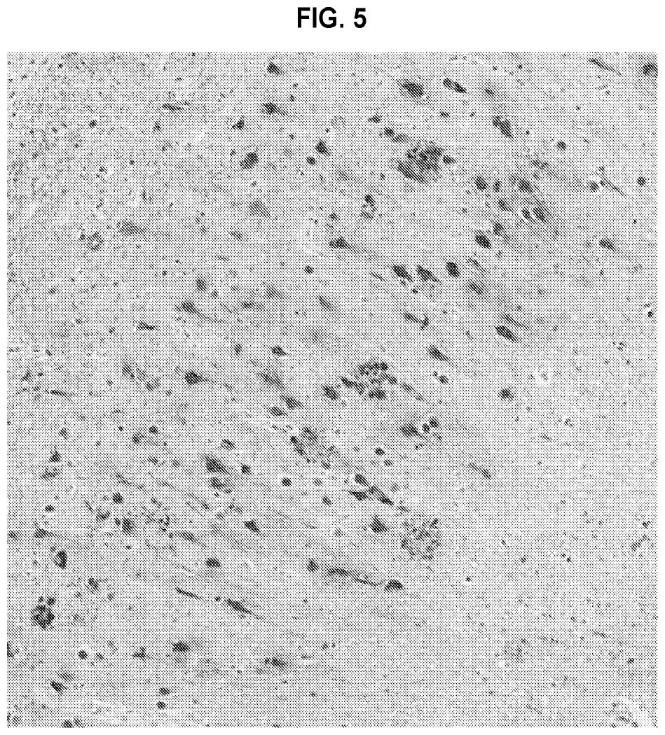

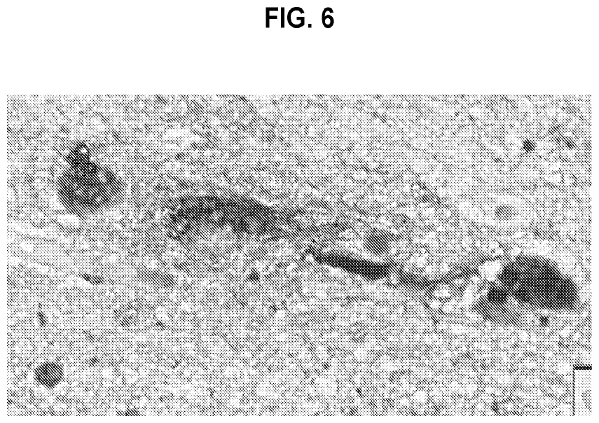

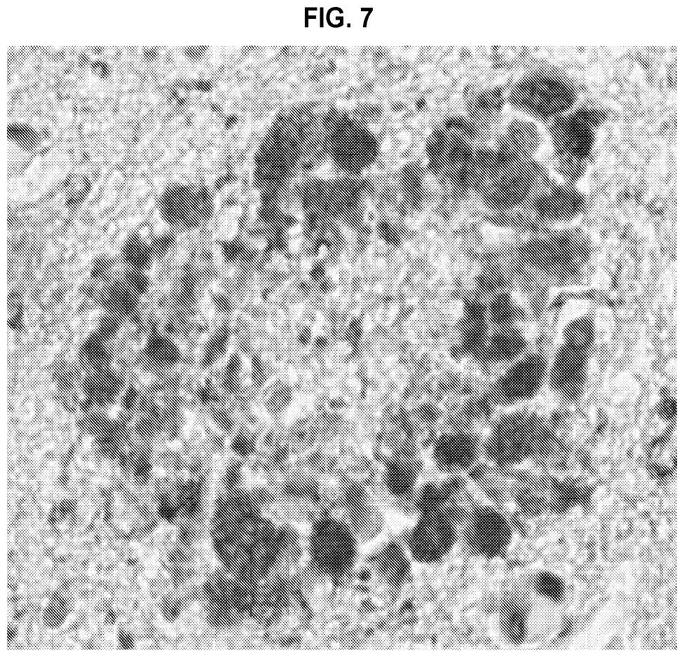

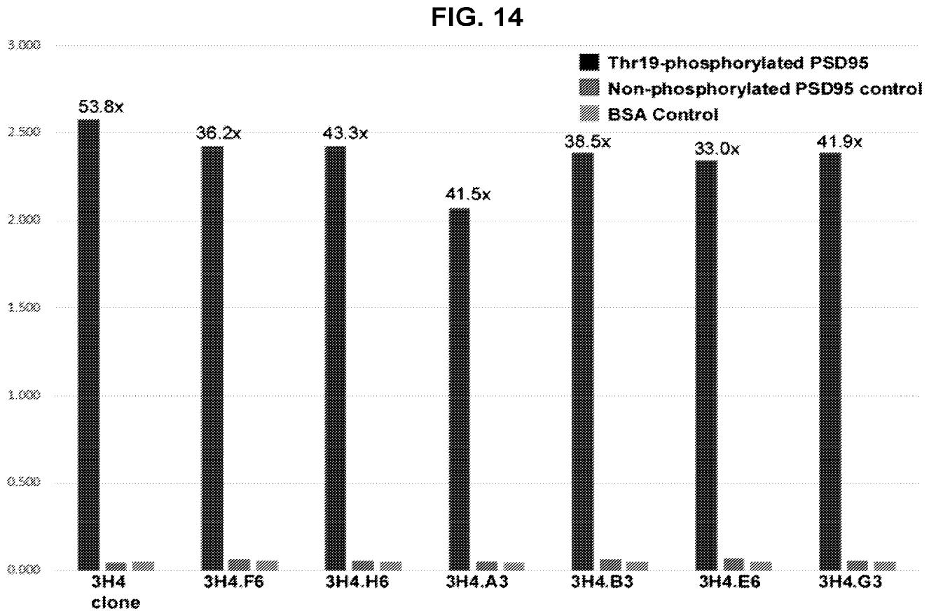

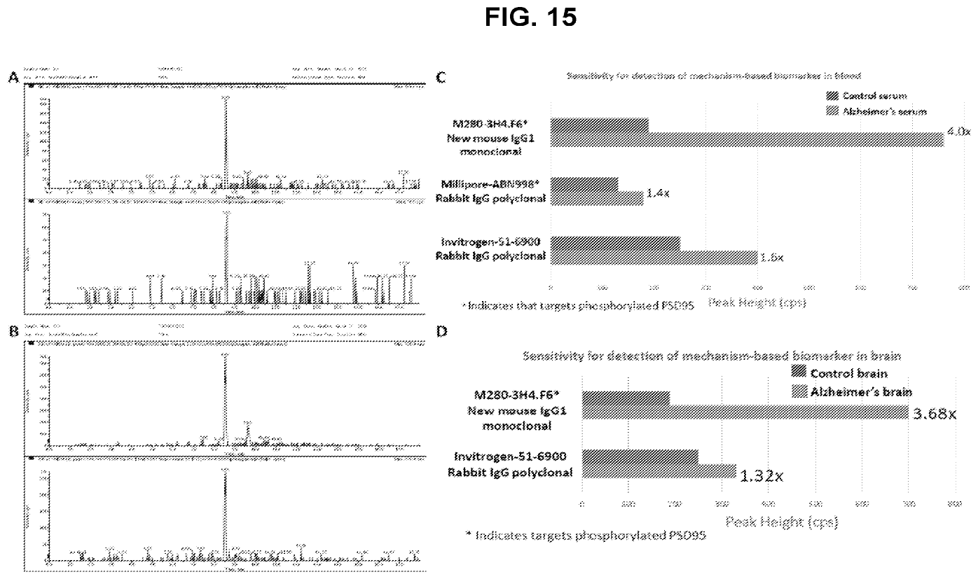

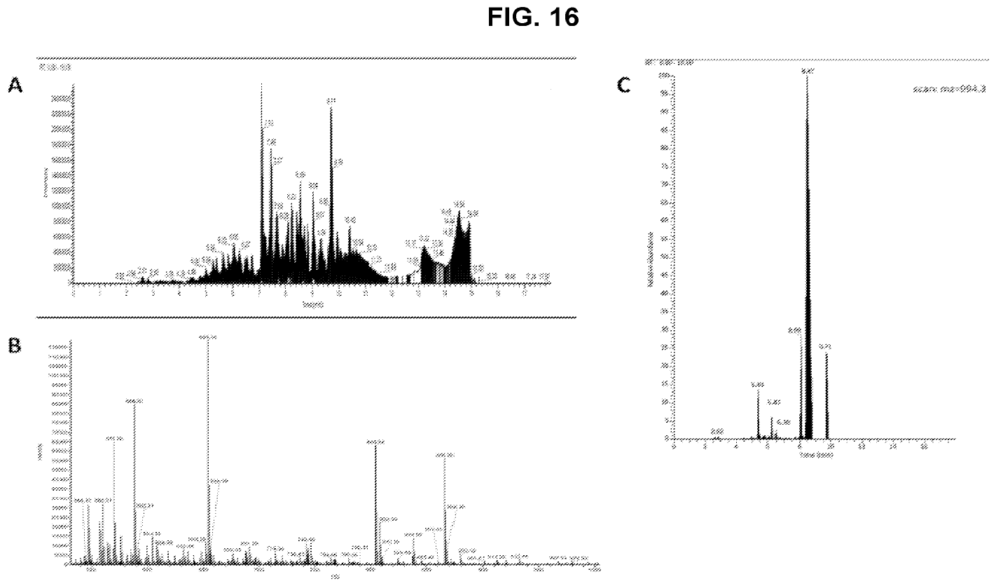

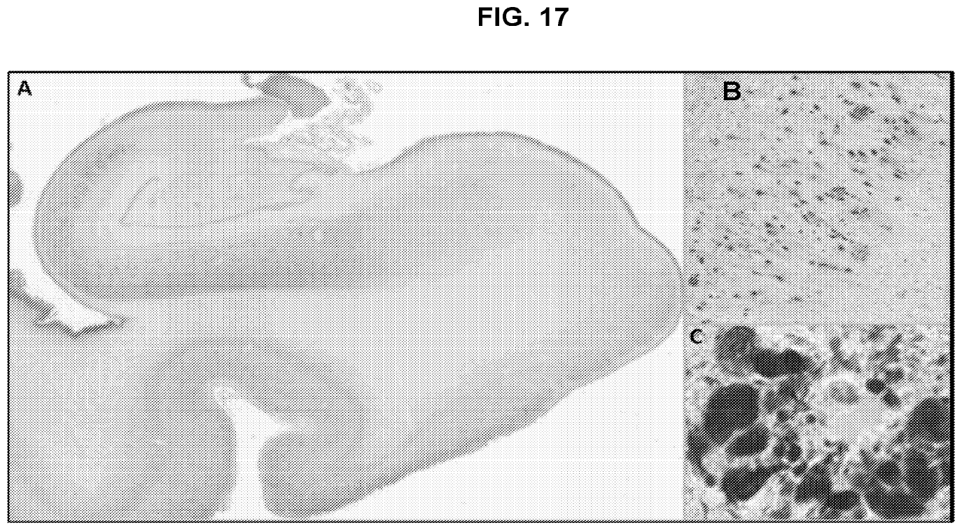

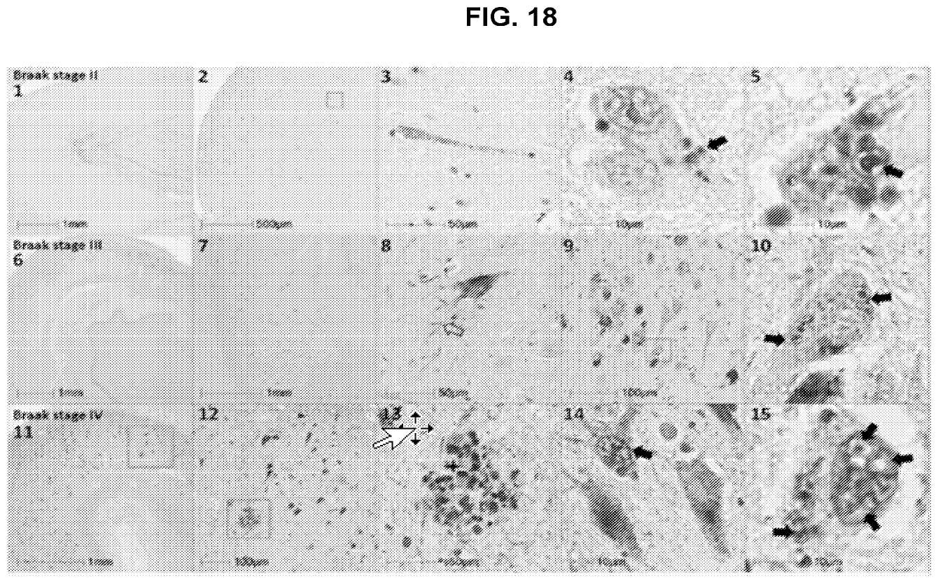

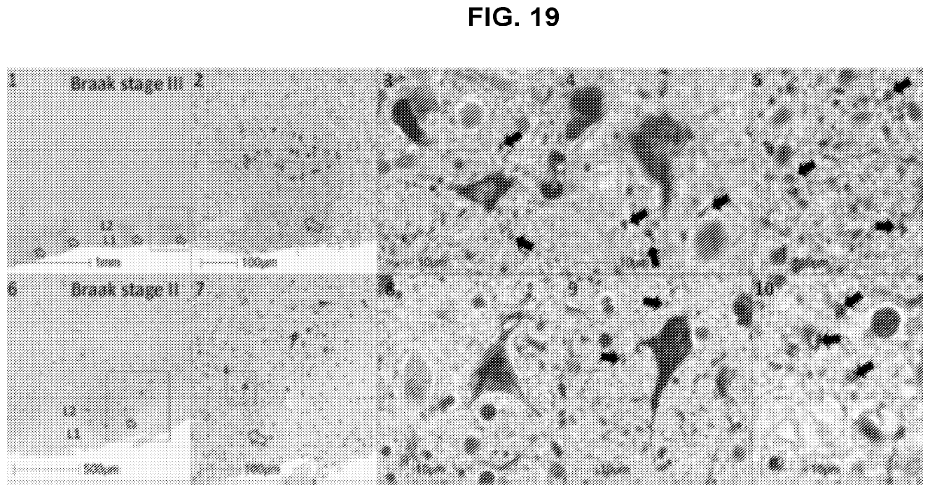

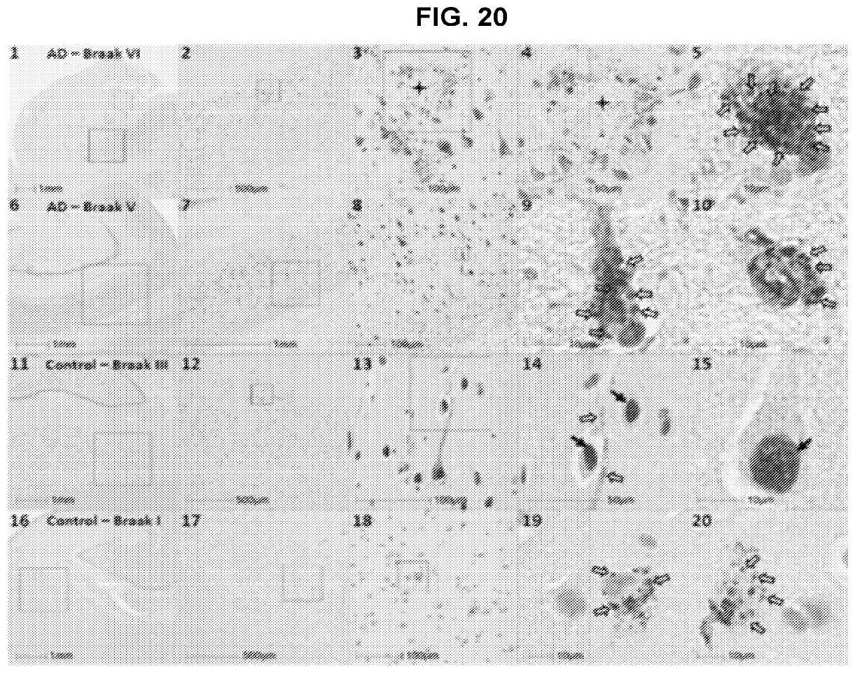

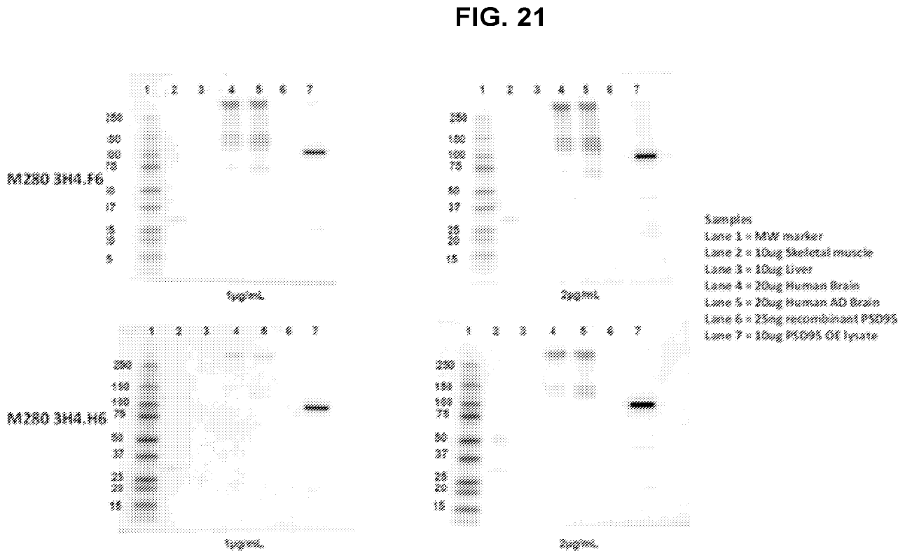

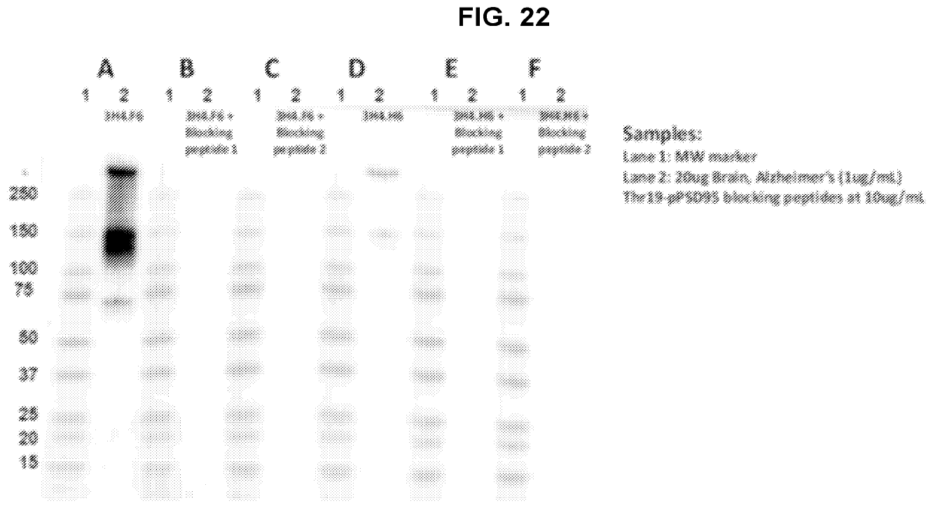

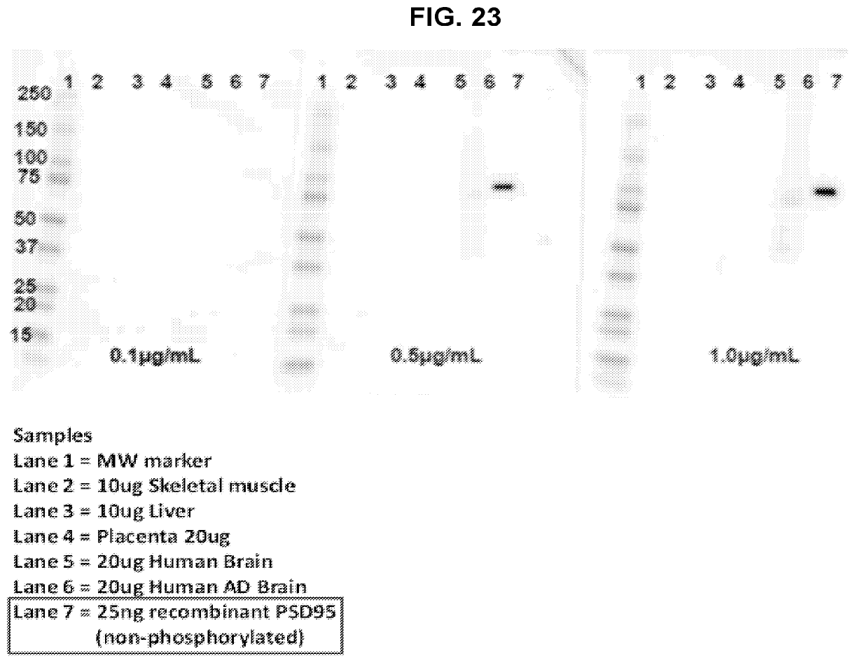

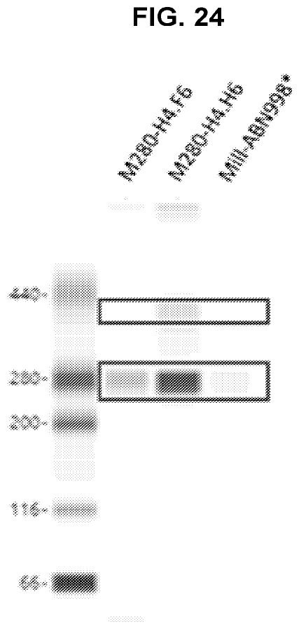

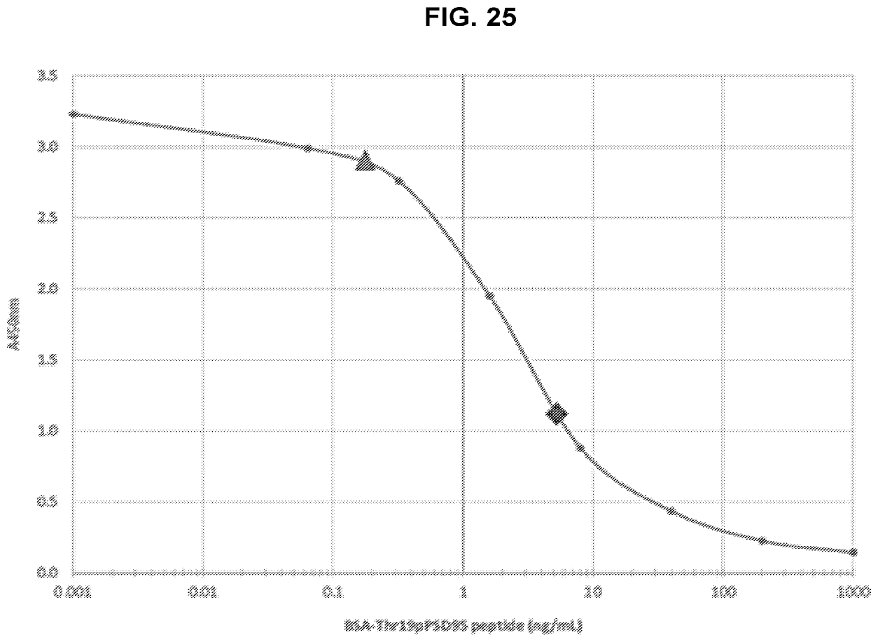

Human brain sections were tested with the anti-Thr19-phospho-PSD95 antibodies as disclosed herein. As shown in FIGS. 4-7, the antibodies specific to Thr19-phospho-PSD95 specifically recognizes Threonine-19 phosphorylated PSD95 in brains from Alzheimer's disease (neurofibrillary tangles and neuropil threads) by immunohistochemistry. The anti-Thr19-phospho-PSD95 antibodies selectively detect Thr19-phospho-PSD95 in affected neurons and neuritic plaques in the entorhinal-hippocampal memory system (FIG. 4), and the cornu ammonis (FIG. 5) in Alzheimer's disease. The antibodies also showed only a very low level of background staining in all tissues tested. See also FIGS. 14-25.

### Example 3: M312 Antibodies Selectively Detect Dual Thr19 and Ser25-Phosphorylated PSD95

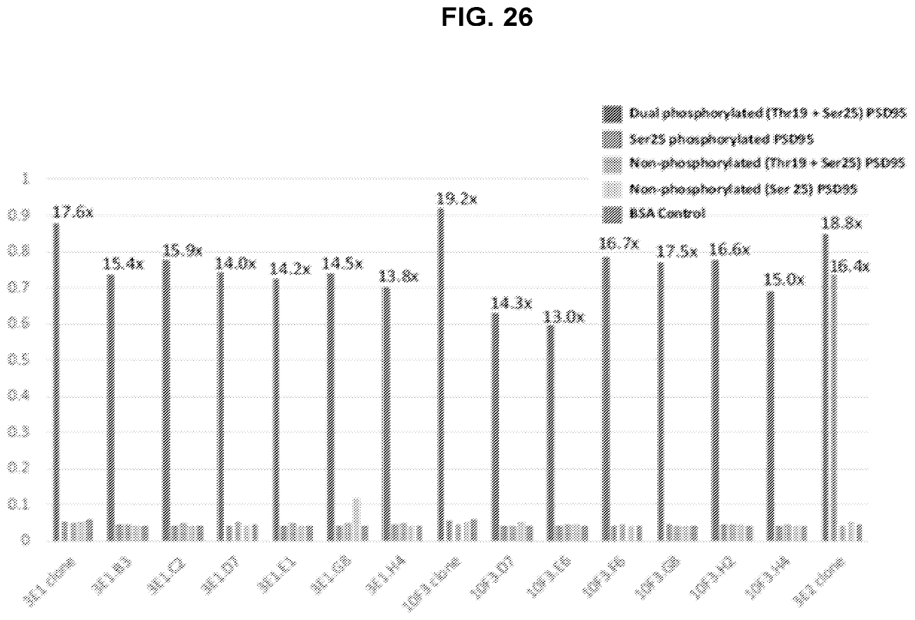

ELISA assays showed that mouse IgG1 monoclonal antibodies generated from parent clones 3E1 and 10F3 and several subclones including M312-3E1.D7 and M312-10F3.H2 selectively detected dual phosphorylated (Thr19+Ser25) PSD95 with minimal or no detection of the corresponding single phosphorylated (Ser25) sequence, non-phosphorylated PSD95 sequences, or a BSA control. Each clone and subclone had >13-fold higher affinity for the Thr19-phosphorylated PSD95 compared to both controls. M312-3E2 detected both dual phosphorylated (Thr19+Ser25) PSD95 and the Ser25 phosphorylated PSD95 but not the control (see FIG. 26). Coating antigen concentration: 2 ug/mL. Blocking buffer: 0.1% BSA-PBS. Primary antibody: fusion supernatant (neat). Secondary antibody: Goat anti-Mouse IgG at 1:10,000 dilution.

Having described the disclosure in detail and by reference to specific embodiments thereof, it will be apparent that modifications and variations are possible without departing from the scope of the disclosure defined in the appended claims. More specifically, although some aspects of the present disclosure are identified herein as particularly advantageous, it is contemplated that the present disclosure is not necessarily limited to these particular aspects of the disclosure.

SEQUENCES:

Ac-CDED(pT)PPLE (SEQ ID NO: 1)
CDED(pT)PPLE (SEQ ID NO: 2)
DED(pT)PPLE (SEQ ID NO: 3)
D(pT)PPLEHSP (SEQ ID NO: 4)
D(pT)PPLEH(ps)P (SEQ ID NO: 5)
D(pT)PPLEHSPA (SEQ ID NO: 6)
D(pT)PPLEH(pS)PA (SEQ ID NO: 7)
D(pT)PPLEHSPAH (SEQ ID NO: 8)
D(pT)PPLEH(pS)PAH (SEQ ID NO: 9)
ED(pT)PPLEHSPA (SEQ ID NO: 10)
ED(pT)PPLEH(pS)PA (SEQ ID NO: 11)
ED(pT)PPLEHSPAH (SEQ ID NO: 12)
ED(pT)PPLEH(pS)PAH (SEQ ID NO: 13)
DED(pT)PPLEHSPA (SEQ ID NO: 14)
DED(pT)PPLEH(pS)PA (SEQ ID NO: 15)
DED(pT)PPLEHSPAH (SEQ ID NO: 16)
DED(pT)PPLEH(pS)PAH (SEQ ID NO: 17)
Ac-CDED(pT)PPLEH(pS)PAH (SEQ ID NO: 49)
CDED(pT)PPLEH(pS)PAH (SEQ ID NO: 50)
KYRYQDED(pT)PPLEHSPAHLP (SEQ ID NO: 18);
KYRYQDED(pT)PPLEH(pS)PAHLP (SEQ ID NO: 19)
YRYQDED(pT)PPLEHSPAHLP (SEQ ID NO: 20)
YRYQDED(pT)PPLEH(pS)PAHLP (SEQ ID NO: 21)
RYQDED(pT)PPLEHSPAHLP (SEQ ID NO: 22)
RYQDED(pT)PPLEH(pS)PAHLP (SEQ ID NO: 23)
YQDED(pT)PPLEHSPAHLP (SEQ ID NO: 24)
YQDED(pT)PPLEH(pS)PAHLP (SEQ ID NO: 25)
QDED(pT)PPLEHSPAHLP (SEQ ID NO: 26)
QDED(pT)PPLEH(pS)PAHLP (SEQ ID NO: 27)
KYRYQDED(pT)PPLEHSPAHL (SEQ ID NO: 28)
KYRYQDED(pT)PPLEH(pS)PAHL (SEQ ID NO: 29)
KYRYQDED(pT)PPLEHSPAH (SEQ ID NO: 30)
KYRYQDED(pT)PPLEH(pS)PAH (SEQ ID NO: 31)
KYRYQDED(pT)PPLEHSPA (SEQ ID NO: 32)
KYRYQDED(pT)PPLEH(pS)PA (SEQ ID NO: 33)
KYRYQDED(pT)PPLEH(pS)P (SEQ ID NO: 34)
KYRYQDED(pT)PPLEHSP (SEQ ID NO: 35)
KYRYQDED(pT)PPLEHS (SEQ ID NO: 36)
KYRYQDED(pT)PPLEH(pS) (SEQ ID NO: 37)
KYRYQDED(pT)PPLEH (SEQ ID NO: 38)
M280 SEQUENCES
HEAVY CHAIN (HC)
DNA Sequence
gaggtgcggcttgttgagtctggtggaggattggtgcagcctaaagggtcattgaaaatctcatgtgcagcctctgg
attcaccttcaacacctacgccatgaactgggtccgccaggctccaggaaagggtttggaatggattgctcgcataa
gaagtaaaagtaataattatgcaacatattatgacgattcagtgaaagacaggttcaccatctccagagatgattca
caaagcatgctctatctgcaaatgaacaacttgaaaactgaggacacagccatgtattactgtgtgagacgggggga
tggtcactacatctggtttgcttactggggccaagggactctggtcactgtctctgca (SEQ ID NO: 39)
Predicted Protein Sequence
Complementarity determining regions (CDRs) are bolded and underlined.
EVRLVESGGGLVQPKGSLKISCAASGFTFNTYAMNWVRQAPGKGLEWIARIRSKSNNYATYYDDSVKDRFTISRDDS
QSMLYLQMNNLKTEDTAMYYCVRRGDGHYIWFAYWGQGTLVTVSA (SEQ ID NO: 40)

| | | |
|---|---|---|
| HC-CDR1 | GFTFNTYA | (SEQ ID NO: 41) |
| HC-CDR2 | IRSKSNNYAT | (SEQ ID NO: 42) |
| HC-CDR3 | VRRGDGHYIWFAY | (SEQ ID NO: 43) |

LIGHT CHAIN (LC)
DNA Sequence
gatgttttgatgacccaaactccactctccctgcctgtcagtcttggagatcaagcctccatctcttgcagatctag
tcagagcattgtaaatagtaatggaaacacctatttagaatggtacctgcagaaaccaggccagtctccaaagctcc
tgatctacaaagtttccaaccgattttctggggtcccagacaggttcagtggcagtggatcagggacagatttcaca
ctcaagatcagcagagtggaggctgaggatctgggagtttattactgctttcaaggttcacatgttccgctcacgtt
cggtgctgggaccaagctggagctgaaa (SEQ ID NO: 44)
Predicted Protein Sequence
Complementarity determining regions (CDRs) are bolded and underlined.
DVLMTQTPLSLPVSLGDQASISCRSSQSIVNSNGNTYLEWYLQKPGQSPKLLIYKVSNRFSGVPDRFSGSGSGTDFT
LKISRVEAEDLGVYYCFQGSHVPLTFGAGTKLELK (SEQ ID NO: 45)

| | | |
|---|---|---|
| LC-CDR1 | QSIVNSNGNTY | (SEQ ID NO: 46) |
| LC-CDR2 | KVS | (SEQ ID NO: 47) |
| LC-CDR3 | FQGSHVPLT | (SEQ ID NO: 48) |

M312 Sequence (3E1-D7)
Heavy Chain (VH) [IgG1]
gaggtgcagcttgttgagactggtggaggattggtgcagcctaaagggtcattgaaactctcatgtgcagcctctgg
attcaccttcaataccaatgccatgaattgggtccgccaggctccaggaaagggtttggaatggattgctcgcataa
gaactaaaagtaataattatggaacatattatgccgattcagtgaaagacaggttcaccatttccagagatgattca
caaaacatactttatctgcaaatgaacaacttgaaaactgaggacacagccatgtattactgtgtgagagggaagat
caactatgattacgggtttggttactggggccaagggactctggtcactgtctctgcag (SEQ ID NO: 51)
Predicted Protein Sequence (VH)
Complementarity determining regions (CDRs) are bolded and underlined.
EVQLVETGGGLVQPKGSLKLSCAASGFTFNTNAMNWVRQAPGKGLEWIARIRTKSNNYGTYYADSVKDRFTISRDDS
QNILYLQMNNLKTEDTAMYYCVRGKINYDYGFGYWGQGTLVTVSA (SEQ ID NO: 52)

| | | |
|---|---|---|
| HC-CDR1 | GFTFNTNA | (SEQ ID NO: 53) |
| HC-CDR2 | IRTKSNNYGT | (SEQ ID NO: 54) |
| HC-CDR3 | VRGKINYDYGFGY | (SEQ ID NO: 55) |

Light Chain (VL) [Kappa]
gatgttttgatgacccaaactccactctccctgcctgtcagtcttggagatcaagcctccatctcttgcagatctag
tcagagcattgtacatagtaatggaaacacctatttagaatggttcctgcagaaaccaggccagtctccaaagctcc
tgatctacaaagtttccaaccgattttctggggtcccagacaggttcagtggcagtggatcagggactgatttcaca
ctcaagatcagcagaatggaggctgaggatctgggagtttattattgttttcaaggttcacatgttccttacacgtt
cggaggggggaccaagctggaaataaaac (SEQ ID NO: 56)
Predicted Protein Sequence (VL)
Complementarity determining regions (CDRs) are bolded and underlined.
DVLMTQTPLSLPVSLGDQASISCRSSQSIVHSNGNTYLEWFLQKPGQSPKLLIYKVSNRFSGVPDRFSGSGSGTDFT
LKISRMEAEDLGVYYCFQGSHVPYTFGGGTKLEIK (SEQ ID NO: 57)

| | | |
|---|---|---|
| LC-CDR1 | QSIVHSNGNTY | (SEQ ID NO: 58) |
| LC-CDR2 | KVS | (SEQ ID NO: 47) |
| LC-CDR3 | FQGSHVPYT | (SEQ ID NO: 59) |

M312 Sequence (10F3-H2)
Heavy Chain (VH) [IgG1]
gaggtgcagcttgttgagactggtggaggattggtgcagcctaaagggtcattgaaactctcatgtgcagcctctgg
attcaccttcaataccaatgccatgaattgggtccgccaggctccaggaaagggtttggaatggattgctcgcataa
gaactaaaagtaatcattatggaacatattatgccgattcagtgaaagacaggttcaccatttccagagatgattca
caaaacatactttatctgcaaatgaacaacttgaaaactgaggacacagccatgtattactgtgtgagaggccagat
caactatgattacgggtttggttactggggccaagggactctggtcactgtctctgcag (SEQ ID NO: 60)
Predicted Protein Sequence (VH)
Complementarity determining regions (CDRs) are bolded and underlined.
EVQLVETGGGLVQPKGSLKLSCAASGFTFNTNAMNWVRQAPGKGLEWIARIRTKSNHYGTYYADSVKDRFTISRDDS
QNILYLQMNNLKTEDTAMYYCVRGQINYDYGFGYWGQGTLVTVSA (SEQ ID NO: 61)

| | | |
|---|---|---|
| HC-CDR1 | GFTFNTNA | (SEQ ID NO: 53) |
| HC-CDR2 | IRTKSNHYGT | (SEQ ID NO: 62) |
| HC-CDR3 | VRGQINYDYGFGY | (SEQ ID NO:63) |

Light Chain (VL) [Kappa]
DNA Sequence
gatgttttgatgacccaaactccactctccctgcctgtcagtcttggagatcaagcctccatctcttgcagatctag
tcagaacattgtacatagtaatggaaacacctatttagaatggtacctgcagaaaccaggccagtctccaaagctcc
tgatctacaaagtttccaaccgattttctggggtcccagacaggttcagtggcagtggatcagggactgatttcaca
ctcaagatcagcagaatggaggctgaggatctgggagtttattattgttttcaaggttcacatgttccttacacgtt
cggaggggggaccaagctggaaataaaac (SEQ ID NO: 64)
Predicted Protein Sequence (VL)
Complementarity determining regions (CDRs) are bolded and underlined.
DVLMTQTPLSLPVSLGDQASISCRSSQNIVHSNGNTYLEWYLQKPGQSPKLLIYKVSNRFSGVPDRFSGSGSGTDFT
LKISRMEAEDLGVYYCFQGSHVPYTFGGGTKLEIK (SEQ ID NO: 65)

| | | |
|---|---|---|
| LC-CDR1 | QNIVHSNGNTY | (SEQ ID NO: 66) |
| LC-CDR2 | KVS | (SEQ ID NO: 47) |
| LC-CDR3 | FQGSHVPYT | (SEQ ID NO: 59) |

1. An immunoglobulin or antigen binding fragment thereof that specifically binds postsynaptic density (PSD95) phosphorylated at threonine 19, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 41, the VH-CDR2 comprises the amino acid sequence set forth in SEQ ID NO: 42, the VH-CDR3 comprises the amino acid sequence set forth in SEQ ID NO: 43, the VL-CDR1 comprises the amino acid sequence set forth in SEQ ID NO: 46, the VL-CDR2 comprises the amino acid sequence set forth in SEQ ID NO: 47, and the VL-CDR3 comprises the amino acid sequence set forth in SEQ ID NO: 48.

2. The immunoglobulin or antigen binding fragment thereof of claim 1, wherein the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 40.

3. The immunoglobulin or antigen binding fragment thereof of claim 2, wherein the VH comprises the amino acid sequence of SEQ ID NO: 40.

4. The immunoglobulin or antigen binding fragment thereof of claim 1, wherein the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 45.

5. The immunoglobulin or antigen binding fragment thereof of claim 4, wherein the VL comprises the amino acid sequence of SEQ ID NO: 45.

6. The immunoglobulin or antigen binding fragment thereof of claim 1, wherein the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 40, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 45.

7. The immunoglobulin or antigen binding fragment thereof of claim 1, wherein the VH comprises the amino acid sequence set forth in SEQ ID NO: 40, and the VL comprises the amino acid sequence set forth in SEQ ID NO: 45.

8. The immunoglobulin or antigen binding fragment thereof of claim 1, which binds to PSD-95 phosphorylated at threonine 19 with a dissociation constant (Kd) value of at least 10⁻⁸ M.

9. The immunoglobulin or antigen binding fragment thereof of claim 1, which is a monoclonal antibody.

10. The immunoglobulin or antigen binding fragment thereof of claim 1, which is an antibody fragment that binds to PSD95 phosphorylated at threonine 19.

11. A nucleic acid, comprising a nucleotide sequence encoding the amino acid sequence of SEQ ID NO: 40 and 45.

12. A vector, comprising the nucleic acid of claim 11.

13. A host cell, comprising the expression vector of claim 12.

14. A method of diagnosis of a neurological disorder in a subject using the immunoglobulin or antigen binding fragment thereof of claim 1.

15. The method of claim 14, wherein the diagnosis is in vitro and/or in vivo.

16. A method for diagnosing a neurological disorder in a subject, comprising detecting in a sample from the subject threonine 19 phosphorylated PSD-95, wherein the sample is contacted with the immunoglobulin or antigen binding fragment thereof of claim 1.

17. The method of claim 16, wherein the diagnosis is in vitro and/or in vivo.

18. The method of claim 14, wherein the method comprises an assay selected from the group consisting of: a radioimmunoassay, an immunohistochemistry assay, a competitive-binding assay, a Western Blot analysis, an ELISA assay, a two-dimensional gel electrophoresis, an enzyme immunoassay, a sandwich immunoassay, an immunodiffusion assay, an immunoradiometric assay, a fluorescent immunoassay, and an immunoelectrophoresis assay.

19. The method of claim 14, wherein the neurological disorder is selected from the group consisting of acute spinal cord injury, Alzheimer's disease, Amyotrophic Lateral Sclerosis (ALS), anxiety, ataxia, attention-deficit disorder, autism, behavior disorders (e.g., Attention Deficit Hyperactivity Disorder (ADHD), Behavioral Addiction, Conduct Disorder, Obsessive-compulsive disorder (OCD), and Oppositional Defiant Disorder (ODD)) Bell's palsy, brain tumors, cerebral aneurysm, cognitive decline, depression, epilepsy (and seizures), Guillain-Barre syndrome, headache, head injury, hydrocephalus, intellectual disorders (e.g., Down syndrome, fragile x syndrome, fetal alcohol syndrome, and Prader-Willi syndrome) meningitis, multiple sclerosis, muscular dystrophy, Parkinson's disease, schizophrenia, or stroke.

20. The method of claim 14, wherein the sample is selected from the group consisting of blood, serum, plasma, urine, feces, respiratory secretions, exosome, cerebrospinal fluid, saliva, and brain tissue.

21-22. (canceled).

23. An immunoglobulin or antigen binding fragment thereof that specifically binds postsynaptic density (PSD95) phosphorylated at both threonine 19 and serine 25, comprising a variable heavy chain region (VH) and a variable light chain region (VL), wherein the VH comprises a VH complementary determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 53, the VH-CDR2 comprises the amino acid sequence set forth in SEQ ID NO: 54, the VH-CDR3 comprises the amino acid sequence set forth in SEQ ID NO: 55, the VL-CDR1 comprises the amino acid sequence set forth in SEQ ID NO: 58, the VL-CDR2 comprises the amino acid sequence set forth in SEQ ID NO: 47, and the VL-CDR3 comprises the amino acid sequence set forth in SEQ ID NO: 59.

24-32. (canceled).

33. A nucleic acid, comprising a nucleotide sequence encoding the amino acid sequence of SEQ ID NO: 52 and 57.

34. A vector, comprising the nucleic acid of claim 33.

35. A host cell, comprising the expression vector of claim 34.

36. A method of diagnosis of a neurological disorder in a subject using the immunoglobulin or antigen binding fragment thereof of claim 23.

37. The method of claim 36, wherein the diagnosis is in vitro and/or in vivo.

38. A method for diagnosing a neurological disorder in a subject, comprising detecting in a sample from the subject both threonine 19 and serine 25 phosphorylated PSD-95, wherein the sample is contacted with the immunoglobulin or antigen binding fragment thereof of claim 23.

39. The method of claim 38, wherein the diagnosis is in vitro and/or in vivo.

40-44. (canceled).

45. An immunoglobulin or antigen binding fragment thereof that specifically binds postsynaptic density (PSD95) phosphorylated at both threonine 19 and serine 25, comprising a variable heavy chain region (VH) and a variable light chain region (VL), wherein the VH comprises a VH complementary determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 53, the VH-CDR2 comprises the amino acid sequence set forth in SEQ ID NO: 62, the VH-CDR3 comprises the amino acid sequence set forth in SEQ ID NO: 63, the VL-CDR1 comprises the amino acid sequence set forth in SEQ ID NO: 66, the VL-CDR2 comprises the amino acid sequence set forth in SEQ ID NO: 47, and the VL-CDR3 comprises the amino acid sequence set forth in SEQ ID NO: 59.

46-54. (canceled).

55. A nucleic acid, comprising a nucleotide sequence encoding the amino acid sequence of SEQ ID NO: 61 and 65.

56. A vector, comprising the nucleic acid of claim 55.

57. A host cell, comprising the expression vector of claim 56.

58. A method of diagnosis of a neurological disorder in a subject using the immunoglobulin or antigen binding fragment thereof of claim 45.

59. The method of claim 58, wherein the diagnosis is in vitro and/or in vivo.

60. A method for diagnosing a neurological disorder in a subject, comprising detecting in a sample from the subject both threonine 19 and serine 25 phosphorylated PSD-95, wherein the sample is contacted with the immunoglobulin or antigen binding fragment thereof of claim 45.

61. The method of claim 60, wherein the diagnosis is in vitro and/or in vivo.

62-66. (canceled).

67. An immunoglobulin or an antigen binding fragment that binds to protein in the postsynaptic density (PSD95) phosphorylated at threonine 19, at serine 25, and/or at both threonine 19 and serine 25.

68. An immunoglobulin or an antigen binding fragment that binds to protein in the postsynaptic density (PSD95) phosphorylated at threonine 19, at serine 25, and/or at both threonine 19 and serine 25, wherein the immunoglobulin or the antigen binding fragment is obtained by immunization with a polypeptide having an amino acid sequence selected from:
(SEQ ID NO: 1)
Ac-CDED(pT)PPLE;
(SEQ ID NO: 2)
CDED(pT)PPLE;
(SEQ ID NO: 3)
DED(pT)PPLE;
(SEQ ID NO: 4)
D(pT)PPLEHSP;
(SEQ ID NO: 5)
D(pT)PPLEH(pS)P;
(SEQ ID NO: 6)
D(pT)PPLEHSPA;
(SEQ ID NO: 7)
D(pT)PPLEH(pS)PA;
(SEQ ID NO: 8)
D(pT)PPLEHSPAH;
(SEQ ID NO: 9)
D(pT)PPLEH(pS)PAH;
(SEQ ID NO: 10)
ED(pT)PPLEHSPA;
(SEQ ID NO: 11)
ED(pT)PPLEH(pS)PA;
(SEQ ID NO: 12)
ED(pT)PPLEHSPAH;
(SEQ ID NO: 13)
ED(pT)PPLEH(pS)PAH;
(SEQ ID NO: 14)
DED(pT)PPLEHSPA;
(SEQ ID NO: 15)
DED(pT)PPLEH(pS)PA;
(SEQ ID NO: 16)
DED(pT)PPLEHSPAH;
(SEQ ID NO: 17)
DED(pT)PPLEH(pS)PAH;
(SEQ ID NO: 49)
Ac-CDED(pT)PPLEH(pS)PAH;
(SEQ ID NO: 50)
CDED(pT)PPLEH(pS)PAH;
(SEQ ID NO: 18)
KYRYQDED(pT)PPLEHSPAHLP;
(SEQ ID NO: 19)
KYRYQDED(pT)PPLEH(pS)PAHLP;
(SEQ ID NO: 20)
YRYQDED(pT)PPLEHSPAHLP;
(SEQ ID NO: 21)
YRYQDED(pT)PPLEH(pS)PAHLP;
(SEQ ID NO: 22)
RYQDED(pT)PPLEHSPAHLP;
(SEQ ID NO: 23)
RYQDED(pT)PPLEH(pS)PAHLP;
(SEQ ID NO: 24)
YQDED(pT)PPLEHSPAHLP;
(SEQ ID NO: 25)
YQDED(pT)PPLEH(pS)PAHLP;
(SEQ ID NO: 26)
QDED(pT)PPLEHSPAHLP;
(SEQ ID NO: 27)
QDED(pT)PPLEH(pS)PAHLP;
(SEQ ID NO: 28)
KYRYQDED(pT)PPLEHSPAHL;
(SEQ ID NO: 29)
KYRYQDED(pT)PPLEH(pS)PAHL;
(SEQ ID NO: 30)
KYRYQDED(pT)PPLEHSPAH;
(SEQ ID NO: 31)
KYRYQDED(pT)PPLEH(pS)PAH;
(SEQ ID NO: 32)
KYRYQDED(pT)PPLEHSPA;
(SEQ ID NO: 33)
KYRYQDED(pT)PPLEH(pS)PA;
(SEQ ID NO: 34)
KYRYQDED(pT)PPLEH(pS)P;
(SEQ ID NO: 35)
KYRYQDED(pT)PPLEHSP;
(SEQ ID NO: 36)
KYRYQDED(pT)PPLEHS;
(SEQ ID NO: 37)
KYRYQDED(pT)PPLEH(pS);
(SEQ ID NO: 38)
KYRYQDED(pT)PPLEH;
or an antigenic portion thereof.

69. The immunoglobulin or the antigen binding fragment of claim 67, wherein the immunoglobulin or antigen binding fragment thereof comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 41, or 53, the VH-CDR2 comprises the amino acid sequence set forth in SEQ ID NO: 42, 54, or 62, the VH-CDR3 comprises the amino acid sequence set forth in SEQ ID NO: 43, 55, or 63, the VL-CDR1 comprises the amino acid sequence set forth in SEQ ID NO: 46, 58, or 66, the VL-CDR2 comprises the amino acid sequence set forth in SEQ ID NO: 47, and the VL-CDR3 comprises the amino acid sequence set forth in SEQ ID NO: 48, or 59.

70. The immunoglobulin or the antigen binding fragment of claim 67, wherein the immunoglobulin or antigen binding fragment thereof comprises a VH comprising the amino acid sequence set forth in SEQ ID NO: 40, 52, or 61, and a VL comprising the amino acid sequence set forth in SEQ ID NO: 45, 57, or 65.

71. The immunoglobulin or the antigen binding fragment of claim 67, which is a monoclonal antibody.

72. The immunoglobulin or the antigen binding fragment of claim 67, which is an antibody fragment that binds to PSD95:
a) phosphorylated at threonine 19;
b) phosphorylated at serine 25; or
c) phosphorylated at both threonine 19 and serine 25.

73. The immunoglobulin or the antigen binding fragment of claim 67, which binds to PSD95 phosphorylated at threonine 19 with a dissociation constant (Ka) value of at least 10⁻⁷ M.

74. A nucleic acid, comprising a nucleotide sequence encoding the immunoglobulin or the antigen binding fragment of claim 67.

75. A vector, comprising the nucleic acid of claim 74.

76. A host cell, comprising the expression vector of claim 75.

77. A method of diagnosis of a neurological disorder in a subject using the immunoglobulin or the antigen binding fragment of claim 67.

78. The method of claim 77, wherein the diagnosis is in vitro and/or in vivo.

79. A method for diagnosing a neurological disorder in a subject, comprising detecting in a sample from the subject threonine 19 phosphorylated PSD95, serine 25 phosphorylated PSD95, and/or both threonine 19 and serine 25 phosphorylated PSD95, wherein the sample is contacted with the immunoglobulin or the antigen binding fragment of claim 67.

80. The method of claim 79, wherein the diagnosis is in vitro and/or in vivo.

81-83. (canceled).

84. A method of diagnosing and treating a neurological disorder in a subject, the method comprising:
(a) obtaining a sample from the subject;
(b) contacting the sample from the subject with an immunoglobulin or an antigen binding fragment that specifically binds to PSD95 phosphorylated at threonine 19, serine 25, and/or PSD95 phosphorylated at both threonine 19 and serine 25;
(c) detecting the presence or absence of PSD95 phosphorylated at threonine 19, serine 25, and/or PSD95 phosphorylated at both threonine 19 and serine 25; or detecting the presence or absence of one or more complexes that include PSD95 phosphorylated at threonine 19, serine 25, and/or PSD95 phosphorylated at both threonine 19 and serine 25, and the immunoglobulin or the antigen binding fragment using a labeled secondary antibody;
(d) diagnosing the subject as having or as not having the neurological disorder based on the detection of the presence or absence of PSD95 phosphorylated at threonine 19, serine 25, and/or PSD95 phosphorylated at both threonine 19 and serine 25; or the one or more complexes; and
(e) administering an effective treatment to treat the subject having the neurological disorder.

85. The method of claim 84, further comprising detecting total PSD95 levels.

86. The method of claim 84, wherein the immunoglobulin or the antigen binding fragment are obtained by immunization with a polypeptide having an amino acid sequence selected from:
(SEQ ID NO: 1)
Ac-CDED(pT)PPLE;
(SEQ ID NO: 2)
CDED(pT)PPLE;
(SEQ ID NO: 3)
DED(pT)PPLE;
(SEQ ID NO: 4)
D(pT)PPLEHSP;
(SEQ ID NO: 5)
D(pT)PPLEH(pS)P;
(SEQ ID NO: 6)
D(pT)PPLEHSPA;
(SEQ ID NO: 7)
D(pT)PPLEH(pS)PA;
(SEQ ID NO: 8)
D(pT)PPLEHSPAH;
(SEQ ID NO: 9)
D(pT)PPLEH(pS)PAH;
(SEQ ID NO: 10)
ED(pT)PPLEHSPA;
(SEQ ID NO: 11)
ED(pT)PPLEH(pS)PA;
(SEQ ID NO: 12)
ED(pT)PPLEHSPAH;
(SEQ ID NO: 13)
ED(pT)PPLEH(pS)PAH;
(SEQ ID NO: 14)
DED(pT)PPLEHSPA;
(SEQ ID NO: 15)
DED(pT)PPLEH(pS)PA;
(SEQ ID NO: 16)
DED(pT)PPLEHSPAH;
(SEQ ID NO: 17)
DED(pT)PPLEH(pS)PAH;
(SEQ ID NO: 49)
Ac-CDED(pT)PPLEH(pS)PAH;
(SEQ ID NO: 50)
CDED(pT)PPLEH(pS)PAH;
(SEQ ID NO: 18)
KYRYQDED(pT)PPLEHSPAHLP;
(SEQ ID NO: 19)
KYRYQDED(pT)PPLEH(pS)PAHLP;
(SEQ ID NO: 20)
YRYQDED(pT)PPLEHSPAHLP;
(SEQ ID NO: 21)
YRYQDED(pT)PPLEH(pS)PAHLP;
(SEQ ID NO: 22)
RYQDED(pT)PPLEHSPAHLP;
(SEQ ID NO: 23)
RYQDED(pT)PPLEH(pS)PAHLP;
(SEQ ID NO: 24)
YQDED(pT)PPLEHSPAHLP;
(SEQ ID NO: 25)
YQDED(pT)PPLEH(pS)PAHLP;
(SEQ ID NO: 26)
QDED(pT)PPLEHSPAHLP;
(SEQ ID NO: 27)
QDED(pT)PPLEH(pS)PAHLP;
(SEQ ID NO: 28)
KYRYQDED(pT)PPLEHSPAHL;
(SEQ ID NO: 29)
KYRYQDED(pT)PPLEH(pS)PAHL;
(SEQ ID NO: 30)
KYRYQDED(pT)PPLEHSPAH;
(SEQ ID NO: 31)
KYRYQDED(pT)PPLEH(pS)PAH;
(SEQ ID NO: 32)
KYRYQDED(pT)PPLEHSPA;
(SEQ ID NO: 33)
KYRYQDED(pT)PPLEH(pS)PA;
(SEQ ID NO: 34)
KYRYQDED(pT)PPLEH(pS)P;
(SEQ ID NO: 35)
KYRYQDED(pT)PPLEHSP;
(SEQ ID NO: 36)
KYRYQDED(pT)PPLEHS;
(SEQ ID NO: 37)
KYRYQDED(pT)PPLEH(pS);
(SEQ ID NO: 38)
KYRYQDED(pT)PPLEH;
or an antigenic portion thereof.

87. The method of claim 84, wherein the immunoglobulin or the antigen binding fragment comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein the VH-CDR1 comprises the amino acid sequence set forth in SEQ ID NO: 41, or 53, the VH-CDR2 comprises the amino acid sequence set forth in SEQ ID NO: 42, 54, or 62, the VH-CDR3 comprises the amino acid sequence set forth in SEQ ID NO: 43, 55, or 63, the VL-CDR1 comprises the amino acid sequence set forth in SEQ ID NO: 46, 58, or 66, the VL-CDR2 comprises the amino acid sequence set forth in SEQ ID NO: 47, and the VL-CDR3 comprises the amino acid sequence set forth in SEQ ID NO: 48, or 59.

88. The method of claim 84, wherein the immunoglobulin or the antigen binding fragment comprises a VH comprising the amino acid sequence set forth in SEQ ID NO: 40, 52, or 61, and a VL comprising the amino acid sequence set forth in SEQ ID NO: 45, 57, or 65.

89-94. (canceled).

95. A kit for detecting PSD95 phosphorylated at threonine 19, serine 25, and/or PSD95 phosphorylated at both threonine 19 and serine 25 in a sample, the kit comprising: an immunoglobulin or an antigen binding fragment capable of binding specifically to PSD95 phosphorylated at threonine 19, serine 25, and/or PSD95 phosphorylated at both threonine 19 and serine 25 and a labeled immunoglobulin or a labeled antigen binding fragment immunoglobulin capable of binding specifically to the immunoglobulin or the antigen binding fragment capable of binding specifically to PSD95 phosphorylated at threonine 19, serine 25, and/or PSD95 phosphorylated at both threonine 19 and serine 25.

96-99. (canceled).


SEQUENCE LISTING (66 sequences)

SEQ ID NO: 1 - protein, 9 aa, synthetic construct
  SITE 5: note = phosphorylated threonine
  UNSURE 1: note = N terminal acylated
CDEDTPPLE

SEQ ID NO: 2 - protein, 9 aa, synthetic construct
  SITE 5: note = phosphorylated threonine
CDEDTPPLE

SEQ ID NO: 3 - protein, 8 aa, synthetic construct
  SITE 4: note = phosphorylated threonine
DEDTPPLE

SEQ ID NO: 4 - protein, 9 aa, synthetic construct
  SITE 2: note = phosphorylated threonine
DTPPLEHSP

SEQ ID NO: 5 - protein, 9 aa, synthetic construct
  SITE 2: note = phosphorylated threonine
  SITE 8: note = phosphorylated serine
DTPPLEHSP

SEQ ID NO: 6 - protein, 10 aa, synthetic construct
  SITE 2: note = phosphorylated threonine
DTPPLEHSPA

SEQ ID NO: 7 - protein, 10 aa, synthetic construct
  SITE 2: note = phosphorylated threonine
  SITE 8: note = phosphorylated serine
DTPPLEHSPA

SEQ ID NO: 8 - protein, 11 aa, synthetic construct
  SITE 2: note = phosphorylated threonine
DTPPLEHSPAH

SEQ ID NO: 9 - protein, 11 aa, synthetic construct
  SITE 2: note = phosphorylated threonine
  SITE 8: note = phosphorylated serine
DTPPLEHSPAH

SEQ ID NO: 10 - protein, 11 aa, synthetic construct
  SITE 3: note = phosphorylated threonine
EDTPPLEHSPA

SEQ ID NO: 11 - protein, 11 aa, synthetic construct
  SITE 3: note = phosphorylated threonine
  SITE 9: note = phosphorylated serine
EDTPPLEHSPA

SEQ ID NO: 12 - protein, 12 aa, synthetic construct
  SITE 3: note = phosphorylated threonine
EDTPPLEHSPAH

SEQ ID NO: 13 - protein, 12 aa, synthetic construct
  SITE 3: note = phosphorylated threonine
  SITE 9: note = phosphorylated serine
EDTPPLEHSPAH

SEQ ID NO: 14 - protein, 12 aa, synthetic construct
  SITE 4: note = phosphorylated threonine
DEDTPPLEHSPA

SEQ ID NO: 15 - protein, 12 aa, synthetic construct
  SITE 4: note = phosphorylated threonine
  SITE 10: note = phosphorylated serine
DEDTPPLEHSPA

SEQ ID NO: 16 - protein, 13 aa, synthetic construct
  SITE 4: note = phosphorylated threonine
DEDTPPLEHSPAH

SEQ ID NO: 17 - protein, 13 aa, synthetic construct
  SITE 4: note = phosphorylated threonine
  SITE 10: note = phosphorylated serine
DEDTPPLEHSPAH

SEQ ID NO: 18 - protein, 20 aa, synthetic construct
  SITE 9: note = phosphorylated threonine
KYRYQDEDTPPLEHSPAHLP

SEQ ID NO: 19 - protein, 20 aa, synthetic construct
  SITE 9: note = phosphorylated threonine
  SITE 15: note = phosphorylated serine
KYRYQDEDTPPLEHSPAHLP

SEQ ID NO: 20 - protein, 19 aa, synthetic construct
  SITE 8: note = phosphorylated threonine
YRYQDEDTPPLEHSPAHLP

SEQ ID NO: 21 - protein, 19 aa, synthetic construct
  SITE 8: note = phosphorylated threonine
  SITE 14: note = phosphorylated serine
YRYQDEDTPPLEHSPAHLP

SEQ ID NO: 22 - protein, 18 aa, synthetic construct
  SITE 7: note = phosphorylated threonine
RYQDEDTPPLEHSPAHLP

SEQ ID NO: 23 - protein, 18 aa, synthetic construct
  SITE 7: note = phosphorylated threonine
  SITE 13: note = phosphorylated serine
RYQDEDTPPLEHSPAHLP

SEQ ID NO: 24 - protein, 17 aa, synthetic construct
  SITE 6: note = phosphorylated threonine
YQDEDTPPLEHSPAHLP

SEQ ID NO: 25 - protein, 17 aa, synthetic construct
  SITE 7: note = phosphorylated threonine
  SITE 12: note = phosphorylated serine
YQDEDTPPLEHSPAHLP

SEQ ID NO: 26 - protein, 16 aa, synthetic construct
  SITE 5: note = phosphorylated threonine
QDEDTPPLEHSPAHLP

SEQ ID NO: 27 - protein, 16 aa, synthetic construct
  SITE 5: note = phosphorylated threonine
  SITE 11: note = phosphorylated serine
QDEDTPPLEHSPAHLP

SEQ ID NO: 28 - protein, 19 aa, synthetic construct
  SITE 9: note = phosphorylated threonine
KYRYQDEDTPPLEHSPAHL

SEQ ID NO: 29 - protein, 19 aa, synthetic construct
  SITE 6: note = phosphorylated threonine
  SITE 15: note = phosphorylated serine
KYRYQDEDTPPLEHSPAHL

SEQ ID NO: 30 - protein, 18 aa, synthetic construct
  SITE 9: note = phosphorylated threonine
KYRYQDEDTPPLEHSPAH

SEQ ID NO: 31 - protein, 18 aa, synthetic construct
  SITE 9: note = phosphorylated threonine
  SITE 15: note = phosphorylated serine
KYRYQDEDTPPLEHSPAH

SEQ ID NO: 32 - protein, 17 aa, synthetic construct
  SITE 9: note = phosphorylated threonine
KYRYQDEDTPPLEHSPA

SEQ ID NO: 33 - protein, 17 aa, unidentified
  SITE 9: note = phosphorylated threonine
  SITE 15: note = phosphorylated serine
KYRYQDEDTPPLEHSPA

SEQ ID NO: 34 - protein, 16 aa, synthetic construct
  SITE 9: note = phosphorylated threonine
  SITE 15: note = phosphorylated serine
KYRYQDEDTPPLEHSP

SEQ ID NO: 35 - protein, 16 aa, synthetic construct
  SITE 9: note = phosphorylated threonine
KYRYQDEDTPPLEHSP

SEQ ID NO: 36 - protein, 15 aa, synthetic construct
  SITE 9: note = phosphorylated threonine
KYRYQDEDTPPLEHS

SEQ ID NO: 37 - protein, 15 aa, synthetic construct
  SITE 9: note = phosphorylated threonine
  SITE 15: note = phosphorylated serine
KYRYQDEDTPPLEHS

SEQ ID NO: 38 - protein, 14 aa, synthetic construct
  SITE 9: note = phosphorylated threonine
KYRYQDEDTPPLEH

SEQ ID NO: 39 - DNA, 366 bp, synthetic construct
  source 1..366: mol_type = other DNA
gaggtgcggcttgttgagtctggtggaggattggtgcagcctaaagggtcattgaaaatctcatgtgcagcctctggattcaccttcaacacctacgccatgaactgggtccgccaggctccaggaaagggtttggaatggattgctcgcataagaagtaaaagtaataattatgcaacatattatgacgattcagtgaaagacaggttcaccatctccagagatgattcacaaagcatgctctatctgcaaatgaacaacttgaaaactgaggacacagccatgtattactgtgtgagacggggggatggtcactacatctggtttgcttactggggccaagggactctggtcactgtctctgca

SEQ ID NO: 40 - protein, 122 aa, synthetic construct
EVRLVESGGGLVQPKGSLKISCAASGFTFNTYAMNWVRQAPGKGLEWIARIRSKSNNYATYYDDSVKDRFTISRDDSQSMLYLQMNNLKTEDTAMYYCVRRGDGHYIWFAYWGQGTLVTVSA

SEQ ID NO: 41 - protein, 8 aa, synthetic construct
GFTFNTYA

SEQ ID NO: 42 - protein, 10 aa, synthetic construct
IRSKSNNYAT

SEQ ID NO: 43 - protein, 13 aa, synthetic construct
VRRGDGHYIWFAY

SEQ ID NO: 44 - DNA, 336 bp, synthetic construct
  source 1..336: mol_type = other DNA
gatgttttgatgacccaaactccactctccctgcctgtcagtcttggagatcaagcctccatctcttgcagatctagtcagagcattgtaaatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 45 - protein, 112 aa, synthetic construct
DVLMTQTPLSLPVSLGDQASISCRSSQSIVNSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 46 - protein, 11 aa, synthetic construct
QSIVNSNGNTY

SEQ ID NO: 47 - skipped

SEQ ID NO: 48 - protein, 9 aa, synthetic construct
FQGSHVPLT

SEQ ID NO: 49 - protein, 14 aa, synthetic construct
  SITE 5: note = phosphorylated threonine
  SITE 11: note = phosphorylated serine
  UNSURE 1: note = N terminal acylated
CDEDTPPLEHSPAH

SEQ ID NO: 50 - protein, 14 aa, synthetic construct
  SITE 5: note = phosphorylated threonine
  SITE 11: note = phosphorylated serine
CDEDTPPLEHSPAH

SEQ ID NO: 51 - DNA, 367 bp, synthetic construct
  source 1..367: mol_type = other DNA
gaggtgcagcttgttgagactggtggaggattggtgcagcctaaagggtcattgaaactctcatgtgcagcctctggattcaccttcaataccaatgccatgaattgggtccgccaggctccaggaaagggtttggaatggattgctcgcataagaactaaaagtaataattatggaacatattatgccgattcagtgaaagacaggttcaccatttccagagatgattcacaaaacatactttatctgcaaatgaacaacttgaaaactgaggacacagccatgtattactgtgtgagagggaagatcaactatgattacgggtttggttactggggccaagggactctggtcactgtctctgcag

SEQ ID NO: 52 - protein, 122 aa, synthetic construct
EVQLVETGGGLVQPKGSLKLSCAASGFTFNTNAMNWVRQAPGKGLEWIARIRTKSNNYGTYYADSVKDRFTISRDDSQNILYLQMNNLKTEDTAMYYCVRGKINYDYGFGYWGQGTLVTVSA

SEQ ID NO: 53 - protein, 8 aa, synthetic construct
GFTFNTNA

SEQ ID NO: 54 - protein, 10 aa, synthetic construct
IRTKSNNYGT

SEQ ID NO: 55 - protein, 13 aa, synthetic construct
VRGKINYDYGFGY

SEQ ID NO: 56 - DNA, 337 bp, synthetic construct
  source 1..337: mol_type = other DNA
gatgttttgatgacccaaactccactctccctgcctgtcagtcttggagatcaagcctccatctcttgcagatctagtcagagcattgtacatagtaatggaaacacctatttagaatggttcctgcagaaaccaggccagtctccaaagctcctgatctacaaagtttccaaccgattttctggggtcccagacaggttcagtggcagtggatcagggactgatttcacactcaagatcagcagaatggaggctgaggatctgggagtttattattgttttcaaggttcacatgttccttacacgttcggaggggggaccaagctggaaataaaac

SEQ ID NO: 57 - protein, 112 aa, synthetic construct
DVLMTQTPLSLPVSLGDQASISCRSSQSIVHSNGNTYLEWFLQKPGQSPKLLIYKVSNRFSGVPDRFSGSGSGTDFTLKISRMEAEDLGVYYCFQGSHVPYTFGGGTKLEIK

SEQ ID NO: 58 - protein, 11 aa, synthetic construct
QSIVHSNGNTY

SEQ ID NO: 59 - protein, 9 aa, synthetic construct
FQGSHVPYT

SEQ ID NO: 60 - DNA, 367 bp, synthetic construct
  source 1..367: mol_type = other DNA
gaggtgcagcttgttgagactggtggaggattggtgcagcctaaagggtcattgaaactctcatgtgcagcctctggattcaccttcaataccaatgccatgaattgggtccgccaggctccaggaaagggtttggaatggattgctcgcataagaactaaaagtaatcattatggaacatattatgccgattcagtgaaagacaggttcaccatttccagagatgattcacaaaacatactttatctgcaaatgaacaacttgaaaactgaggacacagccatgtattactgtgtgagaggccagatcaactatgattacgggtttggttactggggccaagggactctggtcactgtctctgcag

SEQ ID NO: 61 - protein, 122 aa, synthetic construct
EVQLVETGGGLVQPKGSLKLSCAASGFTFNTNAMNWVRQAPGKGLEWIARIRTKSNHYGTYYADSVKDRFTISRDDSQNILYLQMNNLKTEDTAMYYCVRGQINYDYGFGYWGQGTLVTVSA

SEQ ID NO: 62 - protein, 10 aa, synthetic construct
IRTKSNHYGT

SEQ ID NO: 63 - protein, 13 aa, synthetic construct
VRGQINYDYGFGY

SEQ ID NO: 64 - DNA, 337 bp, synthetic construct
  source 1..337: mol_type = other DNA
gatgttttgatgacccaaactccactctccctgcctgtcagtcttggagatcaagcctccatctcttgcagatctagtcagaacattgtacatagtaatggaaacacctatttagaatggtacctgcagaaaccaggccagtctccaaagctcctgatctacaaagtttccaaccgattttctggggtcccagacaggttcagtggcagtggatcagggactgatttcacactcaagatcagcagaatggaggctgaggatctgggagtttattattgttttcaaggttcacatgttccttacacgttcggaggggggaccaagctggaaataaaac

SEQ ID NO: 65 - protein, 112 aa, synthetic construct
DVLMTQTPLSLPVSLGDQASISCRSSQNIVHSNGNTYLEWYLQKPGQSPKLLIYKVSNRFSGVPDRFSGSGSGTDFTLKISRMEAEDLGVYYCFQGSHVPYTFGGGTKLEIK

SEQ ID NO: 66 - protein, 11 aa, synthetic construct
QNIVHSNGNTY